(19) United States
(12) Patent Application Publication
(10) Pub. No.: US 2026/0043035 A1
(43) Pub. Date: Feb. 12, 2026

(54) NATURE-DERIVED LIPID PARTICLES FOR USE IN AGRICULTURE

(71) Applicant: INVAIO SCIENCES, INC., Cambridge, MA (US)

(72) Inventors: Ana Isabel CAÑO-DELGADO, Cambridge, MA (US); Hailey Deanne HANSCOM, Cambridge, MA (US); Jeffery Allen HUGHES, Cambridge, MA (US); Maria del Mar MARQUÉS-BUENO, Cambridge, MA (US); Maria MERINO-GÓMEZ, Cambridge, MA (US); Andrés RICO-MEDINA, Cambridge, MA (US); Marta RIERA-BONET, Cambridge, MA (US); Robbie Charles SABATELLE, Cambridge, MA (US)

(21) Appl. No.: 19/294,529

(22) Filed: Aug. 8, 2025

Related U.S. Application Data
(60) Provisional application No. 63/681,050, filed on Aug. 8, 2024.

(51) Int. Cl. C12N 15/82; A01N 25/22; A01N 25/26; A01P 21/00; C12N 9/22; C12N 15/10; C12N 15/113; C12N 15/88

(52) U.S. Cl. CPC C12N 15/82; A01N 25/22; A01N 25/26; A01P 21/00; C12N 9/222; C12N 15/10; C12N 15/113; C12N 15/88; C12N 2310/20; C12N 2510/00

(57) ABSTRACT

Compositions and methods are provided for delivering hydrophilic functional agents to plants using lipid nanoparticles (NLPs). The NLPs comprise a hydrophilic core surrounded by a lipid shell including at least one sterol, one phospholipid, and one cationic or ionizable lipid. The hydrophilic core enables encapsulation and stabilization of water-soluble agents such as nucleic acids, peptides, small molecules, or plant hormones. The NLPs enhance uptake, protect cargo from degradation, and facilitate delivery to internal plant tissues, including meristems and vascular structures. Methods of treating plants and plant parts with the compositions are also provided.

### CROSS REFERENCE TO RELATED APPLICATIONS

This application claims priority to provisional application U.S. Ser. No. 63/681,050, filed Aug. 8, 2024, which is hereby incorporated herein by reference in its entirety.

### SEQUENCE LISTING XML

The instant application contains a sequence listing, which has been submitted in XML file format by electronic submission and is hereby incorporated by reference in its entirety. The XML file, created on Aug. 6, 2025, is named P15122US01.xml and is 2,603 bytes in size.

### BACKGROUND

The effective delivery of heterologous functional agents (e.g., a polynucleotide) to a plant can be limited by the degree to which the agent can penetrate plant cell barriers. In particular, the barrier formed by the plant cell wall poses a challenge to cellular uptake of agents useful in agriculture. Therefore, there is a need in the art for methods and compositions promoting the cellular uptake of functional agents by plants.

### SUMMARY

Described herein are nature-derived lipid particles (NLPs) useful for the delivery of one or more hydrophilic functional agents to a plant cell. In some embodiments, the hydrophilic functional agent is a polynucleotide. In some embodiments, the hydrophilic functional agent is a polynucleotide.

In some embodiments, the hydrophilic functional agent is RNA. In some embodiments, the hydrophilic functional agent is DNA. In some embodiments, the hydrophilic functional agent is a peptide.

In some embodiments, the NLPs described herein comprise a hydrophilic core. In such embodiments, the NLP is configured to encapsulate one or more hydrophylic functional agents within the lipid bilayer or hydrophylic interior. In some aspects, the hydrophilic core is surrounded by a monolayer or multilamellar phospholipid shell.

In some aspects, herein is provided a composition for delivering a heterologous functional agent to a plant cell, the composition comprising a plurality of NLPs, wherein each NLP comprises: at least one sterol; at least one quaternary ammonium salt lipid and/or at least one tertiary amine lipid; at least one phospholipid; and at least one heterologous functional agent; wherein the NLP comprises a hydrophilic core, and wherein the heterologous functional agent is encapsulated in the NLP. In some embodiments, the NLP is an RNA-NLP (rNLP) encapsulating one or more RNA functional agents. In some embodiments, the NLP is a DNA-NLP (dNLP) encapsulating one or more DNA functional agents. In some embodiments, the NLP is a peptide-NLP (pNLP) encapsulating one or more peptide functional agents.

In some embodiments, the NLP comprises one or more sterols. Not wishing to be bound by a particular theory, the inclusion of one or more sterols in an NLP formulation contributes to the structural stability and functional performance of the nanoparticle. In some embodiments, the one or more sterols are included in the NLP at a molar ratio of 5% to 50%, such as from 10-40%, or preferably 20-35%, based on the total molar lipid composition. In some embodiments the sterol is cholesterol or sitosterol. In some embodiments, the sterol is a phytosterol. In some embodiments, the NLP comprises one of more phytosterols selected from the group consisting of β-sitosterol, stigmasterol, campesterol, fucosterol, and brassicasterol. In certain embodiments, β-sitosterol is used as a complete substitute for cholesterol, while in other embodiments, phytosterols are used in combination with cholesterol.

In some embodiments the at least one quaternary ammonium salt lipid is selected from the group consisting of SM-102 N-oxide, ALC-0315 N-oxide, Hexadecanedioic Acid Mono-L-carnitine Ester Chloride, Octadecanedioic Acid Mono-L-carnitine Ester Chloride, 14:0 TAP, 16:0 TAP, 18:0 TAP, DGTS (1,2-dipalmitoyl-sn-glycero-3-O-4′-[N,N,N-trimethyl]-homoserine), DC-6-14, 12:0 EPC, 14:0 EPC, 16:0 EPC, 18:0 EPC, 16:0-18:1 EPC, 1-Palmitoyl-2-arachidoyllecithin, iPhos-lipid1, 18:0 DDAB (DDAB), DOTAP, DOTMA, DODAC, DORI, DOBAQ, MVL5 and DOSPA. In some embodiments the at least one quaternary ammonium salt lipid is DOTAP or DDAB.

In some embodiments the at least one tertiary amine lipid is selected from the group consisting of 113-O12B, 113-O16B, 14:0 DAP, 16:0 DAP, 1O14, 246C10, 304O13, 306-N16B, 306-O12B, 306-O12B-3, 306Oi10, 306Oi9-cis2, 4A3-SC8503O13, 80-O16B, 93-O17O, 93-O17S, 98N12-5, 9A1P9, 98N12-5, A12-Iso5-2DC18, A18-Iso5-2DC18, AA3-DLin, AA-T3A-C12, Al-28, ALC-0315 (ALC-315), ATX-001, ATX-100, BAMEA-O16B, C12-113, C12-200, C12-SPM, C13-112-tetra-tail, C13-112-tri-tail, C13-113-tetra-tail, C13-113-tri-tail, C14-4, C14-SPM, C3-K2-E14, cKK-E12, cKK-E12, cKK-E15, CL1, DLin-DMA, DLin-KC2-DMA (KC2), D-Lin-MC2-DMA, D-Lin-MC3-DMA (MC3), D-Lin-MC4-DMA, DODAP (18:1), DODMA, DOG-IM4, FITS, G0-C14, GL67, IAJD249, IAJD93, IC8, iPhos-lipid2, iPhos-lipid3, iPhos-lipid4, L13, L14, L15, L16, L2, L3, L319, L9, Lipid 10, Lipid 14, Lipid 16, Lipid 2, Lipid 23, Lipid 29, Lipid 5, Lipid 8, Lipid A4, Lipid A6, Lipid A9, Lipid AX4, Lipid C24, Lipid Catechol, Lipid III-45, Lipid R6, LP01, MVL5, NT1-O14B, OC2-K3-E10, OF-02, OF-C4-Deg-Lin, OF-Deg-Lin, PPZ-A10, RCB-4-8, RM 133-3, RM 137-15, SM-102, SSPalmM, SSPalmO-Phe, TCL053, TT3, YK-009, YSK05, and ZA3-Ep10. In some embodiments the at least one tertiary amine lipid is selected from the group consisting of MC3, KC2, DODMA, SM-102 and ALC-315.

In some embodiments the at least one phospholipid is DOPE, DSPC, soybean lecithin, or sunflower lecithin. In some embodiments the soybean lecithin is de-oiled soybean lecithin. In some embodiments the sunflower lecithin is de-oiled sunflower lecithin.

In some embodiments the composition further comprises a surface modifier. In some embodiments the surface modifier is a pegylated lipid. In some embodiments the pegylated lipid is selected from the group consisting of PEG5K PE 14:0, PEG2K PE 14:0, and PEG2K PE 18:0. In some embodiments the pegylated lipid is present at a molar percentage of about 0.1-10%.

In some embodiments the composition further comprises a pectin derivative. In some embodiments the pectin derivative is a hydrophobically modified pectin derivative. In some embodiments the pectin derivative is present at a molar percentage of about 0.1-10%.

In some embodiments the composition further comprises a boron acid lipid. In some embodiments the boron acid lipid is present at a molar percentage of about 0.1-10%.

In some embodiments the composition comprises 25-45 mol % of at least one sterol, 15-60 mol % of at least one synthetic lipid, and 5-55 mol % of at least one phospholipid. In some embodiments the composition comprises about 35 mol % of at least one sterol, about 25 mol % of at least one synthetic lipid, and about 35 mol % of at least one phospholipid.

In some embodiments the heterologous functional agent is a polynucleotide. In some embodiments the polynucleotide is chosen from an mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), an ADAR targeting oligonucleotide, an antisense oligonucleotide, a long non-coding RNA, a ceDNA, a minicircle, a miniplasmid, a viroid, a virus, or a DNA molecule encoding any of these RNAs. In some embodiments the polynucleotide is an mRNA, a siRNA, or a gRNA. In some embodiments the polynucleotide is a plasmid.

In some embodiments, the heterologous functional agent is a hydrophilic small molecule, such as: Phytohormones: salicylic acid, abscisic acid (ABA), gibberellic acid, cytokinins, brassinosteroids; Herbicides: glufosinate, glyphosate salts, bentazon; Antibiotics: streptomycin, oxytetracycline, kasugamycin; Antifungals: fosetyl-Al, phosphorous acid, thiophanate-methyl; Elicitors: chitosan, harpins, oligogalacturonides; Chelators and micronutrients: EDTA, ZnSO₄, Fe-EDDHA; Amino acids and osmolytes: proline, glycine betaine; Biostimulants: humic substances, seaweed extracts, polyamines. These agents may be encapsulated in NLPs either alone or in combination with nucleic acids or peptides, to modify plant physiology or provide protection against pests and stress.

In some embodiments, the NLP composition comprises a combination of two or more hydrophilic functional agents, such as a nucleic acid and a small molecule elicitor, or a peptide and an antibiotic. These combinations may target multiple biological pathways to enhance efficacy.

Physiochemical characteristics of the NLPs described herein may influence uptake and distribution in a plant. In some embodiments, the size of the NLPs ranges between 50 and 500 nm. In some embodiments the size of the NLP is below 200 nm. In some embodiments, the zeta potential of the NLPs ranges between 0 and +50 mV. In some embodiments the zeta potential of the NLPs ranges between 0 and −50 mV. In some embodiments, the NLPs described herein exhibit a polydispersity index (PDI) that reflects the uniformity of particle size distribution in the formulation. In certain embodiments, the NLPs exhibit a PDI of less than 0.3, such as less than 0.25, less than 0.2, or less than 0.15. In some preferred embodiments, the NLPs exhibit a PDI in the range of 0.05 to 0.2, which reflects a narrow and well-controlled size distribution. In some embodiments, a PDI of 0.1 to 0.2 is sufficient to achieve systemic delivery and distribution throughout multiple plant tissues, including movement from root to shoot. In certain embodiments, formulations exhibiting both particle diameters of less than 200 nm and PDI values less than 0.2 demonstrate enhanced systemicity and biological efficacy.

In some embodiments the NLPs comprise at least one a phospholipid bilayer. In some embodiments the NLPs have a micellar structure.

In some embodiments the composition further comprises an unencapsulated heterologous functional agent. In some embodiments the unencapsulated heterologous functional agent is selected from the group consisting of a pesticidal agent, a fertilizing agent, a herbicidal agent, a plant-modifying agent, an insect attractant, a plant growth promoting agent, a biostimulant, and a plant immunity elicitor.

In some aspects, herein is provided an NLP composition for delivering a polynucleotide to a plant cell, the composition comprising a molar percentage of about 35 mol % cholesterol, about 22 mol % DDAB, about 25 mol % DOPE, and about 18 mol % sunflower lecithin, and a polynucleotide. In some embodiments the polynucleotide is an mRNA, an siRNA, a microRNA, an antisense RNA or a gRNA.

In some aspects, herein is provided a composition for delivering at least one heterologous functional agent to a plant cell, the composition comprising a first and a second plurality of NLPs, wherein each plurality of NLPs has the composition of any of the preceding embodiments, and wherein the first and the second plurality of NLPS each comprise different heterologous functional agents.

In some aspects, herein is provided a composition for use in delivering a heterologous functional agent to a plant cell, the composition comprising a plurality of NLPs, wherein each NLP comprises: at least one sterol; at least one quaternary ammonium salt lipid and/or at least one tertiary amine lipid; at least one phospholipid; and a heterologous functional agent, wherein the NLP comprises a hydrophilic core, and wherein the heterologous functional agent is encapsulated in the NLP.

In some aspects, herein is provided a composition comprising a plurality of NLPs, wherein the NLPs are produced by a process comprising the steps of: (a) providing an organic phase comprising: at least one sterol; at least one quaternary ammonium salt lipid and/or at least one tertiary amine lipid; at least one phospholipid; (b) providing an aqueous phase comprising at least one heterologous functional agent; (c) mixing the organic phase and the aqueous phase; thereby forming the NLPs.

In some aspects, herein is provided a method for delivering a heterologous functional agent to a plant cell, the method comprising: contacting the plant cell with the NLP composition of any of the preceding embodiments; thereby delivering the heterologous functional agent to the plant cell.

In some aspects, herein is provided a method for increasing the uptake of a heterologous functional agent by a plant cell, the method comprising: (a) encapsulating a heterologous functional agent in an NLP comprising: at least one sterol; at least one quaternary ammonium salt lipid and/or at least one tertiary amine lipid; at least one phospholipid; and (b) contacting the plant cell with the NLP composition comprising the heterologous functional agent; thereby increasing the uptake of the heterologous functional agent as compared to a heterologous agent not comprised in an NLP composition.

In some aspects, herein is provided a method for increasing the fitness of a plant, the method comprising: delivering to the plant an effective amount of the composition of any of the preceding embodiments, wherein the method increases the fitness of the plant relative to an untreated plant.

In some aspects, herein is provided a method for decreasing the fitness of a plant, the method comprising: delivering to the plant an effective amount of the composition of any of the preceding embodiments, wherein the method decreases the fitness of the plant relative to an untreated plant.

In some aspects, herein is provided a method for modifying a nucleic acid in a plant cell, the method comprising: contacting the plant cell with the composition of any of the preceding embodiments. In some embodiments, the plant comprises an expression cassette for a CRISPR endonuclease.

### BRIEF DESCRIPTION OF THE DRAWINGS

FIG. 1A-1F depict rNLP uptake in Arabidopsis roots. FIG. 1A-1C, roots treated with FORM120; FIG. 1D-1F, roots treated with FORM030. FIG. 1A, the epidermal cell layer in the root meristem of Arabidopsis. Marked with arrows, circular shapes of increased fluorescence are cell nuclei expressing GFP-NLS after rNLP FORM0120 uptake; FIG. 1B, three-dimensional reconstruction from Z-stack in mature root epidermal cell layer. Arrow points to a nuclei expressing GFP-NLS. Image was taken after 24h of incubation with FORM126; FIG. 1C: different angle of the same cell as presented in FIG. 1B. Marked with an arrow, the nuclei in the inner of the cell. FIG. 1D-1F: roots treated with FORM030 not comprising RNA, showing no nuclear localization.

FIG. 2A-2F depict rNLP uptake in Arabidopsis leaves. FIG. 2A-2D, cells treated with FORM138; FIG. 2E-2F, cells treated with FORM138-1 “neg” (comprising no RNA). FIG. 2A, leaf pavement cell expressing GFP-NLS in the nuclei, marked with an arrow. Image taken of leaves upon treatment with FORM138 after 6 hours of incubation; FIG. 2B, three-dimensional reconstruction of the leaf shown in FIG. 2A, from a z-stack; FIG. 2C, leaf pavement cell expressing GFP-NLS in the nuclei, marked with an arrow. Image taken of leaves upon treatment with FORM138 after 6 hours of incubation; FIG. 2D, three-dimensional reconstruction of the image in FIG. 2C from a z-stack. Representative images are shown of 18 repeats.

FIG. 3A-3G depict uptake of rNLPs comprising rNLP-GFP by corn roots. FIG. 3A, epidermis corn root cells expressing the NLS-GFP mRNA. Expression was assessed 16 hours after rNLP treatment. FIG. 3A, negative control (MS buffer); FIG. 3B, NLP formulation without RNA (FORM030); FIG. 3C, FORM078; FIG. 3D, FORM079; FIG. 3E, FORM126; FIG. 3F, FORM127; FIG. 3G, FORM138. Arrows point to nuclei expressing GFP.

FIG. 4A-4K depict GFP mRNA expression in Nicotiana benthamiana leaves treated with different rNLPs. rNLPs were infiltrated in the leaves using 1 ml needleless syringe and visualized on a confocal microscope after 5 to 6 days. FIG. 4A, FORM078; FIG. 4B, FORM079; FIG. 4C, FORM065; FIG. 4D, FORM074; FIG. 4E, FORM075; FIG. 4F, FORM076; FIG. 4G, FORM085; FIG. 4H, FORM084; FIG. 4I, FORM029; GFP is localized close to the plasma membrane FIG. 4J: non infiltrated negative control; FIG. 4K: NLP FORM066 without GFP RNA. Note that some fluorescence is observed in the latter, due to chloroplast autofluorescence.

FIG. 5A-5H depict GFP silencing in Nicotiana benthamiana leaves of 16C GFP-ox plants. rNLPs formulations comprising siRNA were abrased in the leaves, and leaves were visualized under UV light after 5 to 6 days. Spots with reduced fluorescence were observed (indicated with arrows) correspond to the silencing phenotype. FIG. 5A-5B, FORM135 containing siRNA-GFP; FIG. 5C-5D, FORM136 containing siRNA-GFP; FIG. 5E-5F, Leaves abrased with unencapsulated siRNA (1 ug/ul); FIG. 5G, non-treated 16C control leaf; and FIG. 5H, non-treated WT control leaf.

FIG. 6 depicts the visualization of NLP-RNA delivery in Arabidopsis and Maize leaves. (a-c) 12-day old Arabidopsis seedlings were incubated NLP containing the RNA of the mTurquoise fused with the nuclear exportation signal (NLS) for 24 hours. a) Formulation without RNA. b) formulation #78 containing the mRNA of the mTurquoise. c) formulation #138 containing the mRNA of the mTurquoise. (d-f) sections of 3-week old maize leaves incubated NLP containing the RNA of the mTurquoise fused with the nuclear exportation signal (NLS) for 24 hours. d) Formulation without RNA. e) formulation #78 containing the mRNA of the mTurquoise. f) formulation #138 containing the mRNA of the mTurquoise. Arrows point nuclei expressing mTurquoise.

FIG. 7 shows sections of 3-week old maize leaves incubated in NLP formulations containing the RNA of the mTurquoise fused with the nuclear exportation signal (NLS) for 24 hours. The upper left panel shows a micrograph of a corn leaf treated with an empty Formulation 377 (without RNA). FORM78, FORM138, FORM209, FORM377, FORM378 contain the mRNA of the mTurquoise. Arrows point nuclei expressing mTurquoise.

FIG. 8 shows photographs of gel electrophoresis detecting PCR amplicons of pBR322 from Root (R), Mesocotyl (M) or Shoot (S) tissue isolated from corn seedlings treated with Naked DNA (no NLP formulation), dNLP Form377 (F377), dNLP Form380 (F380), dNLP Form381 (F381) or negative control (empty (no cargo) NLP or MS media). Detection of a band between 1000 and 500 bp indicates the presence of pBR322 cargo in the tissue. Legend: R) Root, S) Shoot, NT) No transport, T) Transport.

### DETAILED DESCRIPTION

Featured herein are NLPs comprising a hydrophilic core for use in agriculture. NLPs as described herein may be used to promote the uptake of a hydrophilic functional agent in a plant cell.

NLPs as described herein may used in a variety of agricultural applications, e.g. to accomplish improved yield of a plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional agricultural agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. An increase in the fitness of plant can also be measured by other means, such as an increase or improvement of the vigor rating, increase in the stand (the number of plants per unit of area), increase in plant height, increase in stalk circumference, increase in plant canopy, improvement in appearance (such as greener leaf color as measured visually), improvement in root rating, increase in seedling emergence, protein content, increase in leaf size, increase in leaf number, fewer dead basal leaves, increase in tiller strength, decrease in nutrient or fertilizer requirements, increase in seed germination, increase in tiller productivity, increase in flowering, increase in seed or grain maturation or seed maturity, fewer plant verse (lodging), increased shoot growth, or any combination of these factors, by a measurable or noticeable amount over the same factor of the plant produced under the same conditions, but without the administration of the instant compositions or with application of conventional agricultural agents.

### Definitions

As used herein, the term “heterologous” refers to an agent that is either (1) exogenous to the plant (e.g., originating from a source that is not the plant from which the NLP is produced) or (2) endogenous to the plant from which the NLP is produced, but is present in the NLP (e.g., using loading, genetic engineering, in vitro or in vivo approaches) at a concentration that is higher than that found in nature (e.g., as found in a naturally-occurring plant extracellular vesicle).

As used herein, an “agriculturally acceptable” carrier is one that is suitable for use in agriculture, e.g., for use on plants. In certain embodiments, the agriculturally acceptable carrier does not have undue adverse side effects to the plants, the environment, or to humans or animals who consume the resulting agricultural products derived therefrom commensurate with a reasonable benefit/risk ratio.

As used herein, “delivering” or “contacting” refers to applying an NLP composition as described herein either directly to a plant, animal (e.g., insect, nematode, etc.), fungus, or bacterium, or adjacent to the plant, animal, fungus, or bacterium, in a region where the composition is effective to alter the fitness of the plant, animal, fungus, or bacterium. In methods where the composition is directly contacted with a plant, animal, fungus, or bacterium, the composition may be contacted with the entire plant, animal, fungus, or bacterium or with only a portion of the plant, animal, fungus, or bacterium. In some embodiments, the NLP composition may translocate across the plant cell barrier. In some embodiments, the NLPs are ingested by a plant pest, such as an insect or nematode.

As used herein, “decreasing the fitness of a plant” refers to any disruption of the physiology of a plant (e.g., a weed) as a consequence of administration of a composition described herein (e.g., an NLP composition including a heterologous functional agent as described herein), including, but not limited to, decreasing a population of a plant (e.g., a weed) by about 10%, 20%, 30%, 40%, 50%, 60%, 70%, 80%, 90%, 95%, 99%, 100% or more. A decrease in plant fitness can be determined in comparison to a plant to which the composition has not been administered.

As used herein, the term “effective amount,” “effective concentration,” or “concentration effective to” refers to an amount of a heterologous functional agent provided in a NLP composition as described herein, sufficient to affect the recited result or to reach a target level (e.g., a predetermined or threshold level) in or on a target organism.

As used herein, “increasing the fitness of a plant” refers to an increase in the production of the plant, for example, an improved yield, improved vigor of the plant, or improved quality of the harvested product from the plant as a consequence of administration of a composition described herein (e.g., an NLP composition including a heterologous functional agent as described herein). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional agricultural agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of raw material used. An increase in the fitness of plant can also be measured by other means, such as an increase or improvement of the vigor rating, increase in the stand (the number of plants per unit of area), increase in plant height, increase in stalk circumference, increase in plant canopy, improvement in appearance (such as greener leaf color as measured visually), improvement in root rating, increase in seedling emergence, protein content, increase in leaf size, increase in leaf number, fewer dead basal leaves, increase in tiller strength, decrease in nutrient or fertilizer requirements, increase in seed germination, increase in tiller productivity, increase in flowering, increase in seed or grain maturation or seed maturity, fewer plant verse (lodging), increased shoot growth, or any combination of these factors, by a measurable or noticeable amount over the same factor of the plant produced under the same conditions, but without the administration of the instant compositions or with application of conventional agricultural agents.

As used herein, the term “heterologous” refers to an agent that is exogenous to the plant or plant part that is contacted with the NLP (e.g., originating from a source that is not the plant itself, e.g., the pesticidal agent incorporated in an NLP may be heterologous). In some embodiments, one or more components of the NLP are heterologous.

As used herein, the term “functional agent” refers to any molecule or composition that confers a desired biological effect when delivered to a plant, plant part, plant pest or associated organism. Functional agents include but are not limited to: Polynucleotides (e.g., RNA, DNA, siRNA, miRNA, antisense RNA, CRISPR components), Polypeptides (e.g., enzymes, antibodies, transcription factors, resistance proteins), Small molecules (e.g., plant hormones, signaling molecules, elicitors, chelators, osmoprotectants, phytoalexins, nucleoside analogs), Proteins (e.g., Bt toxins, pathogen recognition receptors, defensins, lectins), Metabolites and nutrients (e.g., amino acids, vitamins, cofactors, sugars), Biological control agents (e.g., microbial metabolites, quorum sensing inhibitors, volatile organic compounds), Hydrophilic pesticides and herbicides (e.g., phosphonic acid, glufosinate, streptomycin), Plant immunity modulators (e.g., salicylic acid, jasmonic acid, chitosan derivatives) These may be natural, recombinant, synthetic, or semi-synthetic. A functional agent may be associated with an NLP composition as described herein (e.g., loaded into or onto NLPs (e.g., encapsulated by, embedded in, or conjugated to NLPs)) to improve delivery and/or distribution of the functional agent to a plant, plant pest, plant symbiont, animal (e.g., human) pathogen, or animal pathogen vector) in accordance with the present compositions or methods. In some aspects, the functional agent is a polynucleotide (e.g., an mRNA, an siRNA, or a gRNA). In some aspects, the functional agent is a polypeptide. In some aspects, the functional agent is a small molecule. As used herein, the term “plant-modifying agent” refers to an agent that can alter the genetic properties (e.g., increase gene expression, decrease gene expression, or otherwise alter the nucleotide sequence of DNA or RNA), epigenetic properties, or biochemical properties of a plant in a manner that results in a change (e.g., increase or decrease) in plant fitness.

As used herein, a “hydrophilic core” refers to the internal aqueous compartment of a lipid nanoparticle that is capable of encapsulating and retaining hydrophilic cargo. In some embodiments, the hydrophilic core is enclosed by a phospholipid monolayer or bilayer. In some embodiments, the NLP is a unilamellar or multilamellar vesicle, micelle, or other lipid structure comprising a polar interior surrounded by lipids. The hydrophilic core may contain water, buffer, or other aqueous solvent and may solubilize nucleic acids, proteins, peptides, or small hydrophilic molecules.

As used herein, the term “formulated for delivery to a plant” refers to an NLP composition that includes an agriculturally acceptable carrier. As used herein, an “agriculturally acceptable” carrier is one that is suitable for use in agriculture without undue adverse side effects to the plants, the environment, or to humans or animals who consume the resulting agricultural products derived therefrom commensurate with a reasonable benefit/risk ratio. Non-limiting examples of agriculturally acceptable carriers or excipients are known in the art; see, e.g., the Compendium of Herbicide Adjuvants.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant parts, plant organs, plant tissues, seeds, plant cells, seeds, and progeny of the same. Plant cells include, without limitation, cells from seeds, suspension cultures, embryos, meristematic (e.g. meristem) regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue).

As used herein “encapsulated heterologous functional agent” refers to a heterologous functional agent that is enclosed in or incorporated in another structure, e.g., into an NLP as described herein. In some embodiments, there is no contact between the encapsulated heterologous functional agent with that what surrounds the NLP, e.g. water. In some embodiments, the encapsulated functional agent is provided in a hydrophilic liquid.

As used herein, the term “encapsulating” refers to the process of incorporating an agent into another entity, e.g., the process of incorporating heterologous functional agent (e.g., deltamethrin) into an NLP. As used herein, “unencapsulated heterologous functional agent” refers to a heterologous agent that is free in solution, e.g., dissolved or dispersed, and able to interact directly with its surroundings.

As used herein, the term “cellular uptake” refers to uptake of a NLP or a portion or component thereof (e.g., a heterologous functional agent carried by the NLP) by a cell, such as an animal cell, a plant cell, bacterial cell, or fungal cell. For example, uptake can involve transfer of the NLP or a portion of component thereof from the extracellular environment into or across the cell membrane, the cell wall, the extracellular matrix, or into the intracellular environment of the cell). Cellular uptake of NLPs may occur via active or passive cellular mechanisms. Cellular uptake includes aspects in which the entire NLP is taken up by a cell, e.g., taken up by endocytosis. In embodiments, one or more heterologous functional agents (e.g., polynucleotides, polypeptides, small molecule chemistries, etc.) are exposed to the cytoplasm of the target cell following endocytosis and endosomal escape. In some embodiments, an NLP (e.g., an NLP comprising a charged surface modifier (e.g., a polycarboxylate) has an increased rate of endosomal escape relative to an unmodified NLP. Cellular uptake also includes aspects in which the NLP fuses with the membrane of the target cell. In some embodiments, one or more heterologous functional agents (e.g., polynucleotides, polypeptides, small molecule chemistries, etc.) are exposed to the cytoplasm of the target cell following membrane fusion.

As used herein, the term “surface modifier” refers to a compound that is capable of modifying one or more surface characteristics of the NLP. In some embodiments, an NLP as described herein may comprise one or more surface modifiers affecting the surface characteristics of the NLP (e.g., the zeta potential of the NLP). In some embodiments, the surface modifier confers a positive charge to the NLP. In other embodiments, the surface modifier confers a negative charge to the NLP. In other embodiments, the surface modifier masks the charge of the NLP. In some embodiments, an NLP comprising a surface modifier (e.g., an NLP comprising a PEGylated lipid) has an increased rate of fusion with the membrane of the target cell (e.g., is more fusogenic) relative to an NLP not comprising a surface modifier. In some embodiments, the surface modifier increases the binding of an NLP to a plant or a plant part compared to an NLP not comprising the surface modifier. In some embodiments, the surface modifier increases the biodistribution of a heterologous functional agent comprised in the NLP upon contacting of a plant or plant part with the NLP as compared to the biodistribution of a heterologous agent comprised in an NLP not comprising the surface modifier.

As used herein, the term “stable NLP composition” (e.g., a composition including loaded or non-loaded NLPs) refers to an NLP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of NLPs (e.g., NLPs per mL of solution) relative to the number of NLPs in the NLP starting material (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or pesticidal and/or repellent activity) relative to the initial activity of the NLP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

As used herein, the term “untreated” refers to a plant, animal, fungus, or bacterium that has not been contacted with or delivered a NLP composition as described herein, including a separate plant, animal, fungus, or bacterium that has not been delivered the NLP composition, the same plant, fungus, or bacterium undergoing treatment assessed at a time point prior to delivery of the NLP composition, or the same plant, fungus, or bacterium undergoing treatment assessed at an untreated part of the plant, animal, fungus, or bacterium.

As used herein, the term NLP comprises a lipid structure (e.g., a liposome vesicle comprising a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter, and comprises at least one sterol, at least one synthetic cationic and/or at least one synthetic ionizable lipid, at least one phospholipid, and at least one polynucleotide. In some embodiments the NLP has a hydrophilic core and the hydrophilic core comprises the cargo (e.g. a polynucleotide cargo, a peptide cargo, a small molecule cargo).

As used herein, the term “polydispersity index” (PDI) is a unitless value derived from dynamic light scattering (DLS) measurements and is calculated based on the width of the particle size distribution. The PDI score of an NLP formulation reflects the uniformity of particle size distribution in the formulation. A lower PDI indicates a more monodisperse (uniform) population of particles, whereas a higher PDI reflects greater size heterogeneity.

As used herein, the term “synthetic quaternary ammonium salt lipid” refers to ionic compounds having a quaternary ammonium nitrogen, four alkyl or aryl groups connected to this nitrogen, and an anionic counter ion such as chloride or bromide. In some embodiments, QAS are positively-charged polyatomic ions of the structure [NR4]+, where R is an alkyl group, an aryl group or an organyl group. In some embodiments, among four alkyl groups, one is a long alkyl chain group containing more than eight hydrocarbons and also serves as the hydrophobic group. In some embodiments, QAS lipids are cationic lipids. Exemplary synthetic quaternary ammonium salt lipids include, but are not limited to SM-102 N-oxide, ALC-0315 N-oxide, Hexadecanedioic Acid Mono-L-carnitine Ester Chloride, Octadecanedioic Acid Mono-L-carnitine Ester Chloride, 14:0 TAP, 16:0 TAP, 18:0 TAP, DGTS (1,2-dipalmitoyl-sn-glycero-3-O-4′-[N,N,N-trimethyl]-homoserine), DC-6-14, 12:0 EPC, 14:0 EPC, 16:0 EPC, 18:0 EPC, 16:0-18:1 EPC, 1-Palmitoyl-2-arachidoyllecithin, iPhos-lipid1, 18:0 DDAB (DDAB), DOTAP, DOTMA, DODAC, DORI, DOBAQ, MVL5 and DOSPA. Structural formulas are recited, e.g., at www.broadpharm.com.

As used herein the term “tertiary amine lipid” refers to a lipid comprising NR3, where R is an alkyl group, an aryl group or an organyl group. In some embodiments, the tertiary amine is available for protonation, thus forming a positive charge. In some embodiments, the tertiary amine is ionizable. In some embodiments, a tertiary amine lipid is an ionizable lipid. Exemplary tertiary amine lipids include, but are not limited to: 113-O12B, 113-O16B, 14:0 DAP, 16:0 DAP, 1O14, 246C10, 304O13, 306-N16B, 306-O12B, 306-O12B-3, 306Oi10, 306Oi9-cis2, 4A3-SC8503O13, 80-O16B, 93-O17O, 93-O17S, 98N12-5, 9A1P9, 98N12-5, A12-Iso5-2DC18, A18-Iso5-2DC18, AA3-DLin, AA-T3A-C12, Al-28, ALC-0315 (ALC-315), ATX-001, ATX-100, BAMEA-O16B, C12-113, C12-200, C12-SPM, C13-112-tetra-tail, C13-112-tri-tail, C13-113-tetra-tail, C13-113-tri-tail, C14-4, C14-SPM, C3-K2-E14, cKK-E12, cKK-E12, cKK-E15, CL1, DLin-DMA, DLin-KC2-DMA (KC2), D-Lin-MC2-DMA, D-Lin-MC3-DMA (MC3), D-Lin-MC4-DMA, DODAP (18:1), DODMA, DOG-IM4, FTT5, G0-C14, GL67, IAJD249, IAJD93, IC8, iPhos-lipid2, iPhos-lipid3, iPhos-lipid4, L13, L14, L15, L16, L2, L3, L319, L9, Lipid 10, Lipid 14, Lipid 16, Lipid 2, Lipid 23, Lipid 29, Lipid 5, Lipid 8, Lipid A4, Lipid A6, Lipid A9, Lipid AX4, Lipid C24, Lipid Catechol, Lipid III-45, Lipid R6, LP01, MVL5, NT1-O14B, OC2-K3-E10, OF-02, OF-C4-Deg-Lin, OF-Deg-Lin, PPZ-A10, RCB-4-8, RM 133-3, RM 137-15, SM-102, SSPalmM, SSPalmO-Phe, TCL053, T3, YK-009, YSK05, ZA3-Ep10, and selected BP lipids. In some embodiments, the ionizable lipid is a charged lipid, e.g. a positively charged lipid or a negatively charged lipid. In some embodiments, the ionizable lipid is not a charged lipid. Structural formulas are recited, e.g., at www.broadpharm.com.

As used herein, the term “cationic lipid” refers to lipids (e.g., a lipid or a lipidoid, e.g., a synthetic lipid or lipidoid) with head groups bearing permanent positive charges. A cationic lipid is a positively charged lipid. Exemplary cationic lipids are quaternary ammonium salt lipids.

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH). These ionizable lipids usually contain amino head groups and have an acid dissociation constant (pKa) less than 7; thus, they are protonated and positively charged at acidic pH (pH<6.0), neutral at physiological condition (around pH=7.4), and negatively charged at higher pH values (e.g., pH 10). In some embodiments, the ionizable lipid is a zwitterionic lipid, that is a lipid that is electrically neutral and carries no net charge.

As used herein, the term “a chemically synthesized lipid” refers to a lipid that does not occur naturally in nature.

As used herein, the term “a natural lipid” refers to a lipid that occurs naturally in nature.

Exemplary natural lipids are PS, PC, PA, DOPE, and DSPC. In some embodiments, the at least one natural phospholipid in the NLPs is derived from lecithin. Lecithin is described in the United States Pharmacopoeia (USP) as a complex mixture of acetone-insoluble phosphatides, which consists chiefly of PC, PE, phosphatidylserine, and phosphatidylinositol, combined with various amounts of other substances such as triglycerides, fatty acids, and carbohydrates, as separated from the crude vegetable oil source.

As used herein, the term “de-oiled sunflower lecithin” and the term “de-oiled soybean lecithin”, refer to a sunflower lecithin extract, and a soybean lecithin extract, respectively, from which the oil fraction has been removed. In some embodiments, de-oiled sunflower lecithin and de-oiled soybean lecithin are enriched in phospholipids relative to non-phospholipid constituents.

As used herein, the term “lipidoid” refers to a molecule having one or more characteristics of a lipid.

As used herein, the term “stable NLP composition” (e.g., a composition including loaded or non-loaded NLPs) refers to a NLP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of NLPs (e.g., NLPs per ml of solution) relative to the number of NLPs in the NLP composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or pesticidal and/or repellent activity) relative to the initial activity of the NLP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

As used herein, the term “pest” refers to organisms that cause damage to plants or other organisms, are present where they are not wanted, or otherwise are detrimental to humans, for example, by negatively impacting human agricultural methods or products. Pests may include, for example, invertebrates (e.g., insects, nematodes, or mollusks), microorganisms (e.g., phytopathogens, endophytes, obligate parasites, facultative parasites, or facultative saprophytes), such as bacteria, fungi, oomycetes, or viruses; or weeds.

As used herein, the term “pesticidal agent” or “pesticide” refers to an agent, composition, or substance therein, that controls or decreases the fitness (e.g., kills or inhibits the growth, reduces fecundity, proliferation, division, reproduction, or spread) of an agricultural, environmental, or domestic/household pest, such as an insect, mollusk, nematode, fungus, bacterium, weed, or virus. Pesticides are understood to encompass naturally occurring or synthetic insecticides (larvicides or adulticides), insect growth regulators, acaricides (miticides), molluscicides, nematicides, ectoparasiticides, bactericides, fungicides, or herbicides. The term “pesticidal agent” may further encompass other bioactive molecules such as antibiotics, antivirals pesticides, antifungals, antihelminthics, nutrients, and/or agents that stun or slow insect movement.

### I. NLP Compositions

### A. Physicochemical Properties

### i) Structure

In some embodiments, the NLP is a lipid (e.g., lipid bilayer, unilamellar, or multilamellar structure) structure. NLPs refer to an enclosed lipid-bilayer structure, that is about 5-2000 nm in diameter. In some embodiments, NLPs comprise phospholipids that are organized into one or more phospholipid layers (e.g., a phospholipid monolayer, a phospholipid bilayer, etc.), to form a liposome. In some embodiments, the liposomes can be any one or combination of vesicles selected from the group consisting of small unilamellar vesicles (SUV), large unilamellar vesicles (LUV), multilamellar vesicles (MLV), multivesicular vesicles (MVV), large multivesicular vesicles ((LMVV), also referred to, at times, by the term giant multivesicular vesicles, (“GMV”), oligolamellar vesicles (OLV), and others. In some embodiments, the phospholipids form a micellar structure with a hydrophilic core surrounded by a phospholipid bilayer.

In some embodiments, the NLP comprises a hydrophilic core. Such structures may include unilamellar or multilamellar vesicles where an aqueous core is enclosed within a phospholipid bilayer. In some instances, micellar or pseudo-micellar structures with a polar interior may also be formed. Hydrophilic cores are especially well-suited for the encapsulation of water-soluble agents such as nucleic acids, peptides, proteins, dyes, salts, and small polar molecules. Hydrophilic core NLPs may improve cargo stability and protect against enzymatic degradation.

### ii) Size

In some embodiments, the NLPs are about 5-1000 nm in diameter. For example, in some embodiments, the NLP has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the NLP has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the NLP has a mean diameter of about 50-200 nm. In certain instances, the NLP has a mean diameter of about 50-300 nm. In certain instances, the NLP has a mean diameter of about 200-500 nm. In certain instances, the NLP has a mean diameter of about 30-150 nm. In some instances, the NLP has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the NLP has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the NLP.

In some instances, the NLP has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the NLP has a mean volume of 65 nm³ to 5.3×10¹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁹-5.3×10⁸ nm³). In some instances, the NLP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, NLP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

iii) Zeta Potential

The NLP composition comprising a plurality of modified NLPs comprising a synthetic charged lipid may have, e.g., a zeta potential of greater than −30 mV when in the absence of cargo, greater than −20 mV, greater than −5 mV, greater than 0 mV, or about 30 my when in the absence of cargo. In some examples, the NLP composition has a negative zeta potential, e.g., a zeta potential of less than 0 mV, less than −10 mV, less than −20 mV, less than −30 mV, less than −40 mV, or less than −50 mV when in the absence of cargo. In some examples, the NLP composition has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the NLP composition has a zeta potential of about 0.

The zeta potential of the NLP composition may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### B. Lipid Composition

In some embodiments, the NLPs comprise (i) at least one sterol, (ii) at least one cationic lipid and/or at least one synthetic ionizable lipid; (iii) at least one phospholipid; and (iv) at least one polynucleotide. In some embodiments, the polynucleotide is an mRNA or an siRNA. In some embodiments, the NLPs further comprise a surface modifier (e.g. a pegylated lipid). In some embodiments, the NLPs comprises a hydrophobically modified pectin derivative. In some embodiments, the NLPs comprises a boron-containing lipids. In some embodiments, the NLP composition further comprises one or more heterologous functional agents. In some embodiments, the heterologous functional agent is a hydrophylic agent. In some embodiments, the hydrophylic heterologous functional agent is nuclease inhibitor. In some embodiments the NLP composition further comprises a dye.

In some embodiments, the composition of lipids, including zwitterionic phospholipids such as DSPC, DOPE, or lecithins, in combination with sterols (e.g., cholesterol) and ionizable or cationic lipids, enables the formation of NLPs with stable hydrophilic cores. In some embodiments, the NLP includes a surface modifier (e.g., PEGylated lipid) to further stabilize the aqueous core.

### i) Phospholipids (PL)

Several embodiments relate to an NLP composition comprising at least one phospholipid. In some embodiments, the phospholipids in the NLP form one or more phospholipid layers (e.g., a phospholipid monolayer, a phospholipid bilayer, etc.). In some embodiments, the phospholipids form a micellar structure with a hydrophilic core surrounded by a phospholipid bilayer. In some embodiments, the NLP comprises several layers of phospholipid layers. In some embodiments, the NLPs are sealed structure in the micron and submicron range dispersed in an aqueous solution. In some embodiments, NLPs comprise one or more bilayers (lamellae) separating the external aqueous solution from the internal phase, or the “core”. In some embodiments, the core is hydrophobic. In other embodiments, the core is hydrophilic. In some embodiments, the one or more phospholipid layers (e.g., a monolayer, a bilayer, etc.) comprises one or more amphipathic agents. Amphipathic agents comprise both polar and apolar regions. When amphipathic agents are present in an aqueous phase, they self-aggregate such that their hydrophilic moiety faces the aqueous phase, while their hydrophobic domain is “protected” from the aqueous phase. In some embodiments, an NLP may comprise a phospholipid bilayer wherein the hydrophobic domains face each other, and the hydrophilic head groups face the hydrophilic core of the NLP. In some embodiments, NLPs are formed by organizing amphipathic agents, e.g., phospholipids, in a lamellar phase wherein the lamellae form closed structures and organize into vesicles.

Several embodiments relate to an NLP composition used as a carrier to facilitate uptake and biodistribution of a functional agent (e.g. an mRNA or an siRNA) through a plant. In some embodiments, an NLP composition comprising two or more types of liposomes are used to facilitate uptake and biodistribution.

Several embodiments relate to NLP compositions comprising at least one phospholipid, at least one of which is a liposome forming phospholipid. Without being limited by theory, the amount of phospholipids in the NLP can be determined as organic phosphorous by the modified Bartlett method (Shmeeda H, Even-Chen S, Honen R, Cohen R, Weintraub C, Barenholz Y. 2003. Enzymatic assays for quality control and pharmacokinetics of liposome formulations: comparison with nonenzymatic conventional methodologies. Methods Enzymol 367:272-92).

In some embodiments, the NLP compositions comprise at least one phospholipid selected from glycerophospholipids and sphingomyelins. The glycerophospholipids have a glycerol backbone wherein at least one, preferably two, of the hydroxyl groups at the head group is substituted by one or two hydrocarbon tails (chains), typically, an acyl, alkyl or alkenyl tails, and the third hydroxyl group is substituted by a phosphate (phosphatidic acid) or a phospho-ester such as phosphocholine group (as exemplified in phosphatidylcholine), being the polar head group of the glycerophospholipid or combination of any of the above, and/or derivatives of same and may contain a chemically reactive group (such as an amine, acid, ester, aldehyde or alcohol). Examples of glycerophospholipids include, but are not limited thereto, phosphatidylglycerols (PG) including dimyristoyl phosphatidylglycerol (DMPG); phosphatidylcholine (PC), including egg yolk phosphatidylcholine, soybean PC, sunflower PC, rapeseed PC, krill PC, canola PC, flax seed lecithin, wheat lecithin, dimyristoyl phosphatidylcholine (DMPC, Tm 24° C.), 1-palmitoyl-2-oleoylphosphatidyl choline (POPC), hydrogenated soy phosphatidylcholine (HSPC, Tm 65° C.), distearoylphosphatidylcholine (DSPC, Tm 55° C.); di-lauroyl-sn-glycero-2phosphocholine (DLPC); 1,2-dipalmitoyl-sn-glycero-3-phosphocholine (DPPC, Tm 41° C.); 1,2-dinonadecanoyl-sn-glycero-3-phosphocholine; 1,2-diarachidoyl-sn-glycero-3-phosphocholine (DBPC); 1,2-dihenarachidoyl-sn-glycero-3-phosphocholine; 1,2-dibehenoyl-sn-glycero-3-phosphocholine 1,2-ditricosanoyl-sn-glycero-3-phosphocholine 1,2-dilignoceroyl-sn-glycero-3-phosphocholine; 1-myristoyl-2-stearoyl-sn-glycero-3-phosphocholine; 1-palmitoyl-2-stearoyl-sn-glycero-3-phosphocholine (PSPC); 1-stearoyl-2-palmitoyl-sn-glycero-3-phosphocholine (SPPC); 1,2-di-oleoyl-sn-glycero-3-phosphocholine (DOPC-1.7° C.); phosphatidic acid (PA), phosphatidylinositol (PI), phosphatidylserine (PS), phosphatidylethanolamine (PE), e.g. 1,2-dioleoyl-sn-glycero-3-phosphoethanolamine (18:1 (A9-Cis) PE (DOPE)). The sphingomyelins consist of a ceramide (N-acyl sphingosine) unit having a phosphocholine moiety attached to position 1 as the polar head group. The term “sphingomyelin” or “SPM” as used herein denotes any N-acetyl sphingosine conjugated to a phosphocholine group, the later forming the polar head group of the sphingomyelin (N-acyl sphingosyl phospholcholines). The acyl chain bound to the primary amino group of the sphingosine (to form the ceramide) may be saturated or unsaturated, branched or unbranched.

In some embodiments, an NLP composition comprises a phospholipid having one or two C14 to C24 hydrocarbon tails (e.g., acyl, alkyl or alkenyl chain) with varying degrees of saturation, from being fully saturated to being fully, partially or non-hydrogenated lipids. In some embodiments, phospholipids may be further converted to saturated phospholipids by means of hydrogenation or further treated with enzymes to, e.g., remove partially fatty acids (e.g. using phospholipase A2) or to convert a polar head group (e.g. using phospholipase D). In some embodiments, an unsaturated natural phospholipid is converted to a saturated phospholipid.

In some embodiments, the NLP composition comprises at least one phospholipid comprising a polar head group. In some embodiments, the polar head group comprises an alcohol moiety. In some embodiments, the polar head group is one comprising a serine moiety. In some embodiments, the polar head group is one comprising a choline moiety. In some embodiments, the polar head group is one comprising ethanolamine. In some embodiments, the polar head group is one comprising glycerol.

In some embodiments, an NLP composition comprises at least one phospholipid comprising a polar inositol head group. In some embodiments, the phospholipid comprising an inositol head group is selected from the group consisting of phosphatidylinositol (PI), PI(4)P, PI(3)P, PI(3,4,5)P3, PI(4,5)P2, PI(3,5)P2, and PI(3,4)P2. In some embodiments, at least one phospholipid has an acidic head group. In some embodiments, the acidic head group comprises a moiety selected from the group consisting of glycerol, hydroxyl, carboxyl, amine, and phosphoric group.

In some embodiments, an NLP composition comprises at least one phospholipid that is an acidic phospholipid. Acidic phospholipids include phosphatidylglycerols (PGs) such as dilauroylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), egg yolk phosphatidylglycerol (egg yolk PG), hydrogenated egg yolk phosphatidylglycerol; phosphatidylinositols (PIs) such as phosphatidylinositol, dimyristoylphosphatidylinositol, dipalmitoylphosphatidylinositol (DPPI), distearoylphosphatidylinositol (DSPI), dioleoylphosphatidylinositol (DOPI), soybean phosphatidylinositol (soybean PI), hydrogenated soybean phosphatidylinositol, phosphoinositides, sphingomyelin and phosphatidic acid. Each of these acidic phospholipids can be used alone or in combination of two or more in the NLPs of the presented disclosure.

In some embodiments, the at least one phospholipid in the NLPs is derived from lecithin. Lecithin is described in the United States Pharmacopoeia (USP) as a complex mixture of acetone-insoluble phosphatides, which consists chiefly of PC, PE, phosphatidylserine, and phosphatidylinositol, combined with various amounts of other substances such as triglycerides, fatty acids, and carbohydrates, as separated from the crude vegetable oil source.

In some embodiments, about 5%-50% (w/w) of the lipids in an NLP composition is phospholipid (e.g., about 10%-20% of the lipids in an NLP composition is phospholipid, e.g., about 10%, 12.5%, 16%, or 20% of the lipids in an NLP composition is phospholipid). In some embodiments, about 30%-75% (e.g., about 35% or about 50% phospholipid) of the lipids in an NLP composition is phospholipid. In some embodiments, about 35%-50% (e.g., about 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%) of the lipids in an NLP composition is phospholipid.

In some embodiments, about 5%-50% (w/w) of the lipids in an NLP composition is phospholipid (e.g., about 10%-20% of the lipids in an NLP composition is phospholipid, e.g., about 10%, 12.5%, 16%, or 20% of the lipids in an NLP composition is phospholipid). In some embodiments, about 30%-75% (e.g., about 35% or about 50% phospholipid) of the lipids in an NLP composition is phospholipid. In some embodiments, about 35%-50% (e.g., about 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%) of the lipids in an NLP composition is phospholipid.

### ii) Synthetic Lipids

In some embodiments, the NLP comprises at least one synthetic lipid. In some embodiments, the at least one synthetic lipid is a quaternary ammonium salt lipid. In some embodiments, the at least one synthetic lipid is a tertiary amine lipid, that is a lipid comprising a tertiary amine group. In some embodiments, the NLP comprises at least 1, 2, 3, 4, 5, 6, 7, 8, 9, 10, 20, 30 or 50 different quaternary ammonium salt lipids. In some embodiments, the NLP comprises at least 1, 2, 3, 4, 5, 6, 7, 8, 9, 10, 20, 30 or 50 different tertiary amine lipids. In some embodiments, the NLPs comprise at least one quaternary ammonium salt lipid and at least one tertiary amine lipid.

Exemplary synthetic quaternary ammonium salt lipids include, but are not limited to SM-102 N-oxide, ALC-0315 N-oxide, Hexadecanedioic Acid Mono-L-carnitine Ester Chloride, Octadecanedioic Acid Mono-L-carnitine Ester Chloride, 14:0 TAP, 16:0 TAP, 18:0 TAP, DGTS (1,2-dipalmitoyl-sn-glycero-3-O-4′-[N,N,N-trimethyl]-homoserine), DC-6-14, 12:0 EPC, 14:0 EPC, 16:0 EPC, 18:0 EPC, 16:0-18:1 EPC, 1-Palmitoyl-2-arachidoyllecithin, iPhos-lipid1, 18:0 DDAB (DDAB), DOTAP, DOTMA, DODAC, DORI, DOBAQ, MVL5, DOSPA, and analogs thereof. Structure formulas are recited, e.g., at www.broadpharm.com.

In some embodiments, the synthetic lipid is a tertiary amine lipid. Tertiary amine lipids include, but are not limited to 113-O12B, 113-O16B, 14:0 DAP, 16:0 DAP, 1O14, 246C10, 304O13, 306-N16B, 306-O12B, 306-O12B-3, 306Oi10, 306Oi9-cis2, 4A3-SC8503O13, 80-O16B, 93-O17O, 93-O17S, 98N12-5, 9A1P9, 98N12-5, A12-Iso5-2DC18, A18-Iso5-2DC18, AA3-DLin, AA-T3A-C12, Al-28, ALC-0315 (ALC-315), ATX-001, ATX-100, BAMEA-O16B, C12-113, C12-200, C12-SPM, C13-112-tetra-tail, C13-112-tri-tail, C13-113-tetra-tail, C13-113-tri-tail, C14-4, C14-SPM, C3-K2-E14, cKK-E12, cKK-E12, cKK-E15, CL1, DLin-DMA, DLin-KC2-DMA (KC2), D-Lin-MC2-DMA, D-Lin-MC3-DMA (MC3), D-Lin-MC4-DMA, DODAP (18:1), DODMA, DOG-IM4, FTT5, G0-C14, GL67, IAJD249, IAJD93, IC8, iPhos-lipid2, iPhos-lipid3, iPhos-lipid4, L13, L14, L15, L16, L2, L3, L319, L9, Lipid 10, Lipid 14, Lipid 16, Lipid 2, Lipid 23, Lipid 29, Lipid 5, Lipid 8, Lipid A4, Lipid A6, Lipid A9, Lipid AX4, Lipid C24, Lipid Catechol, Lipid III-45, Lipid R6, LP01, MVL5, NT1-O14B, OC2-K3-E10, OF-02, OF-C4-Deg-Lin, OF-Deg-Lin, PPZ-A10, RCB-4-8, RM 133-3, RM 137-15, SM-102, SSPalmM, SSPalmO-Phe, TCL053, T3, YK-009, YSK05, ZA3-Ep10, selected BP lipids, and analogs thereof. Structure formulas are recited, e.g., at www.broadpharm.com. In some embodiments, the ionizable lipid is a charged lipid, e.g. a positively or a negatively charged lipid. In some embodiments, the ionizable lipid is not a charged lipid.

Exemplary lipid chemical formulas are provided by 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), dioleoyl-3-trimethylammonium propane (DODAP), [(6Z,9Z,28Z,31Z)-Heptatriaconta-6,9,28,31-tetraen-19-yl 4-(dimethylamino)butanoate](DLin-MC3-DMA (MC3)), 18:1 DAP 1,2-dioleoyl-3-dimethylammonium-propane (DODAP), 8-[(2-hydroxyethyl)[6-oxo-6-(undecyloxy)hexyl]amino]-octanoic acid, 1-octylnonyl ester (SM-102) and 6-((2-hexyldecanoyl)oxy)-N-(6-((2-hexyldecanoyl)oxy)hexyl)-N-(4-hydroxybutyl)hexan-1-aminium (ALC-315).

iii) Sterol

In some embodiments, an NLP composition comprises one or more lipids that do not spontaneously vesiculate yet can be incorporated into vesicles. Non-limiting examples of non-vesiculating lipids include, sterols, sphingolipids (e.g., sphingomyelin), lipoproteins. In some embodiments, the NLP composition comprises one or more sterols selected from the group consisting of β-sitosterol, β-sitostanol, stigmasterol, stigmastanol, campesterol, campestanol, ergosterol, avenasterol, brassicasterol, fucosterol, cholesterol, cholesteryl hemisuccinate, and cholesteryl sulfate any combination of two or more of these sterols. In some embodiments, the sterol is a plant derived sterol (e.g., phytosterol). In the NLP composition comprises one or more phytosterols selected from the group consisting of β-sitosterol, β-sitostanol, stigmasterol, stigmastanol, campesterol, campestanol, ergosterol, avenasterol, brassicasterol and any combination of two or more of these sterols. In some embodiments, the NLP composition comprises one or more phytosterols selected from the group consisting of β-sitosterol, stigmasterol, and ergosterol. In some embodiments, the sterol is cholesterol. In some embodiments, the sterol is sitosterol.

In some instances, the NLPs comprise an exogenous sterol, e.g., sitosterol, sitostanol, 3-sitosterol, 7a-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, an NLP composition comprises one or more natural plant derived non-polar lipids obtained from vegetable sources like, e.g., seed oil (from soybeans, rape (canola), wheat germ, sunflower, flax, cotton, corn, coconut, arachis, sesame), pulp oil (palm, olive, avocado pulp), desert shrub, tobacco, bean, and carrot. In some embodiments, the non-polar lipids comprise triglycerides that typically each comprise at least one fatty acid selected from the group consisting of C6:0, C8:0, C10:0, C12:0, C14:0, C15:0, C16:0, C17:0, C18:0, C20:0, C22:0, and C24:0, saturated fatty acids are selected from C16:1 (n-7), C16:1 (n-9), C17:1 (n-7), C18:1 (n-7), C20:1 (n-7), C20:1 (n-9), C22: (n-9) and C24:1 (n-9), and mono-unsaturated fatty acids C18:2 (n-6), C18:3 (n-3), C18:3 (n-6), C18:4 (n-3), C20:2 (n-6), C20:3 (n-6), C20:4 (n-6), C20:5 (n-3), C22:2 (n-6), and C22:4 (n-6). The types of fatty acid profiles of 80 vegetable oils are described by Dubois et al., Eur. J. Lipid Sci. Technol. 109 (2007) 710-732, which is incorporated herein by reference.

In some embodiments, an NLP composition comprises a non-polar lipid comprising 40% of at least one fatty acid chain selected from the group consisting of a poly-unsaturated fatty acid; a mono-unsaturated fatty acid, and a saturated fatty acid. In some embodiments, an NLP composition comprises at least 1%, 5%, 10%, 15%, 20%, 25%, 30%, 35%, 40%, 45%, 50%, 55%, 60%, or more than 60% (w/w) oil (e.g., soy bean oil), e.g., 1%-10%, 10%-20%, 20%-30%, 30%-40%, 40%-50%, or 50%-60% (w/w) soybean oil. In some embodiments, an NLP composition comprises a molar ratio of about 35%-50% oil (e.g., soybean oil), e.g., about 36%, 38.5%, 42.5%, or 46.5% oil. In some embodiments, an NLP composition comprises about 20%-60% oil.

### iv) Pegylated Lipids

In some embodiments, the NLP comprises one or more PEGylated lipids. Polyethylene glycol (PEG) length can vary from 1 kDa to 10 kDa. In some embodiments, an NLP composition comprising one or more PEGylated lipid having a PEG length of 2 kDa. In some embodiments, an NLP composition comprises one or more the PEGylated lipids independently selected from C14-PEG2k, C18-PEG2k, and DMPE-PEG2k. In some embodiments the PEGylated lipid is a PEG5K PEGylated lipid (e.g. C14-PEG5k, C18-PEG5k or DMPE-PEG5K). In some embodiments, an NLP composition comprises a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% of one or more PEGylated lipids (e.g., C14-PEG2k, C18-PEG2k, C18-PEG5K, DMPE-PEG2k, etc.). In some embodiments, an NLP composition comprises a molar ratio of at least 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% of one or more PEGylated lipids. In some embodiments, an NLP composition comprises about 0.1%-10% (w/w) PEGylated lipid (e.g., C14-PEG2k, C18-PEG2k, DMPE-PEG2k, etc.). In some embodiments, an NLP composition comprises about 1%-3% of one or more PEGylated lipids. In some embodiments, an NLP composition comprises about 1.5% of one or more PEGylated lipids. In some embodiments, an NLP composition comprises about 2.5% of one or more PEGylated lipids. In some embodiments, an NLP composition comprising one or more PEGylated lipids has an altered plant uptake relative to an NLP composition not comprising the one or more PEGylated lipids.

In some embodiments, an NLP composition comprises one or more PEGylated lipids. In some embodiments, about 5%-50% (w/w) of the lipids in an NLP composition is PEGylated lipid (e.g., about 10%-20% of the lipids in an NLP composition is PEGylated lipid, e.g., about 10%, 12.5%, 16%, or 20% of the lipids in an NLP composition is PEGylated lipid). In some embodiments, about 30%-75% (e.g., about 35% or about 50% PEGylated lipid) of the lipids in an NLP composition is PEGylated lipid. In some embodiments, about 35%-50% (e.g., about 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%) of the lipids in an NLP composition is PEGylated lipid.

In some embodiments, an NLP composition comprising one or more PEGylated lipids has enhanced uptake relative to an NLP composition not comprising the one or more PEGylated lipids. In some embodiments, an NLP composition comprising one or more PEGylated lipids has altered stability (e.g., increased stability, decreased stability, etc.) relative to an NLP composition that not comprising the one or more PEGylated lipids. In some embodiments, an NLP composition comprising one or more PEGylated lipids has altered particle size relative to an NLP composition not comprising the one or more PEGylated lipids. In some embodiments, an NLP composition comprising one or more PEGylated lipids is less likely to be phagocytosed by a cell than an NLP composition not comprising the one or more PEGylated lipids. In some embodiments, an NLP composition comprises one or more surface modifiers comprising one or more PEG moieties having a molecular weight of the head group from about 750 Da to about 20,000 Da. In some embodiments, an NLP composition comprises one or more surface modifiers comprising one or more PEG moieties having a molecular weight of the head group from about 750 Da to about 12,000 Da. In some embodiments, an NLP composition comprises one or more surface modifiers comprising one or more PEG moieties having a molecular weight of the head group between about 1,000 Da to about 5,000 Da. In some embodiments, an NLP composition comprises one or more neutral (uncharged) lipopolymers. In some embodiments, an NLP composition comprises one or more positively charged lipopolymers. In some embodiments, an NLP composition comprises one or more negatively charged lipopolymers. In some embodiments, an NLP composition comprises one or more neutral distearoyl glycerol and the negatively charged distearoyl phosphatidylethanolamine, both covalently attached to methoxy poly(ethylene glycol) (mPEG or PEG) of Mw 750, 2000, 5000, or 12000.

In some embodiments, pegylated lipid moieties act as surface modifiers. In some embodiments, a surface modifier stabilizes an NLP composition. An NLP composition as described herein may comprise (e.g., be loaded with, encapsulate, be conjugated to) or be formulated with (e.g., be suspended or resuspended in a solution comprising) one or more surface modifiers. In some embodiments, the surface modifier is a pegylated lipid. In some embodiments, the pegylated lipid is selected from the group consisting of PEG5K PE 14:0, PEG2K PE 14:0, and PEG2K-PE 18:0

In some embodiments, the one or more surface modifiers that affect uptake and biodistribution of an NLP in a plant. In some embodiments, the surface modifier is a synthetic compound, e.g. a synthetic lipid. In some embodiments, the surface modifier alters one or more surface characteristics of an NLP composition. In some embodiments, a surface modifier structurally alters the NLP, e.g., by adding a chemical group to the exterior surface of the NLP. In some embodiments, an NLP composition comprises a glycolipid moiety exposed at the external surface of the NLP composition. In some embodiments, an NLP composition comprises at least one glycoprotein embedded in the outer surface of the NLP composition. In some embodiments, at least a portion of the surface modifier is integrated into a lipid membrane of the NLP, e.g., a lipoid domain that is embedded into the phospholipid membrane. In some embodiments, at least a portion of the surface modifier is exposed to the outside of the NLP, facing e.g., the air, the soil, or a solution in which the NLPs are dispersed. In some embodiments, the surface modifier is an emulsifier. In some embodiments, the surface modifier is amphipathic in nature, e.g., comprises a hydrophobic part and a hydrophilic part chemically connected in one molecule. In some embodiments, the surface modifier is a surfactant. In some embodiments, the surface modifier affects the surface charge of an NLP, e.g., by making the surface charge of an NLP more or less negative in charge. In some embodiments, the surface modifier confers a positive charge to the NLP. In other embodiments, the surface modifier confers a negative charge to the NLP. In other embodiments, the surface modifier masks the charge of the NLP.

In some embodiments, one or more surface modifiers, e.g. pegylated lipids, increase uptake of the NLP composition by a plant or plant part (e.g., root, leaf, plant cell, etc.). In some embodiments, the one or more surface modifiers increase the uptake of the NLP composition as a whole. In some embodiments, the one or more surface modifiers increase the uptake of a portion or component of the NLP composition, such as the uptake of a heterologous functional agent (e.g., a heterologous agricultural agent (e.g., pesticidal agent, fertilizing agent, herbicidal agent, plant-modifying agent, plant growth promoting agent, biostimulants, or plant immunity elicitors) carried by the NLP.

### v) Pectins

In some embodiments, the NLPs comprise one or more pectins. Pectins are a kind of complex linear polymer polysaccharide. In some embodiments, hydrophobically modified pectin derivatives are obtained, e.g. by modification of pectin with di-acyl chlorides (glutaryl and sebacoyl chloride). In some embodiments, the incorporation of hydrophobic pectin in NLPs enhances colloidal stability and takes advantage of natural plant pectin transporters. In other embodiments, nucleic acids are encapsulated with pectin or hydrophobic pectin by the addition of calcium ions for via cross linking interactions. In some embodiments, incorporation of pectin enhances the delivery of a bioactive to a plant. In some embodiments, the pectins comprised in the rNLPs are as described in Kedir et al., Heliyon 16; 8(9):e10654 (2022) and Opanasopit et al., AAPS PharmSciTech. 9(1): 67-74 (2008). In some embodiments, the pectins are hydrophobically modified pectin derivatives, e.g. by reacting with glutaryl and sebacoyl chloride, are described by Seslija et al., Int. J. Biol. Macromol. 113, 924-932 (2018). In some embodiments, pectins for producing NLPs comprise hydrophobic alkyl chains-pectin conjugates are described by Miralles-Houzelle et al., Langmuir 17(5), 1384-1391 (2001).

### vi) Boron-Containing Lipids

In some embodiments, the NLPs comprise boron-containing lipids. In some embodiments, boron-containing lipids impart plant responsiveness. In some embodiments, boronic acid moieties are incorporated into the NLP lipid structure. In some embodiments boronic acid-modified lipids interact with cell wall polysaccharides in a reversible manner, allowing transport through the cell wall. After the NLP transverses the cell wall the boron lipid will then enhance NLP transport into the cell via the plant membrane boron transporter. In some embodiments, the unique combination of pH responsiveness and cone shape allows targeted gene regulation in plants. In some embodiments, boronic acid comprised in the NLPs are as described in Zhang et al., Chem Commun (Camb), 54(48): 6169-6172. (2018) or Qualls et al., Chembiochem 23(21):e202200402 (2022). Cell-selective messenger RNA delivery in plants is conducted by modulating the interface of phenylboronic acid-derived lipid nanoparticles and cellular surface sialic acid as reported by Tang et al., ACS Appl Mater Interfaces, 11(50):46585-46590 (2019).

vii) Surface Modifiers

Several embodiments relate to an NLP composition comprising at least one surface modifier. In some embodiments, the surface modifier stabilizes an NLP composition. In some embodiments, the surface modifier is an emulsifier. An NLP composition as described herein may comprise (e.g., be loaded with, encapsulate, be conjugated to) or be formulated with (e.g., be suspended or resuspended in a solution comprising) one or more surface modifiers. In some embodiments, the surface modifier affects the binding of any of the constituents of the NLP composition to any plant part. In some embodiments, one or more surface modifiers are integrated into one or more of the phospholipid layers of the NLP. In some embodiments, an NLP composition comprises at least one surface modifier. In some embodiments, an NLP composition comprises at least two, three, four, five or more surface modifiers. Exemplary surface modifiers are described in PCT/US2024/015064, which is incorporated in its entirety herein.

In some embodiments, an NLP composition comprises at least 1%, 5%, 10%, 20%, 30%, 40%, 50%, 60%, 70%, 80%, 90%, or more than 90% of one or more surface modifiers. In some embodiments, an NLP composition comprises a weight/weight ratio of at least 0.1%, 1%, 5%, 10%, 15%, 20%, 25%, 30%, 35%, 40%, 45%, 50%, 55%, 60%, 65%, 70%, 75% 80%, 85%, 90%, or more than 90% of a synthetic chemical surface modifier (e.g., a pegylated compound, a polycarboxylate, etc.). In some embodiments, an NLP composition comprises a weight/weight ratio of at least 1%-10%, 10%-20%, 20%-30%, 30%-40%, 40%-50%, 50%-60%, 60%-70%, 70%-80%, or 80%-90% of one or more surface modifiers (e.g., a polycarboxylate). In some embodiments, an NLP composition comprises a weight/weight ratio of at least about 30%-75% of a polycarboxylate surface modifier. In some embodiments, the NLPs contain up to 5 mole % surface modifier. In some embodiments, an NLP composition comprises between about 0.1 mole % to 5 mole %, between about 0.5 mole % to 4 mole %, between about 1 mole % to 3 mole % of one or more surface modifiers. In some embodiments, the surface modifier is a pegylated lipid.

In some embodiments the presence of a surface modifier in the NLP increases the uptake of the NLP composition by a plant or plant part (e.g., root, leaf, plant cell, etc.). In some embodiments, the one or more surface modifiers increase the uptake of the rNLP composition as a whole. In some embodiments, the one or more surface modifiers increase the uptake of a portion or component of the NLP composition, such as the uptake of a heterologous functional agent (e.g., a heterologous agricultural agent (e.g., RNA or siRNA) carried by the NLP. The degree to which NLP uptake is increased by the surface modifier may vary depending on the plant or plant part to which the NLP composition is delivered. In some embodiments, one or more surface modifiers may increase uptake of an NLP composition by a plant or plant part by at least 1%, 2%, 5%, 10%, 15%, 20%, 30%, 40%, 50%, 60%, 70%, 80%, 90%, or 100% relative to an NLP composition lacking the one or more surface modifiers. In some embodiments, one or more surface modifiers may increase uptake of an NLP composition by a plant or plant part by at least 2×-fold, 4×-fold, 5×-fold, 1Ox-fold, 100×-fold, or 1000×-fold relative to an NLP composition lacking the one or more surface modifiers. In some embodiments, an NLP composition comprises at least one surface modifier that increases uptake of the NLP composition in a plant or plant part. In some embodiments, an NLP composition comprises at least two, three, four, five or more surface modifiers that increases uptake of the NLP composition in a plant or plant part.

In some embodiments, the surface modifier is a PEGylated lipid. Polyethylene glycol (PEG) length can vary from 1 kDa to 10 kDa. In some embodiments, an NLP composition comprising one or more PEGylated lipid having a PEG length of 2 kDa. In some embodiments, an NLP composition comprises one or more the PEGylated lipids independently selected from C14-PEG2k, C18-PEG2k, and DMPE-PEG2k. In some embodiments the PEGylated lipid is a PEG5K PEGylated lipid (e.g. C14-PEG5k, C18-PEG5k or DMPE-PEG5K). In some embodiments, an NLP composition comprises a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% of one or more PEGylated lipids (e.g., C14-PEG2k, C18-PEG2k, C18-PEG5K, DMPE-PEG2k, etc.). In some embodiments, an NLP composition comprises a molar ratio of at least 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% of one or more PEGylated lipids. In some embodiments, an NLP composition comprises about 0.1%-10% (w/w) PEGylated lipid (e.g., C14-PEG2k, C18-PEG2k, DMPE-PEG2k, etc.). In some embodiments, an NLP composition comprises about 1%-3% of one or more PEGylated lipids. In some embodiments, an NLP composition comprises about 1.5% of one or more PEGylated lipids. In some embodiments, an NLP composition comprises about 2.5% of one or more PEGylated lipids.

In some embodiments, about 5%-50% (w/w) of the lipids in an NLP composition is PEGylated lipid (e.g., about 10%-20% of the lipids in an NLP composition is PEGylated lipid, e.g., about 10%, 12.5%, 16%, or 20% of the lipids in an NLP composition is PEGylated lipid). In some embodiments, about 30%-75% (e.g., about 35% or about 50% PEGylated lipid) of the lipids in an NLP composition is PEGylated lipid. In some embodiments, about 35%-50% (e.g., about 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%) of the lipids in an NLP composition is PEGylated lipid.

In some embodiments, an NLP composition comprising one or more PEGylated lipids has enhanced uptake relative to an NLP composition not comprising the one or more PEGylated lipids. In some embodiments, an NLP composition comprising one or more PEGylated lipids has altered stability (e.g., increased stability, decreased stability, etc.) relative to an NLP composition that not comprising the one or more PEGylated lipids. In some embodiments, an NLP composition comprising one or more PEGylated lipids has altered particle size relative to an NLP composition not comprising the one or more PEGylated lipids. In some embodiments, an NLP composition comprising one or more PEGylated lipids is less likely to be phagocytosed by a cell than an NLP composition not comprising the one or more PEGylated lipids. In some embodiments, an NLP composition comprises one or more surface modifiers comprising one or more PEG moieties having a molecular weight of the head group from about 750 Da to about 20,000 Da. In some embodiments, an NLP composition comprises one or more surface modifiers comprising one or more PEG moieties having a molecular weight of the head group from about 750 Da to about 12,000 Da. In some embodiments, an NLP composition comprises one or more surface modifiers comprising one or more PEG moieties having a molecular weight of the head group between about 1,000 Da to about 5,000 Da. In some embodiments, an NLP composition comprises one or more neutral (uncharged) lipopolymers. In some embodiments, an NLP composition comprises one or more positively charged lipopolymers. In some embodiments, an NLP composition comprises one or more negatively charged lipopolymers. In some embodiments, an NLP composition comprises one or more neutral distearoyl glycerol and the negatively charged distearoyl phosphatidylethanolamine, both covalently attached to methoxy poly(ethylene glycol) (mPEG or PEG) of Mw 750, 2000, 5000, or 12000.

### C. Cargo

In some embodiments, the NLPs comprise a hydrophilic functional agent. The hydrophilic functional agent may be any water-soluble or polar compound that exerts a biological effect on a plant, plant pest, or symbiont. Non-limiting examples include: Small molecule agrochemicals, such as hydrophilic herbicides (e.g., glufosinate, phosphonic acid), antibiotics (e.g., streptomycin, oxytetracycline), and antifungals (e.g., fosetyl-Al); Phytohormones and growth regulators, including salicylic acid, jasmonic acid, abscisic acid, cytokinins, and auxins, particularly in their salt or conjugated forms; Nutrients or micronutrients, including chelated minerals (e.g., Zn-EDTA), amino acids (e.g., proline), or water-soluble vitamins (e.g., thiamine); Immunity elicitors and plant defense activators, such as oligogalacturonides, chitosan derivatives, and β-glucans; Hydrophilic dyes or reporters, such as sulforhodamine B or fluorescein, useful for tracking or confirming NLP uptake and distribution in plant tissues.

In some embodiments, the hydrophilic functional agent is encapsulated in the hydrophilic core of the NLP. In other embodiments, the agent is associated with the surface of the NLP or is co-delivered in an aqueous phase. Hydrophilic cargo may be used alone or in combination with nucleic acids or peptides to enhance delivery, targeting, or functional outcomes. The encapsulation of hydrophilic agents in NLPs enhances their stability, biodistribution, and uptake in plant tissues, and may reduce environmental degradation or off-target effects.

In some embodiments, the NLPs comprise a polynucleotide, e.g. an mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), an ADAR targeting oligonucleotide, an antisense oligonucleotide, a long non-coding RNA, a ceDNA, a minicircle, a miniplasmid, a viroid, a virus, or a DNA molecule encoding any of these RNAs.

In some embodiments, the polynucleotide cargo is encapsulated within the hydrophilic core of the NLP. Encapsulation in the aqueous interior improves solubility and stability of the polynucleotide and allows for efficient delivery across plant cell walls and membranes. The use of charged or ionizable lipids promotes complexation and retention of the polynucleotide in the aqueous interior.

### a) Heterologous Polynucleotide

In some instances, the NLPs described herein comprises a heterologous nucleic acid (heterologous polynucleotide). Numerous nucleic acids are useful in the rNLP compositions and methods described herein. The NLPs disclosed herein may include any number or type (e.g., classes) of heterologous nucleic acids (e.g., DNA molecule (e.g., plasmid) or RNA molecule, e.g., mRNA, guide RNA (gRNA), or inhibitory RNA molecule or precursor thereof(e.g., siRNA, shRNA, or miRNA or a precursor of any of these), or a hybrid DNA-RNA molecule), such as at least about 1 class or variant of a nucleic acid, or 2, 3, 4, 5, 10, 15, 20, or more classes or variants of nucleic acids. A suitable concentration of each nucleic acid in the composition depends on factors such as efficacy, stability of the nucleic acid, number of distinct nucleic acids, the formulation, and methods of application of the composition. Examples of nucleic acids useful herein include a DNA molecule (e.g., a plasmid), an mRNA, an siRNA, a Dicer substrate small interfering RNA (dsiRNA), an antisense oligonucleotide, an antisense RNA, a short interfering RNA (siRNA) or siRNA precursor (e.g., one or more strands of RNA that hybridize inter- or intra-molecularly to form at least partially double-stranded RNA having at least about 20 contiguous base-pairs), a short hairpin (shRNA), a microRNA (miRNA) or miRNA precursor, an asymmetric interfering RNA (aiRNA), a peptide nucleic acid (PNA), a morpholino, a locked nucleic acid (LNA), a piwi-interacting RNA (piRNA), a ribozyme, a deoxyribozymes (DNAzyme), an aptamer (DNA, RNA), a circular RNA (circRNA), a guide RNA (gRNA), an ADAR (adenosine deaminases that act on RNA) targeting oligonucleotide, a long non-coding RNA, a closed-ended DNA (ceDNA), a minicircle, a miniplasmid, or a DNA molecule encoding any of the recited RNAs.

A NLP composition including a nucleic acid as described herein can be contacted with a plant in an amount and for a time sufficient to: (a) reach a target level (e.g., a predetermined or threshold level) of nucleic acid concentration; and (b) modify the plant (e.g., increase the fitness of the plant).

### (i) Nucleic Acids Encoding Polypeptides

In some instances, the NLPs include a heterologous nucleic acid encoding a polypeptide.

Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The NLP composition may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include non-transcribed elements, such as an origin of replication, a suitable promoter and enhancer, and other 5′ or 3′ flanking non-transcribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instances, the invention includes a composition to alter expression of one or more proteins, e.g., proteins that affect activity, structure, or function. Expression of the one or more proteins may be restricted to a specific location(s) or widespread throughout the organism.

An NLP as described herein comprising an mRNA can be used to measure NLP uptake and mRNA expression by a plant. Following uptake of the NLPs, expression of mRNA's encoding for CmFT, TFL1, JMJ17 affect plant flowering. Further, mRNA's encoding for PDS3, RIF10, SPC1, APG10 or PORB/PORB affects plant leaf color and causes whitening.

(ii) Synthetic mRNA

The NLP composition may include a synthetic mRNA molecule, e.g., a synthetic mRNA molecule encoding a polypeptide. The synthetic mRNA molecule can be modified, e.g., chemically. The mRNA molecule can be chemically synthesized or transcribed in vitro. The mRNA molecule can be encoded on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, Capl, ARCA, inosine, Nl-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′UTR. Such modifications are known and are described, e.g., in WO 2012/135805 and WO 2013/052523. Additional terminal modifications are described, e.g., in WO 2014/164253 and WO 2016/011306, WO 2012/045075, and WO 2014/093924. Chimeric enzymes for synthesizing capped RNA molecules (e.g., modified mRNA) which may include at least one chemical modification are described in WO 2014/028429.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid) (PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667. Any of the foregoing may be synthesized as an IVT polynucleotide, chimeric polynucleotide or a circular polynucleotide, and each may include one or more modified nucleotides or terminal modifications.

(iii) Inhibitory RNA

In some instances, the NLP composition includes an inhibitory RNA molecule, e.g., that acts via the RNA interference (RNAi) pathway. In some instances, the inhibitory RNA molecule decreases the level of gene expression in a plant and/or decreases the level of a protein in the plant. In some instances, the inhibitory RNA molecule inhibits expression of a plant gene. For example, an inhibitory RNA molecule may include a short interfering RNA or its precursor, short hairpin RNA, and/or a microRNA or its precursor that targets a gene in the plant. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to: short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276). A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 21 or 22 nucleotides. MiRNAs bind to target sites on mRNA molecules and silence the mRNA, e.g., by causing cleavage of the mRNA, destabilization of the mRNA, or inhibition of translation of the mRNA. In some instances, the inhibitory RNA molecule decreases the level and/or activity of a negative regulator of function. In other instances, the inhibitor RNA molecule decreases the level and/or activity of an inhibitor of a positive regulator of function. The inhibitory RNA molecule can be chemically synthesized or transcribed in vitro.

In some instances, the nucleic acid is a DNA, a RNA, or a PNA. In some instances, the RNA is an inhibitory RNA. In some instances, the inhibitory RNA inhibits gene expression in a plant. In some instances, the nucleic acid is an mRNA, a modified mRNA, or a DNA molecule that, in the plant, increases expression of an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone. In some instances, the nucleic acid is an mRNA, a modified mRNA, or a DNA molecule that increases the expression of an enzyme (e.g., a metabolic enzyme, a recombinase enzyme, a helicase enzyme, an integrase enzyme, a RNAse enzyme, a DNAse enzyme, or an ubiquitination protein), a pore-forming protein, a signaling ligand, a cell penetrating peptide, a transcription factor, a receptor, an antibody, a nanobody, a gene editing protein (e.g., a CRISPR-Cas system, a TALEN, or a zinc finger), a riboprotein, a protein aptamer, or a chaperone. In some aspects, the nucleic acid encodes the enzyme, pore-forming protein, signaling ligand, cell penetrating peptide, transcription factor, receptor, antibody, nanobody, gene editing protein, riboprotein, protein aptamer, or chaperone. In some instances, the increase in expression in the plant is an increase in expression of about 5%, 10%, 15%, 20%, 30%, 40%, 50%, 60%, 70%, 80%, 90%, 100%, or more than 100% relative to a reference level (e.g., the expression in an untreated plant). In some instances, the increase in expression in the plant is an increase in expression of about 2× fold, about 4× fold, about 5× fold, about 1Ox fold, about 20× fold, about 25× fold, about 50× fold, about 75× fold, or about 100× fold or more, relative to a reference level (e.g., the expression in an untreated plant).

In some instances, the nucleic acid is an antisense RNA, a dsiRNA, a siRNA, a shRNA, a miRNA, an aiRNA, a PNA, a morpholino, a LNA, a piRNA, a ribozyme, a DNAzyme, an aptamer (DNA, RNA), a circRNA, a gRNA, or a DNA molecules (e.g., a plasmid) that acts to reduce, in the plant, expression of, e.g., an enzyme (a metabolic enzyme, a recombinase enzyme, a helicase enzyme, an integrase enzyme, a RNAse enzyme, a DNAse enzyme, a polymerase enzyme, a ubiquitination protein, a superoxide management enzyme, or an energy production enzyme), a transcription factor, a secretory protein, a structural factor (actin, kinesin, or tubulin), a riboprotein, a protein aptamer, a chaperone, a receptor, a signaling ligand, or a transporter. In some instances, the decrease in expression in the plant is a decrease in expression of about 5%, 10%, 15%, 20%, 30%, 40%, 50%, 60%, 70%, 80%, 90%, 100%, or more than 100% relative to a reference level (e.g., the expression in an untreated plant). In some instances, the decrease in expression in the plant is a decrease in expression of about 2× fold, about 4× fold, about 5× fold, about 1Ox fold, about 20× fold, about 25× fold, about 50× fold, about 75× fold, or about 100× fold or more, relative to a reference level (e.g., the expression in an untreated plant).

RNAi molecules include a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for a target gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided as ready-to-use RNA synthesized in vitro or as sense and antisense RNA sequences (or DNA encoding sense and antisense RNA sequences) transfected into cells which will yield RNAi molecules upon transcription. Hybridization of the RNA molecule with, e.g., the target mRNA results in degradation of the hybridized complex by RNAse H and/or inhibition of the formation of translation complexes. Both result in a failure to produce the product of the original gene.

The length of the RNAi molecule that hybridizes to the transcript of interest may be around 10 nucleotides, between about 15 or 30 nucleotides, or about 15, 16, 17, 18, 19, 20, 21, 22, 23, 24, 25, 26, 27, 28, 29, 30 or more nucleotides. In embodiments, the RNAi molecule hybridizes to the transcript of interest to form a perfectly or near-perfectly double-stranded region of at least about 17 base pairs; in embodiments the double-stranded region includes at least about 10 contiguous base pairs. The degree of identity of the antisense sequence to the targeted transcript may be at least 75%, at least 80%, at least 85%, at least 90%, or at least 95.

RNAi molecules may also include overhangs. Typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In some instances, both the sense strand and the antisense strand contain 3′ and 5′ overhangs. In some instances, one or more of the 3′ overhang nucleotides of one strand base pairs with one or more 5′ overhang nucleotides of the other strand. In other instances, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another instance, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules include a nucleotide sequence that is identical to about 15 to about 25 contiguous nucleotides of the target mRNA. In some instances, the siRNA sequence commences with the dinucleotide AA, includes a GC-content of about 30-70% (about 30-60%, about 40-60%, or about 45%-55%), and does not have a high percentage identity to any nucleotide sequence other than the target in the genome in which it is to be introduced, for example as determined by standard BLAST search. siRNAs and shRNAs resemble intermediates in the processing pathway of the endogenous microRNA (miRNA) genes (Bartel, Cell 116:281-297, 2004). In some instances, siRNAs can function as miRNAs and vice versa (Zeng et al., Mol. Cell 9:1327-1333, 2002; Doench et al., Genes Dev. 17:438-442, 2003). Exogenous siRNAs downregulate mRNAs with seed complementarity to the siRNA (Birmingham et al., Nat. Methods 3:199-204, 2006). Multiple target sites within a 3′ UTR give stronger downregulation (Doench et al., Genes Dev. 17:438-442, 2003).

Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al., Nat. Methods 3(9):670-676, 2006; Reynolds et al., Nat. Biotechnol. 22(3):326-330, 2004; Khvorova et al., Nat. Struct. Biol. 10(9):708-712, 2003; Schwarz et al., Cell 115(2):199-208, 2003; Ui-Tei et al., Nucleic Acids Res. 32(3):936-948, 2004; Heale et al., Nucleic Acids Res. 33(3):e30, 2005; Chalk et al., Biochem. Biophys. Res. Commun. 319(1):264-274, 2004; and Amarzguioui et al., Biochem. Biophys. Res. Commun. 316(4):1050-1058, 2004).

The RNAi molecule modulates expression of RNA encoded by a gene. Because multiple genes can share some degree of sequence homology with each other, in some instances, the RNAi molecule can be designed to target a class of genes with sufficient sequence homology. In some instances, the RNAi molecule can contain a sequence that has complementarity to sequences that are shared amongst different gene targets or are unique for a specific gene target. In some instances, the RNAi molecule can be designed to target conserved regions of an RNA sequence having homology between several genes thereby targeting several genes in a gene family (e.g., different gene isoforms, splice variants, mutant genes, etc.). In some instances, the RNAi molecule can be designed to target a sequence that is unique to a specific RNA sequence of a single gene.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that such modifications can increase nuclease resistance and/or serum stability, or decrease immunogenicity.

In some instances, the RNAi molecule or its precursor is linked to a delivery polymer via a physiologically labile bond or linker. The physiologically labile linker is selected such that it undergoes a chemical transformation (e.g., cleavage) when present in certain physiological conditions, (e.g., disulfide bond cleaved in the reducing environment of the cell cytoplasm). Release of the molecule from the polymer, by cleavage of the physiologically labile linkage, facilitates interaction of the molecule with the appropriate cellular components for activity.

The RNAi molecule-polymer conjugate may be formed by covalently linking the molecule to the polymer. The polymer is polymerized or modified such that it contains a reactive group A. The RNAi molecule is also polymerized or modified such that it contains a reactive group B. Reactive groups A and B are chosen such that they can be linked via a reversible covalent linkage using methods known in the art.

Conjugation of the RNAi molecule to the polymer can be performed in the presence of an excess of polymer. Because the RNAi molecule and the polymer may be of opposite charge during conjugation, the presence of excess polymer can reduce or eliminate aggregation of the conjugate. Alternatively, an excess of a carrier polymer, such as a polycation, can be used. The excess polymer can be removed from the conjugated polymer prior to administration of the conjugate. Alternatively, the excess polymer can be co-administered with the conjugate.

The making and use of inhibitory agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press (2010).

NLP comprising a siRNA can be used to measure NLP uptake and interference of RNA expression by a plant. Following uptake of the NLPs, expression of siRNA's targeting miR156 affect plant flowering, and expression of siRNA's targeting GUN4, JMJ17, TRY/CPC/ETC2 and PDS3 affect plant leaf color and cause whitening. Further, expression of siRNA's targeting EEP1 cause extra petals, and targeting PP2A cause a dwarf and agravitropic phenotype.

### (iv) Gene Editing Elements

The NLP compositions described herein may include a component of a gene editing system. For example, the agent may introduce an alteration (e.g., insertion, deletion (e.g., knockout), translocation, inversion, single point mutation, or other mutation) in a gene in the plant. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31 (7):397-405, 2013.

In a typical CRISPR/Cas system, an endonuclease is directed to a target nucleotide sequence (e.g., a site in the genome that is to be sequence-edited) by sequence-specific, non-coding guide RNAs that target single- or double-stranded DNA sequences. Three classes (1-11l) of CRISPR systems have been identified. The class II CRISPR systems use a single Cas endonuclease (rather than multiple Cas proteins). One class II CRISPR system includes a type II Cas endonuclease such as Cas9, a CRISPR RNA (crRNA), and a trans-activating crRNA (tracrRNA). The crRNA contains a guide RNA, e.g., typically an about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The RNAs serve as guides to direct Cas proteins to silence specific DNA/RNA sequences, depending on the spacer sequence. See, e.g., Horvath et al., Science 327:167-170, 2010; Makarova et al., Biology Direct 1:7, 2006; Pennisi, Science 341:833-836, 2013. The target DNA sequence must generally be adjacent to a protospacer adjacent motif (PAM) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningitidis).

Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words a Cpf1 system requires only the Cpf1 nuclease and a crRNA to cleave the target DNA sequence. Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al., Cell 163:759-771, 2015.

For the purposes of gene editing, CRISPR arrays can be designed to contain one or multiple guide RNA sequences corresponding to a desired target DNA sequence; see, for example, Cong et al., Science 339:819-823, 2013; Ran et al., Nature Protocols 8:2281-2308, 2013. At least about 16 or 17 nucleotides of gRNA sequence are required by Cas9 for DNA cleavage to occur; for Cpf1 at least about 16 nucleotides of gRNA sequence is needed to achieve detectable DNA cleavage. In practice, guide RNA sequences are generally designed to have a length of between 17-24 nucleotides (e.g., 19, 20, or 21 nucleotides) and complementarity to the targeted gene or nucleic acid sequence. Custom gRNA generators and algorithms are available commercially for use in the design of effective guide RNAs.

Gene editing has also been achieved using a chimeric single guide RNA (sgRNA), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al., Nature Biotechnol. 985-991, 2015.

Whereas wild-type Cas9 generates double-strand breaks (DSBs) at specific DNA sequences targeted by a gRNA, a number of CRISPR endonucleases having modified functionalities are available, for example: a nickase version of Cas9 generates only a single-strand break; a catalytically inactive Cas9 (dCas9) does not cut the target DNA but interferes with transcription by steric hindrance. dCas9 can further be fused with an effector to repress (CRISPRi) or activate (CRISPRa) expression of a target gene. For example, Cas9 can be fused to a transcriptional repressor (e.g., a KRAB domain) or a transcriptional activator (e.g., a dCas9-VP64 fusion). A catalytically inactive Cas9 (dCas9) fused to Fokl nuclease (dCas9-Fokl) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A double nickase Cas9 that introduces two separate double-strand breaks, each directed by a separate guide RNA, is described as achieving more accurate genome editing by Ran et al., Cell 154:1380-1389, 2013.

CRISPR technology for editing the genes of eukaryotes is disclosed in US Patent Application Publications US 2016/0138008 A1 and US 2015/0344912 A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some instances, the desired genome modification involves homologous recombination, wherein one or more double-stranded DNA breaks in the target nucleotide sequence is generated by the RNA-guided nuclease and guide RNA(s), followed by repair of the break(s) using a homologous recombination mechanism (homology-directed repair). In such instances, a donor template that encodes the desired nucleotide sequence to be inserted or knocked-in at the double-stranded break is provided to the cell or subject; examples of suitable templates include single-stranded DNA templates and double-stranded DNA templates (e.g., linked to the polypeptide described herein). In general, a donor template encoding a nucleotide change over a region of less than about 50 nucleotides is provided in the form of single-stranded DNA; larger donor templates (e.g., more than 100 nucleotides) are often provided as double-stranded DNA plasmids. In some instances, the donor template is provided to the cell or subject in a quantity that is sufficient to achieve the desired homology-directed repair but that does not persist in the cell or subject after a given period of time (e.g., after one or more cell division cycles). In some instances, a donor template has a core nucleotide sequence that differs from the target nucleotide sequence (e.g., a homologous endogenous genomic region) by at least 1, at least 5, at least 10, at least 20, at least 30, at least 40, at least 50, or more nucleotides. This core sequence is flanked by homology arms or regions of high sequence identity with the targeted nucleotide sequence; in some instances, the regions of high identity include at least 10, at least 50, at least 100, at least 150, at least 200, at least 300, at least 400, at least 500, at least 600, at least 750, or at least 1000 nucleotides on each side of the core sequence. In some instances where the donor template is in the form of a single-stranded DNA, the core sequence is flanked by homology arms including at least 10, at least 20, at least 30, at least 40, at least 50, at least 60, at least 70, at least 80, or at least 100 nucleotides on each side of the core sequence. In instances, where the donor template is in the form of a double-stranded DNA, the core sequence is flanked by homology arms including at least 500, at least 600, at least 700, at least 800, at least 900, or at least 1000 nucleotides on each side of the core sequence. In one instance, two separate double strand breaks are introduced into the cell or subject's target nucleotide sequence with a double nickase Cas9 (see Ran et al., Cell 154:1380-1389, 2013), followed by delivery of the donor template.

In some instances, the composition includes a gRNA and a targeted nuclease, e.g., a Cas9, e.g., a wild type Cas9, a nickase Cas9 (e.g., Cas9 D10A), a dead Cas9 (dCas9), eSpCas9, Cpf 1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (sgRNA) to modulate activity and/or expression of one or more target nucleic acids sequences.

In instances, the agent includes a guide RNA (gRNA) for use in a CRISPR system for gene editing. In some instances, the agent includes a zinc finger nuclease (ZFN), or a mRNA encoding a ZFN, that targets (e.g., cleaves) a nucleic acid sequence (e.g., DNA sequence) of a gene in the plant. In some instances, the agent includes a TALEN, or an mRNA encoding a TALEN, that targets (e.g., cleaves) a nucleic acid sequence (e.g., DNA sequence) in a gene in the plant.

For example, the gRNA can be used in a CRISPR system to engineer an alteration in a gene in the plant. In other examples, the ZFN and/or TALEN can be used to engineer an alteration in a gene in the plant. Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration increases the level and/or activity of a gene in the plant. In other examples, the alteration decreases the level and/or activity of (e.g., knocks down or knocks out) a gene in the plant. In yet another example, the alteration corrects a defect (e.g., a mutation causing a defect) in a gene in the plant.

In some instances, the CRISPR system is used to edit (e.g., to add or delete a base pair) a target gene in the plant. In other instances, the CRISPR system is used to introduce a premature stop codon, e.g., thereby decreasing the expression of a target gene. In yet other instances, the CRISPR system is used to turn off a target gene in a reversible manner, e.g., similarly to RNA interference. In some instances, the CRISPR system is used to direct Cas to a promoter of a gene, thereby blocking an RNA polymerase sterically.

In some instances, a CRISPR system can be generated to edit a gene in the plant, using technology described in, e.g., U.S. Publication No. 20140068797, Cong, Science 339: 819-823, 2013; Tsai, Nature Biotechnol. 32:6 569-576, 2014; U.S. Pat. No. 8,871,445; 8,865,406; 8,795,965; 8,771,945; and 8,697,359.

In some instances, the CRISPR interference (CRISPRi) technique can be used for transcriptional repression of specific genes in the plant. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some instances, CRISPR-mediated gene activation (CRISPRa) can be used for transcriptional activation of a gene in the plant. In the CRISPRa technique, dCas9 fusion proteins recruit transcriptional activators. For example, dCas9 can be fused to polypeptides (e.g., activation domains) such as VP64 or the p65 activation domain (p65D) and used with sgRNA (e.g., a single sgRNA or multiple sgRNAs), to activate a gene or genes in the plant. Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5-15, 2016, incorporated herein by reference. In addition, dCas9-mediated epigenetic modifications and simultaneous activation and repression using CRISPR systems, as described in Dominguez et al., can be used to modulate a gene in the plant.

In some embodiments, functional delivery of short nucleic acids in planta affects gene expression in a plant. In some embodiments, the plant is a transgenic dCas9-SunTag-VP64 Arabidopsis thaliana plant as described by Papikian et al., Nat Commun. 13; 10(1):729 (2019). In this system, a deactivated Cas9 endonuclease (dCas9) is fused to GCN4 peptide repeats, and the transcriptional activator VP64 is fused to a single chain variable fragment GCN4 antibody, allowing multiple copies of VP64 to associate with a single dCas9 protein. By supplying gRNAs corresponding to the promoter sequence of a gene of interest (e.g. the Flowering Wageningen gene, or FWA), this dCas9-SunTag-VP64 system can be used as a homing device' to activate gene expression.

### E. Polypeptide Cargo

The NLP composition (e.g., pNLPs) described herein may comprise a heterologous polypeptide. In some instances, the NLP composition described herein includes a polypeptide or functional fragments or derivative thereof that modifies a plant (e.g., e.g., increases the fitness of the plant). For example, the polypeptide can increase the fitness of a plant. A NLP composition including a polypeptide as described herein can be contacted with a plant in an amount and for a time sufficient to: (a) reach a target level (e.g., a predetermined or threshold level) of polypeptide concentration; and (b) modify the plant (e.g., increase the fitness of the plant).

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptides described herein may be formulated in a composition for any of the uses described herein. The compositions disclosed herein may include any number or type (e.g., classes) of polypeptides, such as at least about any one of 1 polypeptide, 2, 3, 4, 5, 10, 15, 20, or more polypeptides. A suitable concentration of each polypeptide in the composition depends on factors such as efficacy, stability of the polypeptide, number of distinct polypeptides in the composition, the formulation, and methods of application of the composition. In some instances, each polypeptide in a liquid composition is from about 0.1 ng/mL to about 100 mg/mL. In some instances, each polypeptide in a solid composition is from about 0.1 ng/g to about 100 mg/g.

Methods of making a polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologies Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some instances, the NLP composition includes an antibody or antigen binding fragment thereof. For example, an agent described herein may be an antibody that blocks or potentiates activity and/or function of a component of the plant. The antibody may act as an antagonist or agonist of a polypeptide (e.g., enzyme or cell receptor) in the plant. The making and use of antibodies against a target antigen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### F. Heterologous Agricultural Agents

Several embodiments relate to an NLP composition further comprising one or more heterologous functional agents, such as one or more of a pesticidal agent, fertilizing agent, herbicidal agent, plant-modifying agent, antifungal agent, an anti-oomycete agent, an antibacterial agent, a virucidal agent, an anti-viral agent, an insecticidal agent, a nematocidal agent, an antiparasitic agent, and an insect repellent. In some embodiments of an NLP composition as described herein, an NLP may encapsulate the heterologous functional agent. In some embodiments of an NLP composition as described herein, the heterologous functional agent can be embedded on or conjugated to the surface of the NLP. In some embodiments, an NLP composition may include two or more (e.g., 2, 3, 4, 5, 6, 7, 8, 9, 10, or more than 10) different heterologous functional agents. Heterologous functional agents may be added at any step during the manufacturing process effective to introduce the agent into the NLP composition. In some embodiments, the heterologous functional agent is unencapsulated.

In some embodiments, one or more heterologous functional agents comprised in an NLP composition may be any of the pesticidal agents disclosed herein. In some embodiments, a pesticidal agent may be a naturally occurring or synthetic insecticide (e.g., a larvicide, an adulticide, etc.). In some embodiments, a pesticidal agent may be a naturally occurring or synthetic insect growth regulator. In some embodiments, a pesticidal agent may be a naturally occurring or synthetic acaricide (miticides). In some embodiments, a pesticidal agent may be a naturally occurring or synthetic molluscicide, nematicide, ectoparasiticide, bactericide, fungicide, or herbicide. The term “pesticidal agent” may further encompass other bioactive molecules such as antibiotics, antivirals pesticides, antifungals, antihelminthics, nutrients, and/or agents that stun or slow insect movement, fecundity, etc. In some embodiments, a heterologous functional agent may be a therapeutic agent (e.g., a cell-penetrating agent, an antifungal agent, an antibacterial agent, a virucidal agent, an anti-viral agent, an insecticidal agent, a nematicidal agent, an antiparasitic agent, an insect repellent, etc.). In some embodiments, an NLP composition includes two or more (e.g., 2, 3, 4, 5, 6, 7, 8, 9, 10, or more than 10) different heterologous functional agents, e.g. deltamethrin and permethrin. In some embodiments, an NLP composition as described herein may comprise one or more heterologous functional agents described in PCT/US2024/015064, which is incorporated in its entirety. Exemplary pesticidal agents are disclosed in PCT/US2024/015064, which is incorporated in its entirety herein.

Several embodiments relate to an NLP composition as described herein comprising one or more insecticidal agents. In some instances, an NLP composition includes two or more (e.g., 2, 3, 4, 5, 6, 7, 8, 9, 10, or more than 10) different insecticidal agents. Several embodiments relate to a method of decreasing the fitness (e.g., decrease growth or kill) of a targeted insect by contacting the targeted insect or a plant or animal infested with or parasitized by the targeted insect with an NLP composition including an insecticidal agent, in an amount and for a time sufficient to: (a) reach a target level (e.g., a predetermined or threshold level) of insecticidal agent concentration inside or on the target insect; and (b) decrease fitness of the target insect. Insecticidal agents may be loaded/incorporated into an NLP composition as described herein and/or formulated with an NLP composition by any of the methods described herein.

In some embodiments, a heterologous functional agent (e.g., a heterologous agricultural agent (e.g., pesticidal agent, fertilizing agent, herbicidal agent, plant-modifying agent, a heterologous nucleic acid, a heterologous polypeptide, a heterologous small molecule, etc.) or a heterologous therapeutic agent (e.g., an antifungal agent, an anti-oomycete agent, an antibacterial agent, a virucidal agent, an anti-viral agent, a nematicidal agent, an antiparasitic agent, an insect repellent, etc.)) can be modified. For example, the modification can be a chemical modification, e.g., conjugation to a marker, e.g., fluorescent marker or a radioactive marker. In some embodiments, the modification can include conjugation or operational linkage of a heterologous functional agent to a moiety that enhances the stability, delivery, targeting, bioavailability, or half-life of the agent, e.g., a lipid, a glycan, a polymer (e.g., PEG), or a cation moiety.

In some embodiments herein, methods are provided for encapsulating bioactives with a high volatility, e.g. having a high vapor pressure. Exemplary volatile agents used in the field of agriculture include but are not limited to herbicides (e.g. Dicamba or 2,4-D), fumigants, pheromones, and essential oils.

### G. Agricultural Formulations

To allow ease of application, handling, transportation, storage, and effective activity, NLPs, can be formulated with other substances. NLPs can be formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph no 2, 5th Edition by CropLife International (2002).

NLP compositions can be applied as aqueous suspensions or emulsions prepared from concentrated formulations of such agents. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the rNLP composition, a carrier, and surfactants. The carrier is usually selected from among the attapulgite clays, the montmorillonite clays, the diatomaceous earths, or the purified silicates. Effective surfactants, including from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of NLPs, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble NLP compositions dispersed in an aqueous carrier at a concentration in the range from about 5% to about 50% by weight. Suspensions are prepared by finely grinding the composition and vigorously mixing it into a carrier comprised of water and surfactants. Ingredients, such as inorganic salts and synthetic or natural gums may also be added, to increase the density and viscosity of the aqueous carrier.

NLP compositions may also be applied as granular compositions that are particularly useful for applications to the soil. Granular compositions usually contain from about 0.5% to about 10% by weight of the NLP composition, dispersed in a carrier that comprises clay or a similar substance. Such compositions are usually prepared by dissolving the formulation in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing the present NLP formulation are prepared by intimately mixing NLPs in powdered form with a suitable dusty agricultural carrier, such as kaolin clay, ground volcanic rock, and the like. Dusts can suitably contain from about 1% to about 10% of the packets. They can be applied as a seed dressing or as a foliage application with a dust blower machine.

It is equally practical to apply the present formulation in the form of a solution in an appropriate organic solvent, usually petroleum oil, such as the spray oils, which are widely used in agricultural chemistry.

NLPs can also be applied in the form of an aerosol composition. In such compositions the packets are dissolved or dispersed in a carrier, which is a pressure-generating propellant mixture. The aerosol composition is packaged in a container from which the mixture is dispensed through an atomizing valve.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer including: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007. For ease of use, this embodiment will be referred to as “OIWE.”

Additionally, generally, when the molecules disclosed above are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO (ethylene oxide-propylene oxide) block copolymers; and graft copolymers. An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the NLP composition on the target. The types of surfactants used for bioenhancement depend generally on the nature and mode of action of the NLP composition. However, they are often non-ionics such as: alkyl ethoxylates; linear aliphatic alcohol ethoxylates; aliphatic amine ethoxylates.

A carrier or diluent in an agricultural formulation is a material added to the NLP composition to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of NLP composition when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol, and polyethylene oxide. Another good anti settling agent is xanthan gum.

Microorganisms can cause spoilage of formulated products. Therefore, preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BT.

The presence of surfactants often causes water-based formulations to foam during mixing operations in production and in application through a spray tank. In order to reduce the tendency to foam, anti-foam agents are often added either during the production stage or before filling into bottles.

Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g., plant and animal sources. Specific examples are: vegetable oils, seed oils, and esters thereof, also alkoxylated alkyl polyglucosides.

In some instances, NLPs can be freeze-dried or lyophilized. See U.S. Pat. No. 4,311,712. The rNLPs can later be reconstituted on contact with water or another liquid. Other components can be added to the lyophilized or reconstituted NLPs, for example, other heterologous functional agents, agriculturally acceptable carriers, or other materials in accordance with the formulations described herein.

Other optional features of the composition include carriers or delivery vehicles that protect the NLP composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

For further information on agricultural formulations, see “Chemistry and Technology of Agrochemical Formulations” edited by D. A. Knowles, copyright 1998 by Kluwer Academic Publishers. Also see “Insecticides in Agriculture and Environment—Retrospects and Prospects” by A. S. Perry, I. Yamamoto, I. Ishaaya, and R. Perry, copyright 1998 by Springer-Verlag.

### Pharmaceutical Formulations

The modified NLPs described herein can be formulated into pharmaceutical compositions, e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed. A NLP composition may be formulated for e.g., oral administration, intravenous administration (e.g., injection or infusion), or subcutaneous administration to an animal. For injectable formulations, various effective pharmaceutical carriers are known in the art (See, e.g., Remington: The Science and Practice of Pharmacy, 22nd ed., (2012) and ASFIP Handbook on Injectable Drugs, 18th ed., (2014)).

Pharmaceutically acceptable carriers and excipients in the present compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol.

The compositions may be formulated according to conventional pharmaceutical practice. The concentration of the compound in the formulation will vary depending upon a number of factors, including the dosage of the active agent (e.g., NLP) to be administered, and the route of administration.

### H. Mixtures of NLPs

In some embodiments, the compositions described herein comprise a mixture of two or more different NLP populations, each differing in one or more physicochemical or functional properties. Such mixtures may include NLPs of different size, surface charge, core polarity, lipid composition, or encapsulated cargo. The use of heterogeneous NLP formulations may allow for multiplexed delivery of functional agents, improved tissue distribution, enhanced stability, or synergistic biological effects in a plant.

In some embodiments, the composition comprises a first population of NLPs comprising a hydrophilic core and a second population comprising a hydrophobic core. The hydrophilic-core NLPs may encapsulate polar or water-soluble agents such as RNA, peptides, or plant hormones, while the hydrophobic-core NLPs may encapsulate lipophilic pesticides, essential oils, or fatty acid derivatives. Each NLP population may be independently formulated, stabilized, or surface-modified, and then combined into a single composition for delivery.

In some embodiments, each NLP population may be optimized for delivery to a different plant compartment. For example, hydrophilic-core NLPs may be optimized for translocation into the vascular system or uptake into meristematic tissue, while hydrophobic-core NLPs may adhere to or penetrate cuticular layers or pest surfaces. In certain embodiments, a mixed NLP composition allows for simultaneous delivery of synergistic cargo, such as an siRNA targeting gene expression and a pesticide affecting metabolic function.

In some embodiments, the composition comprises NLPs of different zeta potentials or sizes. For example, a combination of NLPs with neutral and positively charged surfaces may enhance uptake in different plant tissues or balance systemicity with retention. In some embodiments, a mixture of NLPs with different PEGylation levels or surface modifiers (e.g., pectin derivatives, boronic acid lipids) is used to fine-tune distribution and delivery kinetics.

In some embodiments, NLPs comprising different functional agents are prepared separately and combined post-manufacture, while in other embodiments, they are co-formulated using multi-stream microfluidics or sequential emulsification. The ratio of hydrophilic- to hydrophobic-core NLPs may range from 1:99 to 99:1, depending on the delivery objectives, cargo compatibility, and plant species targeted.

### I. Kits

The present invention also provides a kit including e.g. a container having an NLP composition described herein. The kit may further include instructional material for applying or delivering the NLP composition to a plant in accordance with a method of the present invention. The skilled artisan will appreciate that the instructions for applying the NLP composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### II. Methods

### A. Production Methods

In some embodiments, an organic phase comprising one or more of phospholipids, sterols, synthetic lipids, and optionally pegylated lipids, pectin derivatives and boronic acid lipids, is mixed with an aqueous phase comprising de-ionized water and a hydrophilic heterologous functional agent, e.g. a polynucleotide, applying a source of energy (e.g. heat, pressure) to facilitate the formation of NLPs. In some embodiments, NLPs are produced with a hand-pipetting method by mixing a lipid solution (ethanolic) and nucleic acid solution (aqueous) drop by drop, wherein a consistent ratio of lipid-to-nucleic acid is maintained during pipetting while vortexing the mixture. In other embodiments, microfluidic platforms are used to form nanoparticles, e.g. employing the NANOASSEMBLR® IGNITE™ microfluidic instrument (Precision NanoSystems). Exemplary methods of nanoparticle formation are described in John et al., Pharmaceutics. 2024 January; 16(1): 131, Niculescu et al., Int. J. Mol Sci 23(15): 8293 (2022) and in Petersen et al., Eur. J. Pharmaceut. Biopharmaceut. 192: 126-135 (2023), which are incorporated herein in their entireties. In some embodiments, the NLPs produced comprise any of the lipids, or structures, described in Tenchov et al., ACS Nano Vol 15(11), (2021), which is incorporated herein in its entirety.

In some embodiments, the aqueous phase used during nanoparticle assembly comprises a hydrophilic functional agent. When mixed with an organic lipid phase under shear or via microfluidics, vesicle formation results in spontaneous encapsulation of the hydrophilic agent in the NLP's internal aqueous compartment. Hydrophilic core formation is favored when the aqueous-to-organic ratio is between 3:1 and 10:1 and when hydrophilic components are soluble at pH 4-7.

Any of the production methods described herein can be supplemented with any quantitative or qualitative methods known in the art to characterize or identify the NLPs at any step of the production process. NLPs may be characterized by a variety of analysis methods to estimate NLP yield, NLP concentration, NLP purity, NLP composition, or NLP sizes. NLPs can be evaluated by a number of methods known in the art that enable visualization, quantitation, or qualitative characterization (e.g., identification of the composition) of the NLPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify contaminants present on the NLP. To aid in analysis and characterization, of the NLP fraction, the NLPs can additionally be labelled or stained. For example, the NLPs can be stained with 3,3′-dihexyloxacarbocyanine iodide (DIOCe), a fluorescent lipophilic dye, PKH67 (Sigma Aldrich); Alexa Fluor® 488 (Thermo Fisher Scientific), or DyLight™ 800 (Thermo Fisher). For more precise measurements, and to assess the size distributions of NLPs, nanoparticle tracking can be used.

In some embodiments lipids used to form the NLPs are isolated from a variety of lipid sources, using the Bligh-Dyer method (Bligh and Dyer, J Biolchem Physiol, 37: 911-917, 1959). The extracted lipids may be provided as a stock solution, e.g., a solution in chloroform:methanol. Producing the lipid film may comprise, e.g., evaporation of the solvent with a stream of inert gas (e.g., nitrogen).

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ethenpropanol, tetrahydrofura methanol, dimethyl sulfoxide:methanol, or dimethylformamide:methanol. The aqueous phase may be any suitable solution, e.g., a citrate buffer (e.g., a citrate buffer having a pH of about 3.2), water, or phosphate-buffered saline (PBS). The aqueous phase may further comprise a heterologous functional agent, e.g., an agent described in Section II herein, e.g., a nucleic acid (e.g., an siRNA or siRNA precursor (e.g., dsRNA), miRNA or miRNA precursor, mRNA, or plasmid (pDNA)) or a small molecule.

The lipid solution and the aqueous phase may be mixed in the microfluidics device at any suitable ratio. In some examples, aqueous phase and the lipid solution are mixed at a 3:1 volumetric ratio.

### B. Methods of Treating a Plant

### 1) Plants

A variety of plants can be treated with an NLP composition described herein to combat a plant pest, e.g., a microbial plant pest. Plants that can be treated with an NLP composition in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., meristematic tissue, vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple (e.g., ‘Gala’ apple trees), Arabidopsis, banana, barley, canola, castor bean, chrysanthemum, clover, cocoa, coffee, cotton, cottonseed, corn, crambe, cranberry, crucifers, cucumber, Dendrobium, Dioscorea, eucalyptus, fescue, flax, Gladiolus, Liliaceae, linseed, millet, muskmelon, mustard, oat, oil palm, canola or oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat, and vegetable crops such as lettuce, celery, broccoli, cauliflower, cucurbits; fruit and nut trees, such as apple, pear, peach, orange, grapefruit, lemon, lime, pomelo, almond, pecan, walnut, hazel; vines, such as grapes (e.g., a vineyard), kiwi, hops; cannabis, fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis sativa, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculentum, Lycopersicon lycopersicum, Lycopersicon pyriforme), Malus spp., Medicago sativa, Mentha spp., Miscanthus sinensis, Morns nigra, Musa spp., Nicotiana spp., Olea spp., Oryza spp. (e.g., Oryza sativa, Oryza lati folia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solarium integrifolium or Solarium lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

In certain embodiments, the NLP compositions and methods can be used to treat post-harvest plants or plant parts, food, or feed products. In some instances, the food or feed product is a non-plant food or feed product (e.g., a product edible for humans, veterinary animals, or livestock (e.g., mushrooms). When applying the composition comprising an NLP composition comprising a bioactive to a harvested part of a plant (also referred to herein as post-harvest), application may be by a variety of treatment methods, e.g., dip, drip, drench, spray, or fog. In alternative embodiments, the harvested plant part has applied to it a composition, such as a film or membrane, containing NLP composition comprising a bioactive, or is packaged in a container that includes the antimicrobial NLP composition comprising the bioactive. Such treatments, compositions, and containers are further useful for protecting foodstuffs (e.g., processed food products such as bakery goods or processed fruit or vegetables) from fungal growth and spoilage.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. Alternatively, the delivery can occur to a seed. The stages of vegetative and reproductive growth are also referred to herein as “adult” or “mature” plants.

In some embodiments, NLPs with hydrophilic cores are used to encapsulate gene-regulating RNAs, small peptides, or hydrophilic chemical elicitors and deliver them to root, shoot, or meristematic tissues. In some examples, hydrophilic core NLPs allow for co-delivery of RNA and nutrients to support systemic plant responses. In some embodiments, contacting the plant with the NLP composition delivers a hydrophilic functional agent selected from a polynucleotide, peptide, small molecule, metabolite, protein, or biocontrol agent. In some embodiments, the NLP facilitates translocation of the functional agent across plant tissues (e.g., the meristem) or into specific compartments (e.g., apoplast, cytosol, or nucleus), enhancing efficacy compared to unformulated delivery.

### 2) Plant Diseases

Exemplary plant diseases which may be treated or prevented with the NLP compositions described herein, with the causative pathogen shown in parenthesis, include Alternaria Leaf and Fruit Spot (Alternaria alternata), Anthracnose (Colletotrichum acutatum), Leaf Blight (Seimatosporium lichenicola), Leaf Rust (Tranzschelia discolor), Scab (Cladosporium carpophilum), Shot Hole (Wilsonomyces carpophilus), Brown Rot Blossom Blight (Monilinia laxa, M. fructicola), Black Sigatoka (Mycosphaerella fijiensis), Yellow Sigatoka (Mycosphaerella musicola), Alternaria Fruit Rot (Alternaria spp.), Anthracnose Fruit Rot (Colletotrichum gloeosporioides), Botryosphaeria Canker (Botryosphaeria spp.), Leaf Spot and Blotch (Mycosphaerella spp., Septoria spp.), Mummyberry (Monilinia vaccinii-corymbosi), Phomopsis Leaf Spot, Twig Blight and Stem Canker (Phomopsis vaccinii), Powdery Mildew (Sphaerotheca spp.), Septoria Blight (Septoria spp.), Spur Blight (Didymella spp., Phoma spp.), Anthracnose (Sphaceloma necator, Elsinoe veneta), Botryosphaeria Canker (Botryosphaeria dothidea), Colletotrichum Rot (Colletotrichum gloeosporioides), Leaf Spot and Blotch (Mycosphaerella spp., Septoria rubi, Sphaerulina rubi), Powdery Mildew (Sphaerotheca macularis, Microsphaera spp., Oidium spp.), Rosette or Double Blossom of Blackberries (Cercosporella rubi), Spur Blight (Didymella applanata), Blackberry Rust (Phragmidium spp.), Anthracnose (Colletotrichum fragariae), Leather Rot (Phytophthora cactorum), Powdery Mildew (Sphaerotheca macularis), Botrytis grey mould on Foliage (Botrytis cinerea), Seedling Root Rot, Basal Stem Rot (Rhizoctonia solani), Cottonball (Monilinia oxycocci), Fruit Rots (Physalospora vaccinia, Glomerella cingulata, Coleophoma empetri), Lophodermium Twig Blight (Lophodermium spp.), Fairy Ring Suppression (Psilocybe spp.), Albinism (Alternaria alternata pv citri), Alternaria Leaf and Fruit Spot (Alternaria citri), Anthracnose (Colletotrichum acutatum, C. gloeosporioides), Cercospora Leaf Spot (Cercospora spp.), Diplodia Stem-End Rot (Diplodia natalensis), Greasy Spot (Mycosphaerella citri), Melanose (Diaporthe citri), Penicillium Decays, Green Mold, Whisker Mold, Blue Mold (Penicillium spp.), Phomopsis Stem-End Rot (Phomopsis citrii), Post Bloom Fruit Drop (PFD) (Colletotrichum acutatum), Powdery Mildew (Erysiphe spp.), Scab (Elsinoe fawcettii), Sweet Orange Scab (Elsinoe australis), Black Spot (Guignardia citricarpa), Black Rot (Guignardia bidwellii), Downy Mildew (Plasmopara viticola), Phomopsis Cane and Leaf Spot (Phomopsis viticola), Powdery Mildew (Uncinula necator), Botrytis Bunch Rot (Botrytis cinerea), Aspergillus Crown Rot (Aspergillus niger), Pythium Damping Off (Pythium spp.), Stem Rot/White Mold (Sclerotium rolfsii), Rhizoctonia Peg and Pod Rot (Rhizoctonia solani), Stem Rot/White Mold (Sclerotium rolfsii), Cylindrocladium Black Rot (Cylindrocladium crotalariae), Pythium Pod Rot (Pythium myriotylum), Alternaria Late Blight (Alternaria alternata), Botryosphaeria Panicle and Shoot Blight (Botryosphaeria dothidea), Septoria Leaf Spot (Septoria pistaciarum), Scab (Cladosporium carpophilum), Alternaria Spot and Fruit Rot (Alternaria alternata), Anthracnose (Colletotrichum prunicola, C. gloeosporioides), Leaf Rust (Tranzschelia discolor), Powdery Mildew (Sphaerotheca pannosa, Podosphaera clandestina), Shot Hole (Wilsonomyces carpophilus), Alternaria Leaf Spot (Alternaria spp., A. alternata), Ascochyta Leaf Spot (Ascochyta cynarae), Phyllosticta Leaf Spot (Phyllosticta spp.), Rust (Uromyces betae, Puccinia helianthi), White Rust (Albugo tragopogonis), Anthracnose (Colletotrichum acutatum, Glomerella cingulata), Eastern Filbert Blight (Anisogramma anomale), Late Blight (Alternaria alternata), Scab (Cladosporium carpophilum), Septoria Leaf Spot (Septoria pistaciarum), Shot Hole (Wilsonomyces carpophilus), Blossom Blight (Monilinia laxa, M. fructicola), Powdery Mildew (Erysiphe spp.), Rust (Puccinia spp.), Alternaria black spot (Alternaria brassicae), Black leg/Phoma (Leptosphaeria maculans), Cercospora leaf spot (C. beticola), Head rot (Rhizoctonia solani), Leaf spot and pod rot (Alternaria alternata), Powdery mildew (Erysiphe polygoni), Southern blight (Sclerotium rolfsii), Anthracnose leaf blight (Colletotrichum graminicola), Gray leaf spot (Cercospora sorghi), Northern corn leaf blight (Setosphaeria turcica), Northern corn leaf spot (Cochliobolus carbonum), Common Rust (Puccinia sorghi), Southern Rust (P. polysora), Southern corn leaf blight (Cochliobolus heterostrophus), Eye spot (Aureobasidium zeae), Physoderma brown spot (P. maydis), Yellow Leaf Blight (Phyllosticta maydis), Ascochyta blight (A. gossypii), Rust (Puccinia schedonnardi, P. cacabata), Rhizoctonia leaf and stem diseases (R. solani), Target spot (Corynespora cassiicola), Southern blight (Sclerotium rolfsii), Rhizoctonia limb rot (R. solani), Cylindrocladium black rot (C. crotalaria), White mold (Sclerotinia minor), Early leaf spot (Cercospora arachidicola), Late leaf spot (Cercosporidium personatum), Web blotch (Phoma arachidicola), Rust (Puccinia arachidis), Pepper Spot (Leptosphaerulina crassiasca), Southern stem rot (Sclerotium rolfsii), Rhizoctonia limb rot (R. solani), Cylindrocladium black rot (C. crotalaria), White mold (Sclerotinia minor), Anthracnose (Colletotrichum lindemuthianum), Ascochyta blight (A. phaseolorum), Cercospora leaf blotch (C. cruenta), Downy mildew (Phytophthora nicotianae), Rust (Uromyces appendiculatus), Anthracnose (ripe rot) (C. gloeosporioides), Mummy berry (M. vacciniicorymbosi), Rust (Pucciniastrum vaccinii), Septoria leaf spot (Septoria albopunctata), Downy mildew (Peronospora parasitica), Alternaria leaf blight (A. dauci), Cercospora leaf spot (C. carotae), Basal stalk rot (Rhizoctonia solani), Early blight (Cercospora apii), Late blight (Septoria apicola), Verticihium brown spot and dry bubble, Pink rot (Sclerotinia sclerotiorum), Lophodermium leaf/twig blight (hypophyllum), Upright dieback (Phomopsis vaccinii), Anthracnose (Colletotrichum spp.), Downy mildew (Pseudoperonospora cubensis), Target spot (Corynespora cassiicola), Alternaria leaf blight (A. cucumerina), Alternaria leaf spot (A. alternata), Cercospora leaf spot (C. citrullina), Gummy stem blight/vine decline (Didymella bryoniae), Powdery mildew (Sphaerotheca only), Scab (Cladosporium cucumerinum), Anthracnose (Colletotrichum spp.), Botrytis leaf mold (Botrytis cinerea), Cercospora leaf spot (Cercospora spp.), Powdery mildew (Leveillula taurica), Purple blotch (Alternaria porri), Botrytis neck rot, Downy mildew (Peronospora destructor), Early leaf spot (Cercospora arachidicola), Late leaf spot (Cercosporidium personatum), Pepper spot (Leptosphaerulina crassiasca), Black dot (Colletotrichum coccodes), Botrytis vine rot (B. cinerea), Early blight (Alternaria solani), Late blight (Phytophthora infestans), Anthracnose (Colletotrichum truncatum), Cercospora leaf blight (C. kikuchii), Diaporthe pod and stem rot (D. phaseolorum), Frogeye leaf spot (Cercospora sojina), Purple seed stain (C. kikuchii), Septoria brown spot (S. glycines), Rust (Phakopsora pachyrhizi), Stem canker (Diaporthe phaseolorum), Early blight (Alternaria solani), Gray leaf mold (Fluvia fluva Cladosporium), Gray leaf spot (Stemphylium botryosum), Late blight (Phytophthora infestans), Septoria leaf spot (S. lycopersici), Target spot (Corynespora cassiicola), Alternaria fruit rot (black mold) (A. alternata), Anthracnose (Colletotrichum spp.), Botrytis gray mold (B. cinerea), Late blight fruit rot (P. infestans), Rhizoctonia fruit rot (R. solani), Anthracnose (Colletotrichum gloeosporioides), Anthracnose (Colletotrichum acutatum), Blossom blight/brown rot (Monilinia spp.), Scab (Venturia carpophila), Shot hole (Wilsonomyces carpophilus), Leaf curl (Taphrina deformans), Black knot (cherry, plum) (Apiosporina morbosa), Cherry leaf spot (Blumeriella jaapii), Scab (Cladosporium carpophilum), Interior needle blight (Mycosphaerella spp. and Phaeocryptopus nudus), Swiss needlecast (Phaeocryptopus gaeumannii), Interior needle blight (Mycosphaerella spp. and Phaeocryptopus nudus), Scleroderris canker (Gremmeniella abietina), Leaf rust (Thekopsora minima), Powdery mildew (Erysiphe necator), Alternaria rot (A. alternata), Angular leaf spot (Mycosphaerella angulata), Anthracnose (Elsinoe ampelina), Black Rot (Guignardia bidwellii), Leaf Blight (Pseudocercospora vitis), Phomopsis cane and leaf spot (P. viticola), Rotbrenner (Pseudopezicula tracheiphila), Septoria leaf spot (S. ampelina), Apple Scab (Venturia inaequalis), Pear Scab (V. piris), Alternaria blotch, Alternaria rot (Alternaria spp.), Cedar apple rust (Gymnosporangium juniper-virginianae), Powdery mildew (Podosphaera leucotricha), Quince rust (Gymnosporangium spp.), Flyspeck and Sooty blotch, Bitter rot (Glomerella cingulata), Black rot (Botryosphaeria obtusa), Brooks fruit spot (Mycosphaerella pomi), White rot (Botryosphaeria dothidea), Alternaria rot and surface mold, Bitter rot, Blue mold, Bull's-eye rot, Gray mold, Phacidiopycnis rot, Rhizopus rot, Speck rot, Sphaeropsis rot, White rot, Damping off (Pythium spp.), Root Rot (Phytophthora spp.), Leather rot (P. cactorum), Red stele (P. fragariae), Vascular collapse (P. cactorum), Basal stem rot (Phytophthora spp.), Crown rot (Phytophthora capsici), Downy Mildew (Peronospora effuse, P. farinosa), White rust (Albugo occidentalis), Pink rot (Phytophthora erythroseptica), Pythium leak, Pythium seedling disease (Pythium spp.), Phytophthora root and stem rot (Phytophthora megasperma), Pythium damping off (Pythium spp.), Collar rot, Crown rot, Root rot (Phytophthora spp.), Crown rot, Spear rot (Phytophthora spp.), Root Rot (Phytophthora cinnamomi), Downy mildew (Peronospora parasitica), Brown rot, Citrus foot rot, Gummosis, Root rot, Trunk canker (Phytophthora spp.), or Downy mildew (Bremia lactucae). From an agricultural or horticultural perspective, and for the purposes of this application, some of the pathogens and diseases listed above are considered “fungal” although the causative pathogen is technically an oomycete (phylum Oomycota), including, but not limited to Pythion spp., Phytophthora spp., Peronospora spp., Plasmopara spp., Albugo spp., and Bremia spp.

### 3) Means of Contacting a Plant

In some embodiments, the disclosure provides methods for contacting the plant with the NLP composition described herein, e.g., topically contacting the plant with the NLP compositions described herein. Administration generally is achieved by application of the compositions in a vehicle compatible with the plant to be treated (e.g., a botanically compatible vehicle or carrier), such as an aqueous vehicle, to the plant or to the soil surrounding the plant or by injection into the plant. Any application can be used; however, one application method includes trunk injection and foliar spraying as described herein. Other methods include application to the soil surrounding the plant, by injection, soaking or spraying, so that the applied compounds can come into contact with the plant roots and can be taken up by the roots. Additional topical applications may also be contemplated. The compositions disclosed herein can be formulated for seed or plant treatments in any of the following modes: dry powder, water slurriable powder, liquid solution, flowable concentrate or emulsion, emulsion, microcapsules, gel, or water dispersible granules.

In some aspects, the methods described herein comprise detecting the biodistribution of the NLPs administered to plants (e.g., detecting distribution throughout the trunk, stem, and other parts of a plant). Various means may be used for detection of biodistribution in a plant. In some embodiments, the NLPs described herein comprise a fluorescent label for use in detecting biodistribution of the NLP compositions, e.g., an encapsulated dye or a fluorescently labeled heterologous functional agent.

Delivery of the NLP composition to the plant or plant part can be via different routes. The compositions can be suitably administered as an aerosol, for example by spraying onto leaves or other plant material. The particles can also be administered by injection, for example directly into a plant, such as into the stem. In certain embodiments the compositions are administered to the roots. This can be achieved by spraying or watering plant roots with compositions. In other embodiments, the particles are introduced into the xylem or phloem, for example by injection or being included in a water supply feeding the xylem or phloem. Application to the stems or leaves of the plant can be performed by spraying or other direct application to the desired area of the plant; however, any method known in the art can be used. A solution or vehicle containing NLPs at a dosage of active ingredient can be applied with a sprayer to the stems or leaves until runoff to ensure complete coverage, and repeat three or four times in a growing season. The concentrations, volumes and repeat treatments may change depending on the plant.

In certain embodiments, the method of delivery comprises precision delivery (also referred to as “precision injection”) of a formulation into a plant, e.g. a citrus plant. Precision delivery refers to delivering the formulation only or substantially only into a target location in the plant. For example, in some embodiments, the target location is the active vasculature of the plant. In certain embodiments, the method comprises injecting an injection formulation into and no further than the active vasculature of the plant. In some embodiments, the composition enters the active vasculature and is transported throughout the plant. In some variations, the active vasculature of the plant is the xylem and/or the phloem. In one variation, the active vasculature is active xylem (such as sapstream) and phloem. In further embodiments, precision delivery involves delivering the formulation into the active vasculature of the plant while minimizing damage to the plant relative to traditional forms of injection drilling systems. In yet other embodiments, precision delivery involves using a system that can be configured to deliver formulation into and no further than the active vasculature of a plant. Exemplary injection technologies are disclosed in WO2020021041, which is incorporated in its entirety herein.

In some embodiments, the injection systems comprise an injection tool, a fluid delivery unit, and an injection formulation source. In operation, the injection tool is operatively connected to the fluid delivery unit such that injection formulation flows from the source through the injection tool into the plant. In some embodiments, the source of injection formulation is independent of the fluid delivery unit. In other embodiments, the source of injection formulation is integral with the fluid delivery unit. In some embodiments, the injecting of the injection formulation comprises delivering at least a portion of the injection formulation from the fluid delivery unit through the injection tool into and no further than the active vasculature of the plant.

In some embodiments, this disclosure also provides systems and devices for delivering injection formulations to the interior of the plant. In some embodiments, the systems comprise an injection tool operatively connected to a fluid delivery unit, wherein the injection tool is configured for precision delivery of the injection formulation to a target location inside the plant. In some embodiments, the systems are configured for precision delivery of an injection formulation into the active vasculature of a plant. In some embodiments, the fluid delivery unit further comprises the formulation. In other embodiments, the system comprises an injection tool, a fluid delivery unit, and a source of source of formulation in fluid communication with the fluid delivery unit.

In some variations, the body is shaped to pierce the plant, such as the trunk or stem of the plant. In certain variations, the body is in the shape of a blade. In certain variations, the body has a cutting edge at the tip of the body, and the width of the cutting edge is narrower than width of the body in the area connected to the base. In certain variations, the body comprises: at least one outlet that receives the injection formulation from the at least one inlet, and at least one distribution reservoir that retains the injection formulation proximate to adjacent tissue of the plant. In certain variations, the fluid delivery unit is configured to store and deliver the injection formulation. In certain variations, the fluid delivery unit comprises a pressurized container (e.g., a pressurized canister).

In some variations, the method of injection delivers at least 50%, at least 60%, at least 70%, at least 80%, at least 90%, at least 95%, at least 96%, at least 97%, at least 98% or at least 99% of the injection formulation into to the active vasculature of the plant. In one variation, the methods deliver at least 50%, at least 60%, at least 70%, at least 80%, at least 90%, at least 95%, at least 96%, at least 97%, at least 98% or at least 99% of the injection formulation into the xylem and/or phloem of the plant.

### 4) Method of Increasing Plant Fitness

In one aspect, provided herein is a method of increasing the fitness of a plant, the method including delivering to the plant the NLP composition described herein (e.g., in an effective amount and duration) to increase the fitness of the plant relative to an untreated plant (e.g., a plant that has not been delivered the NLP composition).

An increase in the fitness of the plant as a consequence of delivery a heterologous functional agent in an NLP composition described in any of the embodiments herein to a plant can manifest in a number of ways, e.g., thereby resulting in a better production of the plant, for example, an improved yield, improved vigor of the plant (e.g., improved tolerance of abiotic or biotic stress or improved resistance to pests) or improved quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional agricultural agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a NLP composition described herein can also be measured by other methods, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional agricultural agents.

Provided herein is a method of modifying or increasing the fitness of a plant, the method including delivering to the plant an effective amount of an NLP composition provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield underwater-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g., nematode stress, insect herbivory stress, fungal pathogen stress, bacterial pathogen stress, or viral pathogen stress. The stress may be temporary, e.g., several hours, several days, several months, or permanent, e.g., for the life of the plant.

In some variations, treatment of a plant with an NLP composition provided herein can (i) reduce fruit drop; (ii) increase Brix in the fruit; and/or (iii) increase fruit yield. In certain variations, the treatment protocols provided herein can (i) reduce fruit drop by at least 10%, at least 15%, at least 20%, or at least 25%, or between 5% and 50%, between 5% and 40%, between 10% and 30%, or between 15% and 25%; (ii) increase Brix by at least 1%, or at least 5%, or between 1% and 10%; and/or (iii) increase yield by at least 25%, at least 30%, at least 35%, at least 40%, at least 45%, or at least 50%, or between 25% and 75%, or between 40% and 60%. Overall, in one variation, the treatment protocols provided herein can improve recovery of plant health, and yield a healthier, more resilient grove.

In some variations, the average fruit drop for the plants to which the injection formulation is administered is less than 25, less than 20, less than 15 or less than 10; or between 10 and 25. In some variations, the average fruit yield for the plants to which the injection formulation is administered is at least 35 lbs, at least 40 lbs, at least 45 lbs, at least 50 lbs, at least 55 lbs, at least 60 lbs, at least 65 lbs, at least 70 lbs, at least 75 lbs, at least 80 lbs, or least 85 lbs per plant, or between 30 and 90 lbs, or between 35 and 85 lbs per plant.

In some variations, an average Brix for the plants to which the injection formulation is administered is at least 7.5, at least 8, or at least 8.5; or between 7 and 9, or between 7.5 and 8.5.

In some instances, the increase in plant fitness is an increase (e.g., by about 1%, 2%, 5%, 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait of a plant that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of an NLP composition provided herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of an NLP composition disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount an NLP composition provided herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of an rLP composition provided herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of an NLP composition provided herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of an NLP composition provided herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In cases where an herbicide is included with the NLP composition, the methods may be further used to decrease the fitness of or kill weeds. In such instances, the method may be effective to decrease the fitness of the weed by about 2%, 5%, 10%, 20%, 30%, 40%, 50%, 60%, 70%, 80%, 90%, 100%, or more in comparison to an untreated weed (e.g., a weed to which the NLP composition has not been administered). For example, the method may be effective to kill the weed, thereby decreasing a population of the weed by about 2%, 5%, 10%, 20%, 30%, 40%, 50%, 60%, 70%, 80%, 90%, 100%, or more in comparison to an untreated weed. In some instances, the method substantially eliminates the weed. Examples of weeds that can be treated in accordance with the present methods are further described herein.

In some embodiments, the methods described herein comprise the use of NLPs having a hydrophilic core to deliver functional agents to plant pests, including but not limited to insects, fungi, bacteria, and nematodes. The NLPs may be applied to the plant surface, soil, or root zone, or may be introduced into plant tissues systemically, such that the pest is exposed to the encapsulated agent through ingestion, contact, or colonization of the treated plant. In these embodiments, the hydrophilic core of the NLP encapsulates a water-soluble or polar functional agent with pesticidal, antifungal, or anti-nematodal activity. Examples of such agents include: Insecticidal peptides or proteins, such as Bt toxins, protease inhibitors, or ribosome-inactivating proteins; RNA-based agents, such as double-stranded RNA (dsRNA), small interfering RNA (siRNA), or antisense oligonucleotides that silence essential genes in insect or fungal pests; Hydrophilic antibiotics or antifungals, such as streptomycin, oxytetracycline, or fosetyl-aluminum; Elicitors or signaling compounds, such as chitosan derivatives or -glucans, that trigger plant defenses against pathogens. The use of a hydrophilic-core NLP enhances the solubility, stability, and protection of such agents from enzymatic degradation or environmental breakdown prior to reaching the target pest. In some embodiments, the NLP facilitates systemic movement of the functional agent within the plant, thereby allowing delivery to feeding or infecting pests located in remote tissues. In other embodiments, the NLP remains localized at the application site, forming a depot for sustained release. In other embodiments, the NLP facilitates uptake of the functional by the plant pest. The method may be used prophylactically or therapeutically, and may be applied alone or in combination with other agricultural agents. Improved efficacy may be achieved through optimized NLP size (e.g., <200 nm), surface charge, or inclusion of targeting lipids or surface ligands.

### EXAMPLES

The following are examples of the methods of the invention. It is understood that various other embodiments may be practiced, given the general description provided above.

### Example 1. Composition and Physicochemical Characteristics of NLPs Produced

This Example describes 1) constituents of NLPs; 2) physicochemical characteristics of NLPs produced; 3) composition of NLPs produced and 4) cargo in NLPs produced.

### Experimental Procedure

### a) NLP Constituents

The phospholipids, sterols, synthetic lipids, and pegylated lipids used to make the NLPs are presented in Table 1.

TABLE 1
NLP constituents

| NLP constituent | Name |
|---|---|
| Phospholipids | DOPE |
| | DSPC |
| | Soybean Lecithin (de-oiled) |
| | Sunflower Lecithin (de-oiled) |
| | MGDG |
| | Hydro Soy PC |
| Sterols | Cholesterol |
| | Sitoesterol |
| Synthetic lipids | DDAB |
| | DOTAP |
| | SM-102 |
| | MC3 |
| | KC2 |
| | ALC-315 |
| | DOTAP |
| | DODMA |
| | DODAP |
| Pegylated lipids | PEG5000-PE 14:0 |
| | PEG2000-PE 14:0 |
| | PEG2000-PE 18:0 |
| NLP cargo | Rho-DHPE (dye) |
| | GFP-mRNA |
| | NLS-mGFP-RNA |
| | Nano luciferase (Nluc) mRNA |
| | siRNA-GFP |
| | siRNA-Magnesium chelatase (MgChel) |

### b) NLPs are Produced by any of the Methods Presented in Example 4.

Tables 2, 3, 4, 5, 6 and 7 show the physicochemical characteristics, lipid compositions and cargos in three NLPs batches produced. Table 2 shows the physicochemical characteristics and lipid compositions of a first batch of NLPs produced. Table 3 shows the cargo in the first batch of NLPs produced. Table 4 shows the physicochemical characteristics and lipid compositions of a second batch of NLPs produced. Table 5 shows the cargo in the second batch of NLPs produced. Table 6 shows the physicochemical characteristics and lipid compositions of a third batch of NLPs produced. Table 7 shows the cargo in the third batch of NLPs produced.

NLP formation was verified by electron and cryo-electron microscopy on a JEOL 1010 transmission electron microscope, following the protocol from Wu et al., Analyst. 140(2):386-406, 2015. The NLP particle size, NLP particle size distribution, and zeta potential were measured using a Malvern Zetasizer, following the manufacturer's instructions.

The molar percentages (mol %) of the various constituents are indicated. The mol % are calculated first by calculating the moles of every component, e.g. a lipid, in the formulation by taking into account the amount of lipid added into the formulation (uL) and the concentration of the stock solution (e.g., 5 ug/uL) and the molecular weight of the corresponding lipid. Then the mole % is obtained taking into account the moles of every component (e.g. the lipid) in the formulation with the total number of moles of all liposome constituents combined in the specific formulation.

Incorporation of polynucleotide in the NLPs was confirmed by quantifying RNA concentration by Quant-it™ RiboGreen RNA Assay Kit (Invitrogen) according to manufacturer's protocol. The Quant-iT™ RiboGreen™ RNA method is a highly sensitive and specific fluorescent assay for quantifying RNA in solution. It utilizes the RiboGreen™ dye, which selectively binds to RNA, producing a strong fluorescence signal proportional to the RNA concentration. This method allows for accurate RNA measurement even in the presence of contaminants like DNA and proteins.

The concentration of cargo is expressed per ml of NLP suspension. The average particle size, PDI, zeta potential were measured using a Malvern Panalytical Zetasizer Ultra Red Label. immediately after production. The encapsulation efficiency was calculated by the (initial concentration of polynucleotide added/actual concentration of polynucleotide in the NLP)*100%, immediately after production.

TABLE 2
Physicochemical characteristics and lipid composition
(molar percentages) of a first batch of NLPs produced

| | | Size | | Zeta pot. | | | | SF |
|---|---|---|---|---|---|---|---|---|
| Formulation | ID | (nm) | PDI | (mV) | Chol | DDAB | DOPE | lecithin |
| FORM 27 | rNLP_H1 | 252.8 | 0.149 | 13.66 | 33.68 | 20.67 | 28.47 | 17.18 |
| FORM 34 | rNLP_H2 | 132.3 | 0.1531 | 20.94 | 0.04 | 0.04 | 0.065 | 0.04 |
| FORM 29 | rNLP_H3 | 338.9 | 0.09 | 13.98 | 34.35 | 21.08 | 24.56 | 17.52 |
| FORM 30 | rNLP_H4 | 196.8 | 0.189 | 24.92 | 34.35 | 21.08 | 24.56 | 17.52 |
| FORM 32 | rNLP_H5 | 142.6 | 0.1136 | −30.44 | 0.1 | 0.01 | 0.035 | 0.04 |
| FORM 33 | rNLP_H6 | 127.2 | 0.1546 | −40.13 | 0.1 | 0.01 | 0.035 | 0.04 |
| FORM 31 | rNLP_H7 | 199.5 | 0.31 | 31.28 | 35.95 | 22.06 | 21.04 | 18.34 |
| FORM 45 | rNLP_H8 | 846.6 | 0.826 | −38.88 | 0.095 | 0.04 | 0.065 | 0.04 |
| FORM 46 | rNLP_H9 | 524.9 | 0.611 | n.d. | 0.095 | 0.04 | 0.065 | 0.04 |
| FORM 48 | rNLP_H10 | n.d.¹ | n.d. | n.d. | 0.095 | 0.01 | 0.065 | 0.04 |
| FORM 49 | rNLP_H11 | 412.8 | 0.233 | 12.72 | 34.4 | 21.11 | 15.66 | 26.33 |
| FORM 50 | rNLP_H12 | 635.2 | 0.318 | 11.76 | 34.46 | 21.15 | 6.72 | 35.16 |

¹n.d., not determined

TABLE 3
Cargo in the first batch of NLPs

| Form- | | Rho- | GFP-RNA | Nluc RNA | Encapsulation |
|---|---|---|---|---|---|
| ulation | ID | DPE | (ug/ml) | (ug/ul) | efficiency (%) |
| FORM 27 | rNLP_H1 | | 20 | — | 83.64 |
| FORM 34 | rNLP_H2 | 2.38 | — | 20 | 95.61 |
| FORM 29 | rNLP_H3 | 2.49 | 20 | — | 93.07 |
| FORM 30 | rNLP_H4 | 2.49 | — | — | n/a¹ |
| FORM 32 | rNLP_H5 | 2.38 | — | 20 | n.d.² |
| FORM 33 | rNLP_H6 | 1.96 | — | 100 | n.d. |
| FORM 31 | rNLP_H7 | 2.61 | 100 | — | 73.1 |
| FORM 45 | rNLP_H8 | | — | 20 | 59.15 |
| FORM 46 | rNLP_H9 | 1.64 | — | 20 | 27.89 |
| FORM 48 | rNLP_H10 | 1.83 | — | 20 | 33.19 |
| FORM 49 | rNLP_H11 | 2.49 | 20 | — | 94.8 |
| FORM 50 | rNLP_H12 | 2.56 | 20 | — | 90.53 |

¹n/a, not applicable
²n.d., not determined

TABLE 4
Physicochemical characteristics and lipid composition
(molar percentages) of a second batch of NLPs produced

| | | | | Zeta | | | | | | |
|---|---|---|---|---|---|---|---|---|---|---|
| | | Size | | pot. | Choles- | Sistos- | | | ALC- | |
| Formulation | ID | (nm) | PDI | (mV) | terol | terol | DDAB | DOTAP | 315 | DOPE |
| FORM 47 | rNLP_I1 | 309.2 | 0.231 | 42.27 | 0.095 | — | 0.04 | — | — | 0.065 |
| FORM 54 | rNLP_I2 | 167.2 | 0.066 | −0.134 | 34.15 | — | 20.96 | — | — | 24.42 |
| FORM 55 | rNLP_I3 | 178.5 | 0.073 | −0.03 | 33.93 | — | 20.82 | — | — | 24.26 |
| FORM 56 | rNLP_I4 | n.d.¹ | n.d. | n.d. | 0.04 | — | | — | 0.04 | 0.055 |
| FORM 57 | rNLP_I5 | n.d. | n.d. | n.d. | 0.04 | — | | — | 0.04 | 0.055 |
| FORM 58 | rNLP_I6 | 776.7 | 0.134 | 29.86 | 34.67 | — | 15.96 | — | 4.37 | 24.8 |
| FORM 59 | rNLP_I7 | 847.7 | 0.094 | 28.99 | 35 | — | 10.74 | — | 8.83 | 25.03 |
| FORM 60 | rNLP_I8 | 181.4 | 0.130 | −0.015 | 33.4 | — | 20.49 | — | — | 26.05 |
| FORM 61 | rNLP_I9 | 336.7 | 0.120 | 4.820 | 32.98 | — | 20.24 | — | — | 27.45 |
| FORM 62 | rNLP_I10 | 224.8 | 0.310 | −0.096 | 33.93 | — | 20.82 | — | — | 24.26 |
| FORM 63 | rNLP_I11 | 176.6 | 0.200 | −0.970 | 33.28 | — | 20.42 | — | — | 28.13 |
| FORM 64 | rNLP_I12 | 185.4 | 0.039 | 36.7 | 37.69 | — | 49.77 | — | — | — |
| FORM 65 | rNLP_I13 | 313.6 | 0.667 | 5.081 | 35.01 | — | 21.49 | — | — | 25.04 |
| FORM 66 | rNLP_I14 | 341.2 | 0.6102 | 3.82 | 35.01 | — | 21.49 | — | — | 25.04 |
| FORM 74 | rNLP_I15 | 235.4 | 0.87 | 8.22 | 35.01 | — | 21.49 | — | — | 25.04 |
| FORM 75 | rNLP_I16 | 232.7 | 0.248 | 8.34 | 34.33 | — | 21.07 | — | — | 26.79 |
| FORM 76 | rNLP_I17 | 208.4 | 0.221 | 15.1 | 33.81 | — | 20.75 | — | — | 28.14 |
| FORM 78 | rNLP_I18 | 180.6 | 0.233 | 10.59 | 35.22 | — | 21.62 | — | — | 25.19 |
| FORM 79 | rNLP_I19 | 96.26 | 0.213 | 20.73 | 42.63 | — | 26.16 | — | — | 30.49 |
| FORM 83 | rNLP_I20 | 156.2 | 0.5384 | 5.77 | 35 | — | 21.5 | — | — | 25 |
| FORM 84 | rNLP_I21 | 176.9 | 0.58 | NA | 33.36 | — | 20.48 | — | — | — |
| FORM 85 | rNLP_I22 | 167.2 | 0.5 | NA | 31.99 | — | 19.63 | — | — | — |
| FORM86 | rNLP_I23 | 124.2 | 0.448 | 10.27 | 35.77 | — | — | 19.8 | — | 25.58 |
| FORM 113 | rNLP_I25 | 200.9 | 0.154 | 0.774 | 42.63 | — | 26.16 | — | — | 30.49 |
| FORM 130 | rNLP_I26 | 72.32 | 0.2132 | 42.92 | — | 38.5 | — | 49 | — | — |
| FORM 131 | rNLP_I27 | 180 | 0.2269 | 8.056 | — | 27 | — | — | — | — |
| FORM 132 | rNLP_I28 | 579.6 | 0.5594 | 12.88 | — | 32.9 | — | 41.9 | — | — |
| FORM 133 | rNLP_I29 | 157.2 | 0.234 | 1.639 | — | 22.9 | — | | — | — |

| | | | | | PEG5K | PEG2K | PEG2K | | |
|---|---|---|---|---|---|---|---|---|---|
| Formulation | ID | DODMA | DODAP | DSPC | PE 14:0 | PE 14:0 | PE 18:0 | SB lec | SF lec |
| FORM 47 | rNLP_I1 | — | — | — | — | — | — | — | 0.04 |
| FORM 54 | rNLP_I2 | — | — | — | 0.58 | — | — | — | 17.42 |
| FORM 55 | rNLP_I3 | — | — | — | — | 1.22 | — | — | 17.3 |
| FORM 56 | rNLP_I4 | — | — | — | — | — | — | — | 0.04 |
| FORM 57 | rNLP_I5 | — | — | — | — | — | — | — | 0.04 |
| FORM 58 | rNLP_I6 | — | — | — | — | — | — | — | 17.69 |
| FORM 59 | rNLP_I7 | — | — | — | — | — | — | — | 17.86 |
| FORM 60 | rNLP_I8 | — | — | — | — | 0.6 | — | — | 17.04 |
| FORM 61 | rNLP_I9 | — | — | — | — | 0.12 | — | — | 16.83 |
| FORM 62 | rNLP_I10 | — | — | — | — | 1.22 | — | — | 17.31 |
| FORM 63 | rNLP_I11 | — | — | — | — | 1.19 | — | — | 16.98 |
| FORM 64 | rNLP_I12 | — | — | 10.59 | — | — | 1.59 | — | |
| FORM 65 | rNLP_I13 | — | — | — | 0.6 | — | — | — | 17.86 |
| FORM 66 | rNLP_II4 | — | — | — | 0.6 | — | — | — | 17.86 |
| FORM 74 | rNLP_I15 | — | — | — | 0.6 | — | — | — | 17.86 |
| FORM 75 | rNLP_I16 | — | — | — | 0.29 | — | — | — | 17.52 |
| FORM 76 | rNLP_I17 | — | — | — | 0.057 | — | — | — | 17.25 |
| FORM 78 | rNLP_I18 | — | — | — | — | — | — | — | 17.97 |
| FORM 79 | rNLP_I19 | — | — | — | 0.72 | — | — | — | — |
| FORM 83 | rNLP_I20 | — | — | — | 0.6 | — | — | 17.9 | — |
| FORM 84 | rNLP_I21 | 28.57 | — | — | 0.57 | — | — | — | 17.02 |
| FORM 85 | rNLP_I22 | — | 31.51 | — | 0.54 | — | — | — | 16.32 |
| FORM86 | rNLP_I23 | — | — | — | 0.61 | — | — | — | 18.25 |
| FORM 113 | rNLP_I25 | — | — | — | 0.72 | — | — | — | — |
| FORM 130 | rNLP_I26 | — | — | 11 | — | — | 1.5 | — | — |
| FORM 131 | rNLP_I27 | 52 | — | 15 | — | — | 6 | — | — |
| FORM 132 | rNLP_I28 | — | — | 9.4 | — | — | 1.28 | — | 14.6 |
| FORM 133 | rNLP_I29 | 44.1 | — | 12.7 | — | — | 5.09 | — | 15.2 |

¹n.d., not determined

TABLE 5
Cargo in the second batch of NLPs

| Form- | | Rho- | GFP-RNA | Nluc RNA | Encapsulation |
|---|---|---|---|---|---|
| ulation | ID | DPE | (ug/ml) | (ug/ul) | efficiency (%) |
| FORM 47 | rNLP_I1 | 1.64 | — | 20 | 62.96 |
| FORM 54 | rNLP_I2 | 2.48 | 20 | — | 92.92 |
| FORM 55 | rNLP_I3 | 2.46 | 20 | — | 92.72 |
| FORM 56 | rNLP_I4 | 2.59 | — | — | n.d.¹ |
| FORM 57 | rNLP_I5 | 2.59 | 20 | — | n.d. |
| FORM 58 | rNLP_I6 | 2.51 | 20 | — | 90.77 |
| FORM 59 | rNLP_I7 | 2.54 | 20 | — | 91.29 |
| FORM 60 | rNLP_I8 | 2.42 | 20 | — | 92.17 |
| FORM 61 | rNLP_I9 | 2.39 | 20 | — | 89.25 |
| FORM 62 | rNLP_I10 | 2.46 | — | — | n/a² |
| FORM 63 | rNLP_I11 | — | 20 | — | 37.14 |
| FORM 64 | rNLP_I12 | — | 20 | — | 82.18 |
| FORM 65 | rNLP_I13 | — | 20 | — | 93.64 |
| FORM 66 | rNLP_I14 | — | — | — | n/a |
| FORM 74 | rNLP_I15 | — | 20 | — | 91.4 |
| FORM 75 | rNLP_I16 | — | 20 | — | 93.01 |
| FORM 76 | rNLP_I17 | — | 20 | — | 93.34 |
| FORM 78 | rNLP_I18 | — | 20 | — | 92.54 |
| FORM 79 | rNLP_I19 | — | 20 | — | 95.38 |
| FORM 83 | rNLP_I20 | — | 20 | — | 90.74 |
| FORM 84 | rNLP_I21 | — | 20 | — | 94.97 |
| FORM 85 | rNLP_I22 | — | 20 | — | 95.37 |
| FORM 86 | rNLP_I23 | — | 20 | — | 88.58 |
| FORM 113 | rNLP_I25 | — | 20 | — | 78.69 |
| FORM 130 | rNLP_I26 | — | 20 | — | 97.8 |
| FORM 131 | rNLP_I27 | — | 20 | — | 42.89 |
| FORM 132 | rNLP_I28 | — | 20 | — | 94.05 |
| FORM 133 | rNLP_I29 | — | 20 | — | 51.63 |

¹n.d, not determined
²n/a, not applicable

TABLE 6
Physicochemical characteristics and lipid composition
(molar percentages) of third batch of NLPs

| | | Size | | Zeta pot. | | | SM- | | |
|---|---|---|---|---|---|---|---|---|---|
| Formulation | ID | (nm) | PDI | (mV) | Chol | DDAB | 102 | MC3 | KC2 |
| FORM 114 | rNLP_S1 | 181.8 | 0.175 | 1.24 | 30 | 50 | — | — | — |
| FORM 115 | rNLP_S2 | 444.2 | 0.217 | 8.5 | 35.22 | 21.62 | — | — | — |
| FORM 116 | rNLP_S3 | 160.8 | 0.249 | 1.81 | 30 | 50 | — | — | — |
| FORM 117 | rNLP_S4 | 227 | 0.193 | 4.94 | 42.29 | 25.95 | — | — | — |
| FORM 118 | rNLP_S5 | 301.2 | 0.143 | 7.41 | 35.22 | 21.62 | — | — | — |
| FORM 119 | rNLP_S6 | 431.9 | 0.073 | 35.4 | 35.48 | 21.78 | — | — | — |
| FORM 120 | rNLP_S7 | 289.5 | 0.106 | 19.3 | 38.5 | — | — | 50 | — |
| FORM 121 | rNLP_S8 | 239 | 0.143 | 4.57 | 27.6 | 67.2 | — | — | — |
| FORM 122 | rNLP_S9 | 109.5 | 0.219 | 5.92 | 38.5 | — | — | 50 | — |
| FORM 123 | rNLP_S10 | 286.7 | 0.046 | 8.23 | 33.7 | 56.4 | — | — | — |
| FORM 124 | rNLP_S11 | 228.9 | 0.054 | 8.72 | — | — | — | — | — |
| FORM 125 | rNLP_S12 | 385.5 | 0.055 | 12.2 | 48.91 | 60.09 | — | — | — |
| FORM 126 | rNLP_S13 | 96.97 | 0.201 | n.d.¹ | 20.83 | — | — | — | 75.26 |
| FORM 127 | rNLP_S14 | 94.29 | 0.181 | 4.02 | 11.88 | — | — | — | 85.88 |
| FORM 128 | rNLP_S15 | 206 | 0.277 | n.d. | 20.53 | 75.61 | — | — | — |
| FORM 129 | rNLP_S16 | 426.7 | 0.428 | n.d. | 11.69 | 86.11 | — | — | — |
| FORM 134 | rNLP_S17 | 284.3 | 0.135 | 3.72 | 34.47 | 63.46 | — | — | — |
| FORM 135 | rNLP_S18 | 354.1 | 0.091 | 37.4 | 34.63 | 63.76 | — | — | — |
| FORM 136 | rNLP_S19 | 287.7 | 0.06 | 5.86 | 34.47 | 63.46 | — | — | — |
| FORM 137 | rNLP_S20 | 301.6 | 0.091 | 40 | 34.63 | 63.76 | — | — | — |
| FORM 138 | rNLP_S21 | 170.5 | 0.314 | n.d. | 38.5 | — | 50 | — | — |

| | | | | PEG5K | PEG2K | PEG2K | SF |
|---|---|---|---|---|---|---|---|
| Formulation | ID | DOPE | DSPC | PE 14:0 | PE 14:0 | PE 18:0 | Lecithin |
| FORM114 | rNLP_S1 | 10 | — | 1 | — | — | 9 |
| FORM 115 | rNLP_S2 | 25.19 | — | — | — | — | 17.97 |
| FORM 116 | rNLP_S3 | 10 | 9 | 1 | — | — | — |
| FORM 117 | rNLP_S4 | 30.24 | — | — | 1.52 | — | — |
| FORM 118 | rNLP_S5 | 25.19 | — | — | — | — | 17.97 |
| FORM 119 | rNLP_S6 | 25.38 | 17.37 | — | — | — | — |
| FORM 120 | rNLP_S7 | — | 10 | — | — | 1.5 | — |
| FORM 121 | rNLP_S8 | 2.15 | 2.12 | 0.43 | — | — | — |
| FORM 122 | rNLP_S9 | — | 10 | — | — | 1.5 | — |
| FORM 123 | rNLP_S10 | 4.78 | 4.5 | 0.62 | — | — | — |
| FORM 124 | rNLP_S11 | — | — | — | — | — | — |
| FORM 125 | rNLP_S12 | 19.1 | — | 0.83 | — | — | 19.98 |
| FORM 126 | rNLP_S13 | 0.97 | 0.96 | 1.98 | — | — | — |
| FORM 127 | rNLP_S14 | 0.56 | 0.55 | 1.13 | — | — | — |
| FORM 128 | rNLP_S15 | 0.96 | 0.94 | 1.95 | — | — | — |
| FORM 129 | rNLP_S16 | 0.55 | 0.54 | 1.11 | — | — | — |
| FORM 134 | rNLP_S17 | 0.81 | 0.79 | 0.47 | — | — | — |
| FORM 135 | rNLP_S18 | 0.81 | 0.8 | — | — | — | — |
| FORM 136 | rNLP_S19 | 0.81 | 0.79 | 0.47 | — | — | — |
| FORM 137 | rNLP_S20 | 0.81 | 0.8 | — | — | — | — |
| FORM 138 | rNLP_S21 | — | 10 | — | — | 1.5 | — |

TABLE 7
Cargo in the third batch of rNLPs

| | | GFP RNA | SiRNA-GFP | Encapsulation |
|---|---|---|---|---|
| Formulation | ID | (ug/ml) | (ug/mL) | efficiency (%) |
| FORM 114 | rNLP_S1 | 20 | — | 75.27 |
| FORM 115 | rNLP_S2 | 0 | — | n/a |
| FORM 116 | rNLP_S3 | 20 | — | 74.55 |
| FORM 117 | rNLP_S4 | 20 | — | 70.44 |
| FORM 118 | rNLP_S5 | 0 | — | n/a¹ |
| FORM 119 | rNLP_S6 | 20 | — | 67.55 |
| FORM 120 | rNLP_S7 | 20 | — | 80.39 |
| FORM 121 | rNLP_S8 | 20 | — | 88.47 |
| FORM 122 | rNLP_S9 | 20 | — | 86.9 |
| FORM 123 | rNLP_S10 | 20 | — | 88.03 |
| FORM 124 | rNLP_S11 | 20 | — | 92.63 |
| FORM 125 | rNLP_S12 | 20 | — | 91.35 |
| FORM 126 | rNLP_S13 | 50 | — | 82.16 |
| FORM 127 | rNLP_S14 | 50 | — | 81.34 |
| FORM 128 | rNLP_S15 | 50 | — | 96.51 |
| FORM 129 | rNLP_S16 | 50 | — | 96.5 |
| FORM 134 | rNLP_S17 | — | 50 | 63.78 |
| FORM 135 | rNLP_S18 | — | 50 | 99.03 |
| FORM 136 | rNLP_S19 | — | 40 | 98.47 |
| FORM 137 | rNLP_S20 | — | 40 | 98.73 |
| FORM 138 | rNLP_S21 | 68 | — | 95.7 |

¹n/a, not applicable

### Example 2. Composition of NLPs Comprising Pectin

In this example, a novel lipid nanoparticle formulation comprising pectins (PEC) is described for efficient nucleic acid delivery to plants.

### Experimental Procedure

NLPs comprising pectins contain a core of nucleic acids (e.g., siRNA or mRNA) and a lipid shell. Instead of using conventional pegylated lipids, hydrophobic pectin is used. Hydrophobic pectin (either naturally sourced or chemically synthesized) are incorporated in the NLP particles, using any of the production methods described in Example 4, e.g. employing a microfluidics platform, producing the exemplary NLPs as described in Table 8.

TABLE 8
Exemplary compositions (molar percentages) comprising pectin (PEC).

| | | Cationic | Ionizable | | | Polynucleotide |
|---|---|---|---|---|---|---|
| ID | Sterol¹ | lipid² | lipid³ | Phospholipid⁴ | PEC⁵ | (ug/ml)⁶ |
| rNLP_PEC1 | 30 | 20 | — | 40 | 10 | 1 |
| rNLP_PEC2 | 30 | — | 20 | 40 | 10 | 1 |

¹E.g. one or more of cholesterol and/or sistosterol
²E.g. one or more of DDAB, DOTAP, MC3, KC2, and DODMA
³E.g. one or more of SM-102, ALC-315, and DODAP
⁴E.g. one or more of DOPE, sunflower lecithin (de-oiled), soybean lecithin (de-oiled)
⁵E.g. a hydrophobically modified pectin derivative
⁶E.g. an mRNA or an siRNA

### Example 3: Composition of NLP Comprising Boron-Containing Lipids

This Example describes the design and optimization of a new class of modified lipids for efficient nucleic acid delivery to plants.

### Experimental Procedure

Boronic acid lipids are incorporated in the NLP particles, using any of the production methods described in Example 4, e.g. using a microfluidics platform, producing the exemplary NLPs as described in Table 9.

TABLE 9
Exemplary compositions (molar percentages) comprising boronic acid lipids (BOR).

| | | Cationic | Ionizable | | | | Polynucleotide |
|---|---|---|---|---|---|---|---|
| ID | Sterol¹ | lipid² | lipid³ | Phospholipid⁴ | BOR⁵ | PEG⁶ | (ug/ml)⁷ |
| rNLP_BOR1 | 30 | 20 | — | 40 | 10 | — | 1 |
| rNLP_BOR2 | 30 | — | 20 | 40 | 10 | — | 1 |
| rNLP_BOR1 | 30 | 20 | — | 40 | 8 | 2 | 1 |
| rNLP_BOR2 | 30 | — | 20 | 40 | 8 | 2 | 1 |

¹E.g. one or more of cholesterol and sistosterol
²E.g. one or more of DDAB, DOTAP, MC3, KC2, and DODMA
³E.g. one or more of SM-102, ALC-315, and DODAP
⁴E.g. one or more of DOPE, sunflower lecithin (de-oiled), and soybean lecithin (de-oiled)
⁵E.g. a boronic acid lipid.
⁶E.g. PEG5K PE 14:0, PEG2K PE 14:0, or PEG2K PE 18:0
⁷E.g. an mRNA or an siRNA; concentration refers to ug per ml of rNLP suspension

### Example 4. Methods of Making NLPs

This Example describes methods of producing NLPs comprising a hydrophylic core as described herein.

### Experimental Procedure

Several methods have been employed to generate the NLPs described herein. In all methods, an organic phase comprising one or more of phospholipids, sterols, synthetic lipids, and pegylated lipids, is mixed with an aqueous phase comprising de-ionized water and a hydrophilic heterologous functional agent, e.g. a polynucleotide, applying a source of energy to facilitate the formation of NLPs.

The first method employed a hand-pipetting method of mixing a lipid solution (ethanolic) and nucleic acid solution (aqueous) drop by drop, wherein a consistent ratio of lipid-to-nucleic acid was maintained during pipetting while vortexing the mixture. Other methods employed were using microfluidic platforms for nanoparticle synthesis, e.g. the NanoAssemblr® platform or employing e.g. the Sunshine platform from Unchained Labs. Exemplary methods of nanoparticle formation are reviewed in John et al., Pharmaceutics 16(1): 131 (2024), Niculescu et al., Int. J. Mol Sci 23(15): 8293 (2022) and in Petersen et al., Eur. J. Pharmaceut. Biopharmaceut. 192: 126-135 (2023), Other methods of nanoparticle formation are well known in the art.

Particle size, polydispersity index (PDI) and zeta potential were measured using a Malvern Panalytical Zetasizer Ultra Red Label, immediately after production.

The polynucleotide concentration was measured (post-NA, final) and Quant-it™ RiboGreen RNA Assay Kit was used to calculate the loading efficiency. Using the Quant-iT™ RiboGreen™ RNA method, RNA quantification is achieved by mixing the RNA sample with the RiboGreen™ reagent, which binds selectively to RNA. Upon binding, the complex emits a strong fluorescent signal measured by a fluorometer. The fluorescence intensity is directly proportional to the RNA concentration, allowing precise quantification by comparing the sample's fluorescence to a standard curve generated with known RNA concentrations. The encapsulation efficiency was calculated by the (initial concentration of polynucleotide added/actual concentration of polynucleotide in the NLP)*100%, immediately after production.

### Example 5. NLP Compositions Comprising Two or More Heterologous Functional Agents

This Example describes NLPs comprising more than one heterologous functional agent, e.g., a polynucleotide (e.g. mRNA) and a nuclease inhibitor (e.g. Evans blue).

### Experimental Procedure

NLPs comprising more than one heterologous functional agent are produced by any of the methods described in Example 4, e.g. employing a microfluidics device.

An organic phase comprising any of the indicated phospholipid, cholesterol, synthetic lipid and pegylated lipids shown in Tables 2-7 are mixed with an aqueous phase comprising de-ionized water and one or more hydrophilic function agents. In some embodiments, three, four or five agents are combined in one NLP. In some embodiments the NLP comprises a polynucleotide and a polypeptide. In some embodiments, the NLP comprises a polynucleotide (e.g. an mRNA) and a nuclease inhibitor (e.g. Evans blue).

The molar percentages of the phospholipid, cholesterol, synthetic lipid and pegylated lipids can be any of the percentages described in the NLP formulations described in Example 1. The concentrations of the heterologous functional agents in the NLPs can range between 1-10,000 μg of the agent per ml of NLP suspension. Table 10 shows exemplary combinations of two agents, and their final concentrations in the NLPs.

TABLE 10
Production of NLPs comprising more than
one heterologous functional agent

| | | Concen- | | Concen- |
|---|---|---|---|---|
| | | tration | Nuclease | tration |
| Combination^{a} | Polynucleotide | (μg/ml)^{b} | inhibitor | (μg/ml) |
| 1. rNLP(PN + NI) | GPF-mRNA | 20 | Evans blue | 1 |
| 2. rNLP(PN + NI) | GPF-mRNA | 40 | Evans blue | 1 |
| 3. rNLP(PN + NI) | GPF-mRNA | 60 | Evans blue | 1 |
| 4. rNLP(PN + NI) | GPF-mRNA | 40 | Evans blue | 1 |
| 5. rNLP(PN + NI) | GPF-mRNA | 40 | Evans blue | 2 |
| 6. rNLP(PN + NI) | GPF-mRNA | 40 | Evans blue | 3 |

^{a}PN, polynucleotide; NI, nuclease inhibitor
^{b}Concentration refers to ug GPF-mRNA comprised per ml of rNLP suspension

### Example 6. NLP Compositions Each Comprising a Different Heterologous Functional Agent

This Example describes methods to produce mixtures of NLPs populations, e.g. a mixture of a population of NLPs comprising a first heterologous functional agent (e.g. CAS-mRNA) and a second population of NLPs comprising a second heterologous functional agent (e.g. gRNA).

### Experimental Procedure

To produce the two populations of NLPs each comprising a different heterologous functional agent, any of the methods described in Example 4 can be used, e.g. employing a nanofluidics platform.

To produce a NLP mixture, a first population of NLPs comprising a first heterologous functional agent, e.g. a first polynucleotide, is mixed with a second population of NLPs, comprising a second heterologous functional agent, e.g. a second polynucleotide. The first and the second population of NLPs can be mixed, e.g. in equal volumes, to produce the NLP mixture (Table 11).

The molar percentages of e.g. phospholipid, cholesterol, synthetic lipid and pegylated lipids in the formulations can be any of the percentages described in Example 1. The concentrations of the heterologous functional agents comprised in any of the NLP formulations can range between 1-10,000 pg per ml of suspension. The ratios in which the two NLP populations can be mixed is 50:50, or any other ratio, e.g. 10:90.

TABLE 11
Exemplary combinations of two NLP populations, each
comprising a different heterologous functional agent.

| | | Concen- | | Concen- |
|---|---|---|---|---|
| | First | tration | Second | tration |
| Combination^{a} | polynucleotide | (μg/ml)^{b} | polynucleotide | (μg/ml)^{b} |
| 1. rNLP1 (PN1) + | CAS-mRNA | 20 | gRNA | 40 |
| rNLP2 (PN2) | | | | |
| 2. rNLP1 (PN1) + | CAS-mRNA | 40 | gRNA | 40 |
| rNLP2 (PN2) | | | | |
| 3. rNLP1 (PN1) + | CAS-mRNA | 60 | gRNA | 40 |
| rNLP2 (PN2) | | | | |
| 4. rNLP1 (PN1) + | CAS-mRNA | 40 | gRNA | 20 |
| rNLP2 (PN2) | | | | |
| 5. rNLP1 (PN1) + | CAS-mRNA | 40 | gRNA | 40 |
| rNLP2 (PN2) | | | | |
| 6. rNLP1 (PN1) + | CAS-mRNA | 40 | gRNA | 60 |
| rNLP2 (PN2) | | | | |

^{a}PN1, first polynucleotide; PN2, second polynucleotide
^{b}Concentration refers to ug PN1 or PN2 comprised per ml of rNLP suspension

### Example 7. a Composition Comprising NLPs and an Unencapsulated Agent

This Example describes methods to produce a mixture comprising an NLPs population comprising a first heterologous agent (e.g. a polynucleotide), and an unencapsulated second heterologous functional agent (e.g. rapamycin).

### Experimental Procedure

To produce NLPs comprising a first heterologous functional agent, any of the NLP production methods of Example 4 is used, e.g. a method employing a microfluidics device.

The NLPs composition produced is then mixed with an unencapsulated heterologous functional agent, e.g. a regulator of the TOR signaling pathway, e.g. rapamycin (Tee and Faulkner, New Phytologist 243 (1), 32-47 (2024)). Exemplary compositions that can be produced are shown in Table 12.

TABLE 12
Production of a mixture comprising a plurality
of NLPs and an unencapsulated agent

| | | Concen- | Agent that | Concen- |
|---|---|---|---|---|
| | Poly- | tration | opens the | tration |
| Combination^{a} | nucleotide | (μg/ml)^{b} | plasmodesmata | (mM)^{c} |
| 1. rNLP(PN) + | mRNA | 20 | rapamycin | 10 |
| agent | | | | |
| 2. rNLP(PN) + | mRNA | 40 | rapamycin | 10 |
| agent | | | | |
| 3. rNLP(PN) + | mRNA | 60 | rapamycin | 10 |
| agent | | | | |
| 4. rNLP(PN) + | mRNA | 40 | rapamycin | 5 |
| agent | | | | |
| 5. rNLP(PN) + | mRNA | 40 | rapamycin | 10 |
| agent | | | | |
| 6. rNLP(PN) + | mRNA | 40 | rapamycin | 20 |
| agent | | | | |

^{a}PN, polynucleotide
^{b}Concentration refers to ug mRNA comprised per ml of rNLP suspension
^{c}Concentration refers to mmol rapamycin per 1 of rNLP suspension

### Example 8. Loading NLPs with Cargo after NLP Formation

This example describes methods of loading NLPs with additional small molecules, proteins, and nucleic acids to use as probes to determine NLP uptake efficiency in plants.

### Experimental Procedure

a) Loading Additional Small Molecules into NLPs

NLPs are produced by any of the methods described in Example 4. To load e.g. small molecules into NLPs, NLPs are placed in PBS solution with the small molecule either in solid form or solubilized. The solution is left for 1 hour at 22° C., according to the protocol in Sun, Mol. Ther., 2010. Alternatively, the solution is sonicated to induce poration and diffusion into the NLPs according to the protocol from Wang et al, Nature Comm., 4: Article number: 1867, 2013. Alternatively, NLPs are electroporated according to the protocol from Wahlgren et al, Nucl. Acids. Res., 40(17): e130, 2012. Before use, the loaded NLPs are purified to remove unbound small molecules.

b) Loading Proteins or Peptides into NLPs

NLPs are produced by any of the methods described in Example 4. To load additional proteins or peptides into NLPs, NLPs are placed in solution with the protein or peptide in PBS. If the protein or peptide is insoluble, pH is adjusted until it is soluble. If the protein or peptide is still insoluble, the insoluble protein or peptide is used. The solution is then sonicated to induce poration and diffusion into the NLPs according to the protocol from Wang et al, Nature Comm., 4: Article number: 1867, 2013. Alternatively, NLPs are electroporated according to the protocol from Wahlgren et al, Nucl. Acids. Res., 40(17): e130, 2012. Before use, the loaded NLPs are purified to remove unbound peptides and protein. To measure loading of the protein or peptide, the Pierce Quantitative Colorimetric Peptide Assay is used on a small sample of the loaded and unloaded NLPs.

c) Loading Nucleic Acids into NLPs after NLP Formation

NLPs are produced by any of the methods described in Example 4. To load nucleic acids into NLPs, NLPs are placed in solution with the nucleic acid in PBS. The solution is then sonicated to induce poration and diffusion into the NLPs according to the protocol from Wang et al, Nature Comm., 4: Article number: 1867, 2013. Alternatively, NLPs are electroporated according to the protocol from Wahlgren et al, Nucl. Acids. Res., 40(17): e130, 2012. Before use, the NLPs are purified to remove unbound nucleic acids. Nucleic acids that are loaded in the NLPs are quantified using e.g. the Quant-It assay from Thermo Fisher following manufacturer's instructions, or fluorescence is quantified with a plate reader if the nucleic acids are fluorescently labeled.

### Example 9. Lyophilization and Storage of NLPs

This Example demonstrates that NLP can be freeze-dried and stored at ambient or higher temperature for an extended time.

### Experimental Procedure

An aliquot of 1 ml of NLP formulation is mixed with 1 ml of 20% lyoprotectant comprising maltodextrin 12, trehalose, sucrose, or mannitol in DI water. After freezing at −80° C. the suspensions are lyophilized in a LABCONCO FreeZone −84C Benchtop Freeze Dryer. The method is performed with the collector at −100° C. under vacuum to 0.000 mbar. The primary drying step is run for 10 hours with the shelf temperature set to −20° C. The secondary drying step is run for 4 hours with the shelf temperature set to 20° C. Dried NLP suspensions are stored at room temperature. Aliquots of dried NLPs are resuspended in 1 ml of DI water. Stability is assessed by measuring concentrations of the encapsulated cargo over time, and comparison to concentrations used for encapsulation.

Results: Reconstituted NLP's are intact. Dried NLPs have an anticipated higher shelf life than NLPs in suspension, and this method shows that lyophilization and reconstitution does not negatively impact on the structure of the NLPs.

### Example 10. Characterization of NLP Stability and Retention of Encapsulated Bioactive

This example describes measuring the stability of NLPs under a wide variety of storage and physiological conditions.

### Experimental Procedure

NLPs produced as described in Example 4 and are subjected to various conditions. NLPs are suspended in water, 5% sucrose, or PBS and left for 1, 7, 30, and 180 days at −20° C., 4° C., 20° C., and 37° C. NLPs are also suspended in water and dried using a rotary evaporator system and left for 1, 7, and 30, and 180 days at 4° C., 20° C., and 37° C. NLPs are also suspended in water or 5% sucrose solution, flash-frozen in liquid nitrogen and lyophilized. After 1, 7, 30, and 180 days, dried and lyophilized NLPs are then resuspended in water. The previous three experiments with conditions at temperatures above 0° C. are also exposed to an artificial sunlight simulator to determine content stability in simulated outdoor UV conditions. NLPs are also subjected to temperatures of 37° C., 40° C., 45° C., 50° C., and 55° C. for 1, 6, and 24 hours in buffered solutions with a pH of 1, 3, 5, 7, and 9 with or without the addition of 1 unit of trypsin or in other simulated gastric fluids.

After each of these treatments, NLPs are brought back to 20° C., neutralized to pH 7.4, and characterized for e.g. particle size, zeta potential and RNA content, using some or all of the methods described in Example 4.

Results: Compositions and conditions are identified conducive for experimental application of the NLP preparations in field trials.

### Example 11. Uptake of rNLP by Arabidopsis thaliana Roots

This example describes the root uptake of various rNLP lipid compositions as described herein in Arabidopsis thaliana seedlings following exposure of the roots of seedlings to rNLP formulations comprising the mRNA of GFP or the mRNA of GFP-NLS.

### Experimental Procedure

### a) Seed Sterilization and Plating

Arabidopsis thaliana seeds were surface sterilized using bleach solution (30% commercial bleach) for 5 min, washed five times with sterile water, and placed at 4° C. for 48 hours prior to plating to allow vernalization. Seeds were plated in squared petri dishes onto Murashige and Skoog (MS) salt mixture diluted 1:1 with water (0.5×MS) including vitamins and without sucrose (Duchefa, Cat. #NM0222) and 0.8% plant agar (Duchefa at. N #P1001) and grown for six days.

b) Treatments with rNLP

Six-day-old seedlings are transferred to a 24 multi-well plate containing 400 μl of the treatment solution in 0.5×MS. The treatment solutions were one of the following: 1) Negative control of only 0.5×MS media; 2) Negative control of an empty rNLP; 3) Negative control of unencapsulated RNA; or 4) Positive samples of rNLP containing mRNA of GFP or GFP-NLS Treatment lasted for 6 to up to 48 hours (as indicated the Figure) and fluorescence was visualized under a confocal microscope (FV 1000 Olympus confocal microscope, Zeiss Elyra 980 confocal microscope) using the 40× objective and selecting Excitation and Emission wavelengths of 488 nm and 510 nm, respectively (Em/Ex 488/510).

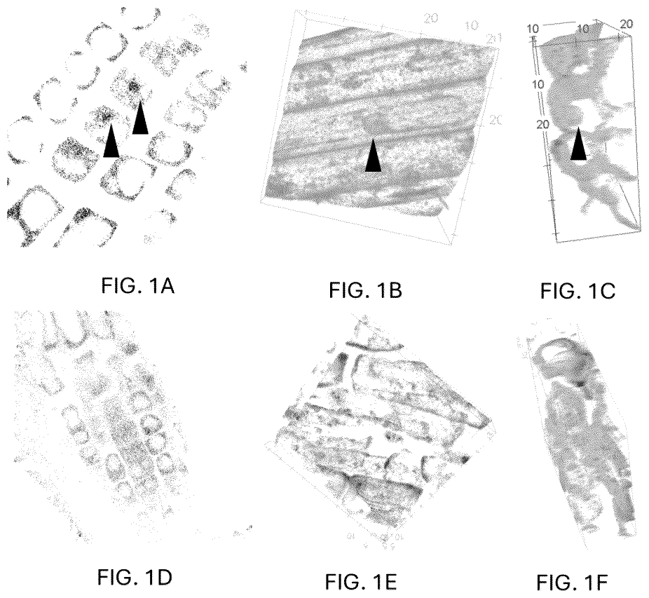

Results: FIG. 1A-1F show a clear differential visualization of GFP signal through confocal microscopy in cell cytoplasm of the root epidermal or cortex cells, upon treatment with rNLPs comprising mRNA of GFP, or in the cell nuclei of those cells, upon treatment with rNLPs comprising mRNA of GFP-NLS (FORM 120). The negative control FORM030 did not show a signal in the same regions (FIG. 1D-1F). The number of experimental repeats and number of root cells analyzed are summarized in Table 13, showing differential results depending on the NLP composition.

TABLE 13
Number of experimental repeats and Arabidopsis root cells analyzed

| | | GFP RNA | | | | # |
|---|---|---|---|---|---|---|
| | | (ug/ml; | # | # Roots | # Cells | Positive |
| Formulation | ID | actual)^{a} | Replicates | observed | observed | cells |
| FORM138 | rNLP_S21 | 65.076 | 1 | 4 | 80 | 1 |
| FORM126 | rNLP_S13 | 41.08 | 2 | 8 | 160 | 3 |
| FORM078 | rNLP_I18 | 18.508 | 9 | 23 | 460 | 0 |
| FORM079 | rNLP_I19 | 19.076 | 9 | 12 | 240 | 0 |
| FORM120 | rNLP_S7 | 16.078 | 3 | 12 | 240 | 5 |
| FORM065 | rNLP_I13 | 18.728 | 3 | 25 | 500 | 5 |
| FORM029 | rNLP_H3 | 18.614 | 6 | 42 | 840 | 30 |
| FORM030 | rNLP_H4 | 0 | 17 | 57 | 1140 | 0 |
| (Neg. Ctrl) | | | | | | |
| FORM058 | rNLP_I6 | 18.154 | 1 | 4 | 80 | 0 |
| FORM059 | rNLP_I7 | 18.258 | 1 | 5 | 100 | 0 |
| FORM064 | rNLP_I12 | 16.436 | 1 | 13 | 260 | 0 |
| FORM050 | rNLP_H12 | 18.106 | 1 | 6 | 120 | 0 |
| FORM054 | rNLP_I2 | 18.584 | 3 | 25 | 500 | 0 |
| FORM055 | rNLP_I3 | 18.544 | 3 | 21 | 420 | 0 |
| FORM121 | rNLP_S8 | 17.694 | 3 | 8 | 160 | 0 |
| FORM123 | rNLP_S10 | 17.606 | 2 | 6 | 120 | 0 |
| FORM128 | rNLP_S15 | 48.255 | 2 | 8 | 160 | 0 |
| FORM129 | rNLP_S16 | 48.25 | 2 | 8 | 160 | 0 |
| FORM127 | rNLP_S14 | 40.67 | 2 | 8 | 160 | 0 |
| FORM130 | rNLP_I26 | 19.56 | 1 | 10 | 200 | 0 |
| FORM131 | rNLP_I27 | 8.578 | 1 | 10 | 200 | 0 |

^{a}Initial loading concentration multiplied by the encapsulation efficiency, as determined immediately after production. Concentration refers to ug GFP RNA per ml of rNLP suspension.

### Example 12. Uptake of rNLP into Arabidopsis thaliana Leaves

This example describes the results of experiments examining the uptake of rNLP formulations encapsulating mRNA of GFP-NLS in Arabidopsis thaliana shoots and the subcellular localization of t rNLP compositions in Arabidopsis thaliana leaves.

### Experimental Procedure

### a) Seed Sterilization and Plating

Arabidopsis seeds were surface sterilized with bleach solution (30% commercial bleach) for 5 minutes, washed 5 times with sterile water, and placed at 4° C. for 48 hours prior to plating. Seeds were plated in squared petri dishes onto 0.5× strength Murashige and Skoog salt mixture (0.5×MS) including vitamins and without sucrose (Duchefa, Cat. n #M0222) and 0.8% plant agar (Duchefa Cat. n #P1001) and grown for twelve days in long day at 22° C.

b) rNLP Treatments

Twelve-day-old seedlings were transferred to 24-well plates containing 400 μl of the treatment solution. Incubations carried out for 6 out to 24 hours. Treatment solutions were one of the following: 1) 0.5×MS media; 2) Unencapsulated mRNA of GFP-NLS sequence; 3) An empty rNLP formulation; 4) A formulation containing mRNA of GFP-NLS sequence.

After 6 to 24h of incubation, true leaves of the Arabidopsis rosette were cut and placed in microscopy slides to be imaged in their abaxial side. Leaves were visualized by confocal microscopy using a Zeiss Elyra 980 microscope using GFP settings (Excitation 488 nm, emission 509 nm). Epidermal pavement cells were observed.

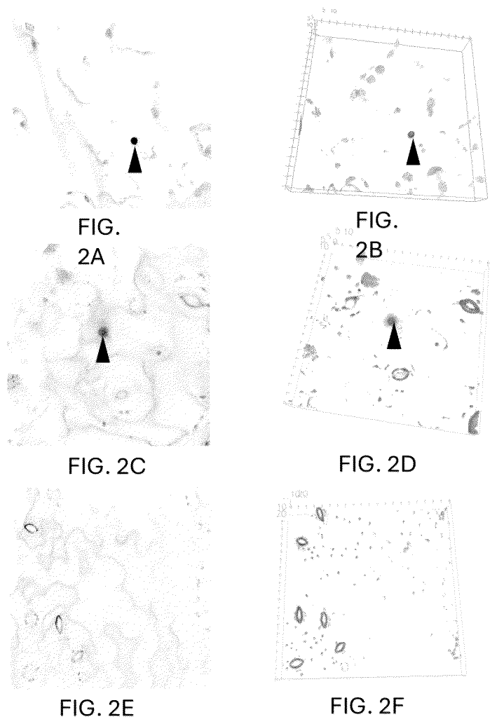

Results: As shown in Table 14(a), of the 61 rNLP formulations tested, uptake and GLP-NLS expression was observed for Form 65, Form 78, Form 79, Form 134 and Form138. FIG. 2A-2F depict the visualization, using z-stack of the epidermis in the abaxial side, of nuclear GFP signal in pavement cells of leaves treated with rNLP formulations containing mRNA of GFP-NLS (FORM138). In contrast, nuclear signal in negative controls carried out with FORM138 not comprising RNA (“neg”) were non-existent. The number of experimental repeats and cells analyzed are shown in Table 14(b), showing differential results depending on the NLP composition.

TABLE 14(a)
Detection of GLP-NLS expression in treated Arabidopsis shoots.

| Type | Form | GFP-NLS Detected |
|---|---|---|
| rNLP_H1 | 27 | No |
| rNLP_H2 | 34 | No |
| rNLP_H3 | 29 | No |
| rNLP_H4 | 30 | No |
| rNLP_H5 | 32 | No |
| rNLP_H6 | 33 | No |
| rNLP_H7 | 31 | No |
| rNLP_H8 | 45 | No |
| rNLP_H9 | 46 | No |
| rNLP_H10 | 48 | No |
| rNLP_H11 | 49 | No |
| rNLP_H12 | 50 | No |
| rNLP_I1 | 47 | No |
| rNLP_I2 | 54 | No |
| rNLP_I3 | 55 | No |
| rNLP_I4 | 56 | No |
| rNLP_I5 | 57 | No |
| rNLP_I6 | 58 | No |
| rNLP_I7 | 59 | No |
| rNLP_I8 | 60 | No |
| rNLP_I9 | 61 | No |
| rNLP_I10 | 62 | No |
| rNLP_I11 | 63 | No |
| rNLP_I12 | 64 | No |
| rNLP_I13 | 65 | Yes |
| rNLP_I14 | 66 | No |
| rNLP_I15 | 74 | No |
| rNLP_I16 | 75 | No |
| rNLP_I17 | 76 | No |
| rNLP_I18 | 78 | Yes |
| rNLP_I19 | 79 | Yes |
| rNLP_I20 | 83 | No |
| rNLP_I21 | 84 | No |
| rNLP_I22 | 85 | No |
| rNLP_I23 | 86 | No |
| rNLP_I25 | 113 | No |
| rNLP_I26 | 130 | No |
| rNLP_I27 | 131 | No |
| rNLP_I28 | 132 | No |
| rNLP_I29 | 133 | No |
| rNLP_S1 | 114 | No |
| rNLP_S2 | 115 | No |
| rNLP_S3 | 116 | No |
| rNLP_S4 | 117 | No |
| rNLP_S5 | 118 | No |
| rNLP_S6 | 119 | No |
| rNLP_S7 | 120 | No |
| rNLP_S8 | 121 | No |
| rNLP_S9 | 122 | No |
| rNLP_S10 | 123 | No |
| rNLP_S11 | 124 | No |
| rNLP_S12 | 125 | No |
| rNLP_S13 | 126 | No |
| rNLP_S14 | 127 | No |
| rNLP_S15 | 128 | No |
| rNLP_S16 | 129 | No |
| rNLP_S17 | 134 | Yes |
| rNLP_S18 | 135 | No |
| rNLP_S19 | 136 | No |
| rNLP_S20 | 137 | No |
| rNLP_S21 | 138 | Yes |

TABLE 14(b)
Number of experimental repeats and Arabidopsis leaf cells analyzed.

| | | GFP-RNA | | | | |
|---|---|---|---|---|---|---|
| | | (ug/ml; | # | # Shoots | # Cells | # Positive |
| Formulation | ID | actual)^{a} | Replicates | observed | observed | cells |
| FORM138 | rNLP_S21 | 65.076 | 5 | 18 | 360 | 27 |
| FORM138-1 neg^{b} | rNLP_S21-1 | 0 | 4 | 5 | 140 | 0 |
| | neg^{b} | | | | | |
| FORM078 | rNLP_I18 | 18.508 | 3 | 3 | 90 | 4 |
| FORM079 | rNLP_I19 | 19.076 | 3 | 6 | 180 | 3 |
| FORM065 | rNLP_I13 | 18.728 | 2 | 8 | 143 | 7 |
| FORM134 | rNLP_S17 | 12.756 | 2 | 8 | 55 | 3 |

^{a}Initial loading concentration multiplied by the encapsulation efficiency, as determined immediately after production. Concentration refers to ug GFP RNA per ml of rNLP suspension.
^{b}Second batch, produced without RNA (“neg”).

### Example 13. Uptake and Transport of rNLP to Different Aerial Organs of Arabidopsis thaliana Assessed with NIA2, PDS3 and FT amiRNA

This Example describes the transport of a selection of rNLP compositions across the organs of Arabidopsis thaliana. following the exposition of organs to an rNLP formulation comprised of an amiRNA, e.g. targeting NIA2, PDS3 and FT, capable of causing a phenotypical effect in the plant. For example, an amiRNA sequence against FT signaling in Arabidopsis will cause late flowering.

### Experimental Procedure

### a) Seed Sterilization and Growth

Arabidopsis seeds are surface sterilized with bleach solution (30% commercial bleach) for 5 minutes, washed 5 times with sterile water, and placed at 4 C for 48 hours prior to plating. Seeds are plated in squared petri dishes onto 0.5× Murashige and Skoog (MS) salt mixture including vitamins and without sucrose (Duchefa, Cat. n #M0222) and 0.8% plant agar (Duchefa Cat. n #P1001) and grown for six days in the darkness at 22° C.

b) Treatment with rNLPs Comprising amiRNA

### 1) Targeting NIA2

Seedlings are transferred to 70 μm cell strainers (bd falcon) to 6-well plates containing 1 ml of rNLP solution and 1 ml of 0.5×MS including vitamins and without sucrose (Duchefa, Cat. N #M0222) and 0.8% plant agar (Duchefa Cat. n #P1001). Treatment solutions are one of the following: 1) rNLP-NIA2 amiRNA; 2) rNLP-Empty (No RNA); 3) unencapsulated amiRNA; 4) 0.5×MS media. Incubations are conducted for 24 hr and in the dark to avoid dye degradation and seedlings transferred in a new petri dish NH3-deficient media and 0.8% plant agar (Duchefa Cat. n #P1001).

Results: plants treated with rNLPs containing NIA2 amiRNA in the leaf or in the root, and grown in a NH3-deficient media, show chlorosis or whitening in the leaf when compared to WT control plants, measured by chlorophyll quantitation. Plants treated with empty rNLPs or rNLPs containing a mock, inactive RNA sequence, and grown in a NH3-deficient media show a chlorophyll quantitation similar to WT control plants. Transgenic control plants expressing 35S:amiRnia2 in the leaf or in the root, and grown in a NH3-deficient media, show chlorosis or whitening in the leaf when compared to WT control plants.

### 2) Targeting PDS3

Arabidopsis seedlings are grown to 6-day-old and transferred to soil in greenhouse conditions, where they are left to grow up to 21-day-old. The genotypes tested are the following: 1) A WT used for control and experimentation; 2) A positive control overexpressing the amiRNA under the 35S promoter; 3) Treatments with selected rNLP. After 21 days, and daily in subsequent days, the leaves of the Arabidopsis rosette are infiltrated with the following formulations: 1) A negative control of water; 2) An empty NLP formulation as negative control; 3) An unencapsulated amiRNA; 4) A rNLP formulation containing amiRNA against the desired trait such as flowering by targeting PDS3.

Results: Plants treated with rNLPs containing anti-PDS3 amiRNA in the leaf or in the root show chlorosis or whitening in the leaf when compared to WT control plants, measured by chlorophyll quantitation. Plants treated with empty rNLPs or rNLPs containing a mock, inactive RNA sequence show a chlorophyll quantitation similar to WT control plants. Transgenic control plants expressing 35S: pds3 amiRNA in the leaf or in the root, show chlorosis or whitening in the leaf when compared to WT control plants.

### 3) Targeting FT

Arabidopsis seedlings are grown to 6-day-old and transferred to soil in greenhouse conditions, where they are left to grow up to 21-day-old. The genotypes tested are the following: 1) A WT used for control and experimentation; 2) A positive control overexpressing the amiRNA under the 35S promoter; 3) Treatments with selected rNLP. After 21 days, and daily in subsequent days, the leaves of the Arabidopsis rosette are infiltrated with the following formulations: 1) A negative control of water; 2) An empty NLP formulation as negative control; 3) An unencapsulated amiRNA; 4) A rNLP formulation containing amiRNA against the desired trait such as flowering by targeting FT.

Results: Plants treated with rNLPs containing FT amiRNA in the leaf or in the root show a delay in flowering time when compared to WT control plants. Plants treated with empty NLPs or rNLPs containing a mock, inactive RNA sequence show a flowering time similar to WT control plants. Transgenic control plants expressing 35S:amiR-ft amiRNA in the leaf or in the root show a delay in flowering time when compared to WT control plants.

### Example 14. Uptake of rNLPs and Organ Tissue Distribution in Arabidopsis thaliana

This Example describes the transport of a selection of rNLP compositions across the organs of Arabidopsis thaliana.

### Experimental Procedure

### a) Seed Sterilization and Plating

Arabidopsis seeds are surface sterilized with bleach solution (30% commercial bleach) for 5 minutes, washed 5 times with sterile water, and placed at 4° C. for 48 hr prior to plating. Seeds are plated in squared petri dishes onto 0.5× Murashige and Skoog (MS) salt mixture including vitamins and without sucrose (Duchefa, Cat. n #M0222) and 0.8% plant agar (Duchefa Cat. n #P1001) and grown for six days in the darkness at 22° C.

b) Treatments with rNLP

Seedlings are transferred to 70 μm cell strainers (bd falcon) to 6-well plates containing 1 ml of rNLP solution and 1 ml of 0.5×MS including vitamins and without sucrose (Duchefa, Cat. N #M0222) and 0.8% plant agar (Duchefa Cat. n #P1001). Treatment solutions are one of the following: 1) rNLP-RNA; 2) NLP-Empty (No RNA); 3) unencapsulated RNA; or 4) 0.5×MS media.

Incubations are conducted for 24 hr and in the dark to avoid dye degradation. Seedlings are visualized using a confocal microscope (FV 1000 Olympus confocal microscope) using 20× objective and confocal microscope settings of Ex/Em: 488/520 nm. The experiment is performed three times and at least ten independent roots are visualized for each treatment.

Results: Seedlings treated with rNLP-RNA formulation present expression of GFP in roots, hypocotyls and/or cotyledons. Seedlings treated with NLP-Empty, RNA unencapsulated and MS media no present GFP fluorescence.

### Example 15. Uptake of NLPs and Meristem Targeting in Arabidopsis thaliana

This example describes the uptake of rNLP formulations by A. thaliana.

### Experimental Procedure

### a) Seed Sterilization and Plating

Seeds are surface sterilized using bleach solution (30% commercial bleach) for 5 min, washed five times with sterile water, and placed at 4° C. for 48 hr prior to plating to allow vernalization. Seeds are plated in squared petri dishes onto Murashige and Skoog (MS) salt mixture diluted 1:1 with water (0.5×MS) including vitamins and without sucrose (Duchefa, Cat. #NM0222) and 0.8% plant agar (Duchefa at. N #P1001) and grown for six days.

b) Treatments with NLP

Six-day-old seedlings are transferred to a 24 multi-well plates containing 400 μl of the treatment solution in 0.5×MS. The treatment solutions are one of the following: 0.5MS, NLP (empty); NLP-RNA (NLS-GFP or others); unencapsulated RNA. Confocal laser scanning microcopy of GFP expression in planta is performed by using either the FluoView™ FV1000 microscope (Olympus), or Zeiss LSM 980, with 40× objective and confocal microscope settings of Ex/Em: 488/520 nm.

Results: Seedlings treated with select NLP formulations present GFP fluorescence signal in the nuclei of cells localized the shoot apical meristem whereas no fluorescence is detected in seedlings treated with NLP (empty), unencapsulated GFP RNA and MS.

### Example 16. Uptake of NLP by Tomato Root Epidermis Cells

This example describes the uptake of NLP formulations by a crop species.

### Experimental Procedure

### 1) Seed Sterilization and Plating

Tomato seeds are surface sterilized using bleach solution (30% commercial bleach) for 10 minutes min, washed 5 times with sterile water, and placed at 4° C. for 72 h prior to plating to allow vernalization. Seeds are plated in squared petri dishes onto 0.5× Murashige and Skoog (MS) salt mixture including vitamins without sucrose (Duchefa, Cat. #M0222) and 0.8% plant agar (Duchefa Cat. #P1001) and grown for six days at 22° C. in long day conditions.

2) Treatments with NLP

Six-day-old tomato seedlings are transferred to a 24 multi-well plates containing 400 μl of the treatment solution. The treatment solutions are one of the following: MS buffer, NLP (empty); NLP-RNA (NLS-GFP or others); unencapsulated RNA. Incubations are conducted for 24 hours, and fluorescence is visualized using a confocal microscope (FV 1000 Olympus confocal microscope) using the 40× objective with confocal settings of Ex/Em: 480/520 nm. The experiment is performed with 10 replicate seedlings.

Results: Plants treated with select rNLPs containing GFP-NLS RNA presented cells expressing GFP in the nuclei of tomato roots. Neither unencapsulated RNA nor the formulations without RNA show GFP signal in the nuclei.

### Example 17. NLP Formulations with Polymers for Seed Application

This example describes NLPs mixed with polymers (e.g. Kannar SeedKOTE Corn Neutral v2.8) and coating of seeds.

### Experimental Procedure

### a) Preparation of Corn Germination Boxes

Germination boxes are sterilized and Phytagel poured one day in advance (Day-6) and stored covered at 4° C. for one day. Large systema lunchboxes are sterilized by soaking in 10% bleach for ≥20 min. 70% EtOH is sprayed to completely coat the boxes and then let to dry. 500 ml of Phytagel comprising 0.7 g of Phytagel and 0.65 g of Hoaglands' basal Salt No. 2, pH 6.5, is poured into the boxes and allowed to cool.

### b) Sterilization of Corn Seeds

Seeds are placed in a large, open-mouthed container and 15% bleach solution is poured just below the container fill line to soak for 15 min. Seeds are rinsed three times with sterile water and placed on the same day as sterilization.

### c) Seed Coating

An aliquot of 200 μL NLP without polymer (Kannar™ SeedKOTE™ Corn Neutral v2.8, Kannar Earth Science) is added to 4 g of seeds (about 25 seeds) in 50 mL tube in dropwise fashion while vortexing. Seeds are removed and placed on a paper towel to air dry.

### d) Germination of Seeds on Phytagel

Corn seeds are placed into a Conviron reach-in incubator (27C, 50% relative humidity, 16h L:8h D). Seeds are placed in a 4×6 grid pattern following the height×length of the box in which phytagel is poured. Seeds are placed embryo side up (scoop up) and gently pressed into gel. Containers are resealed with aluminum foil. On day −4, after 1 day germination time foil is removed and replaced with clear systema lid and let grow for 4 more days. On Day 0, Phytagel Magenta Boxes are prepared for corn and rootworm placement. Magenta boxes are prepared the day before and stored sealed at 4° C. Sterilized magenta boxes are filled with 25 ml of Phytagel solution, allowed to cool, covered with foil or sterilized magenta cage lids and stored at 4° C. if not used immediately.

Results: Seeds coated with select NLP formulations comprising a polymer are taken up by the seeds.

### Example 18. NLP Corn Seed Uptake and Seed Germination after NLP Treatment

This example describes uptake of NLP by seeds and germination after treatment of seeds with NLPs.

### Experimental Procedure

### a) Pre-Germination Assays and Treatment

B104 corn seeds are incubated in water (control) or NLP suspension (diluted 1:2 in MS 0.5×) in a 24-well plate in a volume of 200 ul. Treatment solutions are one of the following: 1) water only; 2) NLP-RNA (GFP mRNA); and 3) NLP-Empty (no RNA), 4) unencapsulated GFP RNA. Seeds are cross sectioned at 24 h and at 48h and imaging of embryos is conducted using a stereomicroscope Olympus SZX16 (GFP filters).

### b) Germination in NLP Solution

B104 corn seeds are incubated in water (control) or NLP suspension (diluted 1:2 in MS 0.5×) in a 24-well plate in a volume of 200 ul. Treatment solutions are one of the following: 1) water only; 2) NLP-RNA (GFP mRNA); and 3) NLP-Empty (no RNA); 4) unencapsulated GFP RNA. Seeds are cross sectioned at 4 days and maize root at 5 days for examination using a stereomicroscope Olympus SZX16 (RFP filters) with 5× magnification.

c) Imbibition with NLP Suspension for 24 h and Germination in Water for 5 Days:

B104 seeds are incubated in water (control) or in NLP suspension (diluted 1:2 in MS 0.5×) in a 24-well plate in a volume of 200 ul for 24h. Treatment solutions are one of the following: 1) water only; 2) NLP-RNA (GFP mRNA); and 3) NLP-Empty (no RNA). Seeds are subsequently germinated in water for 5 days. Seed cross sections are analyzed using a stereomicroscope Olympus SZX16 (RFP filters) with 5× magnification.

Results: Seeds treated with select NLPs containing GFP RNA present cells expressing GFP. Neither the unencapsulated RNA nor the formulations without RNA show GFP signal in the nuclei.

### Example 19. Root Uptake in Corn

This Example describes the uptake of the NLP containing the NLS-GFP RNA in corn epidermis roots.

### Experimental Procedures

### a) Seed Sterilization

Seeds were placed in a 500 ml beaker containing 1 magnet for stirring. A 250 ml of ethanol 70% was added and seeds incubated by stirring for 2 minutes. The ethanol was removed, and 250 ml of 50% commercial bleach was added with a drop of triton X-20 and stirred for 30 minutes. The bleach solution was removed followed by five washed with autoclaved miliQ water of 10 min each while stirring.

### b) Seed Plating

A round sterile filter paper was placed in a petri dish, followed by the addition of 5 ml of 4% preservative for plant culture media (PPM™) comprising 5-chloro-2-methyl-3(2H)-isothiazolone and 2-methyl-3(2H)-isothiazolone (Plant Cell Technologies, Inc), diluted in sterile MiliQ water. Ten seeds were placed per plate and an extra filter was placed on top. 5 ml of 4% ppm was placed on the top the plate is sealed with micropore. Plates were then wrapped in foil and placed in a 25° C. growth chamber for 10 or 12 days.

### c) NLP Treatments

Treatments of seedlings were performed in six-well-plates. Treatment solutions were 1) NLPs comprising RNA (GFP RNA, NLS-GFP or others); 2) NLPs (Empty no RNA); 3) RNA unencapsulated (GFP RNA, NLS-GFP or others); or 0.5 MS buffer. A 500 ul aliquot of the formulation in 2 ml of 0.5 MS is added in each well. Four seedlings were placed in each well with the lateral roots in contact with the NLP-RNA solution. Plates were incubated for 16-24 hours at 25° C. in the darkness.

### d) Root Uptake Visualization by Using Confocal Microscopy Imaging

Confocal microscopy visualization of the epidermal layer of the corn lateral roots for GFP was performed using a Zeiss 980 Elyra 7; Olympus FV1000) with excitation/emission wavelengths settings of 488 nm/520 nm.

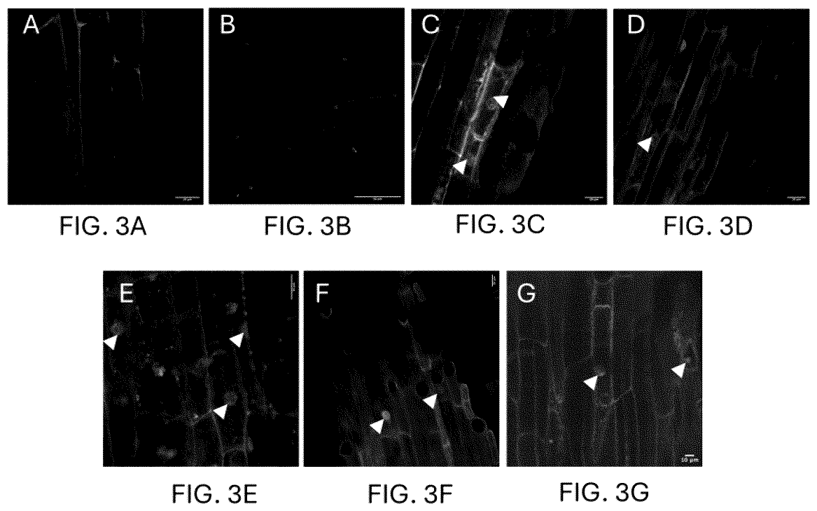

Results: As shown in FIG. 3A-3G, after 16-24 hours of incubation with the indicated rNLP formulations containing GFP-NLS RNA, detection of nuclei expressing GFP was observed in corn root epidermal cells. Neither the unencapsulated RNA of NLS-GFP rNLP nor the formulations without RNA show GFP signal in the nuclei. Table 15(a) summarizes the numbers of repeat experiments and numbers of root epidermal cells analyzed.

TABLE 15(a)
Experimental repeats and corn root cells analyzed

| | | GFP-RNA | | | | # | % cells |
|---|---|---|---|---|---|---|---|
| | | (ug/ml; | | # | # | positive | expressing |
| Formulation | ID | actual)^{a} | #repeats | roots | cells | cells | GFP |
| FORM30 | rNLP_H4 | 0 | 3 | 27 | 206 | 6^{b} | 0.029% |
| (Negative | | | | | | | |
| Control) | | | | | | | |
| FORM78 | rNLP_I18 | 18.508 | 3 | 124 | 491 | 52 | 10.6% |
| FORM79 | rNLP_I19 | 19.076 | 2 | 24 | 495 | 26 | 5.05% |
| FORM128 | rNLP_S15 | 48.255 | 1 | 6 | 108 | 0 | 0 |
| FORM126 | rNLP_S13 | 41.08 | 1 | 6 | 74 | 0 | 0% |
| FORM127 | rNLP_S14 | 40.67 | 1 | 6 | 104 | 0 | 0% |
| FORM126-2^{c} | rNLP_S13-2 | 46.82 | 1 | 7 | 109 | 6 | 5.5% |
| FORM127-2^{c} | rNLP_S14-2 | 47.475 | 1 | 10 | 170 | 4 | 2.3% |
| FORM128 | rNLP_S15 | 48.255 | 1 | 6 | 43 | 0 | 0% |
| FORM129 | rNLP_S16 | 48.25 | 1 | 7 | 73 | 0 | 0% |
| FORM138 | rNLP_S21 | 65.076 | 1 | 15 | 139 | 11 | 7.9% |
| FORM120 | rNLP_S7 | 16.078 | 1 | 15 | 119 | 2 | 1.68% |
| FORM121 | rNLP_S8 | 17.38 | 1 | 12 | 120 | 3 | 2.5% |

^{a}Initial loading concentration multiplied by the encapsulation efficiency, as determined immediately after production. Concentration refers to ug GFP RNA per ml of rNLP suspension.
^{b}one replicate sample contaminated with RNA
^{c}Second batch, produced with a different method.

Similarly, corn shoots were treated with selected rNLP formulations and nuclear GFP expression was assessed. As shown in Table 15(b), nuclearly localized GFP expression was observed in corn shoot cells treated with rNLP formulas 138, 167, 199, 201, 209, 211, 245, 249, 251 and 253.

TABLE 15(b)
Detection of GFP expression in corn
shoot cells treated with rNLPs.

| Type | Form | GFP-NLS Detected |
|---|---|---|
| rNLP_S21 | 138 | Yes |
| rNLP_S22 | 167 | Yes |
| rNLP_S28 | 199 | Yes |
| rNLP_S30 | 201 | Yes |
| rNLP_S32 | 203 | No |
| rNLP_S33 | 209 | Yes |
| rNLP_S35 | 211 | Yes |
| rNLP_S61 | 245 | Yes |
| rNLP_S65 | 249 | Yes |
| rNLP_S67 | 251 | Yes |
| rNLP_S69 | 253 | Yes |

### Example 20. Corn Seed Coating with NLPs and Uptake

This method describes the coating of corn seeds with NLPs and biodistribution of NLPs following corn seed coating.

### Experimental Procedure

### a) Seed Sterilization

Corn seeds are surface sterilized with 100% Ethanol for 3 minutes, subsequently rinsed in a bleach solution (30% commercial bleach 0.01% Tween) for 10 minutes and finally washed ×5 times with sterile water.

### b) NLP Treatment and Plating

An aliquot of 750 ul of NLP 1) water only; 2) NLP-RNA (GFP mRNA); and 3) NLP-Empty (no RNA), or 750 ul of water is added to 25 g of corn seeds in a 50 ml conical tube and shaken with a vortex for 15 seconds to coat the seeds. The coated seeds are emptied onto a flat metal pan and dried under the flow hood overnight. The seeds are then germinated in a petri dish containing wet filter paper and grown at 25° C. in darkness for 5 days.

### c) Imaging

Cross sections of the seeds, roots and shoots are prepared on day 5 for imaging with a stereomicroscope Olympus SZX16 (RFP filters) with 5× magnification in the far-red channel.

Results: Seeds coated with select rNLPs comprising RNA GFP show fluorescence in all plant organs imaged, whereas treatment with unformulated GFP RNA, Empty NLP formulations or water does not produce appreciable fluorescence inside the plant tissues.

### Example 21. Distribution in Corn

This Example describes NLP distribution in corn seedlings.

### Experimental Procedure

### a) Seed Sterilization

Seeds are placed in a 500 ml beaker containing 1 magnet for stirring. A 250 ml of ethanol 70% is added and seeds incubated by stirring for 2 minutes. The ethanol is removed, and 250 ml of 50% commercial bleach is added with a drop of triton X-20 and stirred for 30 minutes. The bleach solution is removed followed by five washed with autoclaved miliQ water of 10 min each while stirring.

### b) Seed Plating

A round sterile filter paper is placed in a petri dish, followed by the addition of 5 ml of 4% PPM™ diluted in sterile MiliQ water. Ten seeds are placed per plate and an extra filter is placed on top. 5 ml of 4% ppm is placed on the top the plate is sealed with micropore. Plates are then wrapped in foil and placed in a 25° C. growth chamber for 10 or 12 days.

### c) NLP Treatments

Treatments are performed in 2 ml Eppendorf tubes. Treatment solutions are 1) NLPs comprising RNA (GFP RNA, NLS-GFP or others); 2) NLPs (Empty no RNA); 3) RNA unencapsulated (GFP RNA, NLS-GFP or others); A 500 ul of the NLP formulation is added in 1 ml of 0.5×MS media. Seedlings are placed in the tube and only the root is in contact with the formulation. The tube rack is placed inside a plastic box and incubated for three days at 25° C.

### d) Visualization in Mesocotyl and Leaves by Using Confocal Microscopy Imaging

Confocal microscopy visualization of the epidermal layer of the mesocotyls and leaf surface for GFP is performed using a Zeiss 980 Elyra 7; Olympus FV1000) with excitation/emission wavelengths settings of 488 nm/520 nm.

Results: After 16-24 hours of incubation with the NLP formulation containing NLS-GFP RNA detection of nuclei expressing GFP in corn epidermis roots, mesocotyl and leaves. Neither the unencapsulated RNA of GFP-NLS NLP nor the formulations without RNA show GFP signal in the nuclei.

### Example 22: Uptake of NLP Containing mRNA by Mechanical Uptake in Nicotiana

This example describes the detection of NLP in planta following mechanical application of NLP into the leaves using leaf infiltration in Nicotiana benthamiana to complement the results obtained by root uptake and transport in Arabidopsis (natural uptake). These experiments study the subcellular localization and dynamics of NLP entering the cell via mechanical means.

### Experiment Design

### a) Leaf Infiltration Assay

Nicotiana benthamiana plants were grown for 3-4 weeks in greenhouse conditions. On day 1, an infiltrate 0.1 ml of NLP solutions was infiltrated into the leaf using a 2.5 ml syringe. Samples applied were: 1) NLP solution containing mRNA GFP; 2) NLP solution containing mRNA GFP-NLS; 3) Water control; 4) NLP solution without RNA (empty control); 5) NLP solution without RNA (empty control) labelled with Rhodamine.

### b) Visualization

On different times (24h, 48h, 3 days, 6 days, 10 days) a square piece of the infiltrated leaf was cut and visualized under the confocal microscope (FV 1000 Olympus confocal microscope or Leica SP5) using a 20× objective with confocal settings of Ex/Em: 488/520.

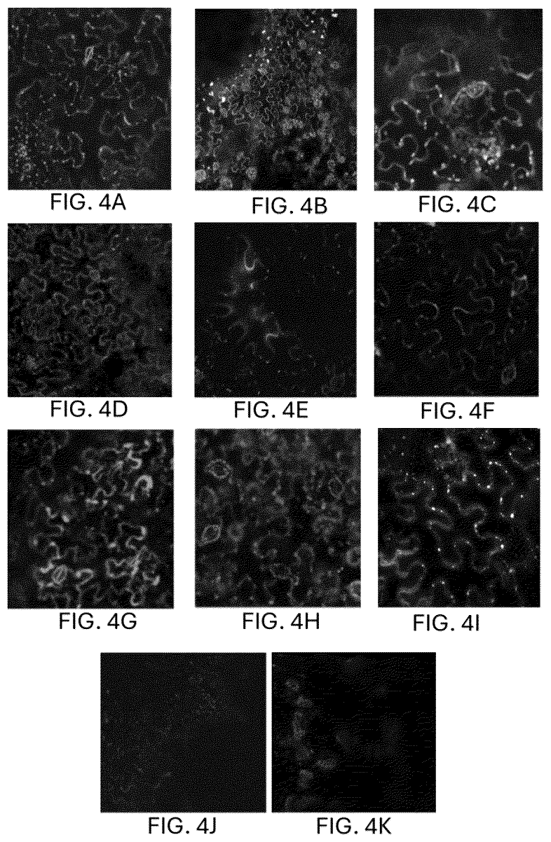

Results: Images obtained are shown in FIG. 4A-4K. On different times (24h, 48h, 3 days, 6 days, 10 days) of incubation with the rNLP formulation containing NLS-GFP RNA, detection of nuclei expressing GFP in the pavement cells was observed. Neither the unencapsulated RNA of GFP-NLS nor the formulations without RNA showed a GFP signal in the nuclei. A summary of GFP detection in Nicotiana leaves treated with rNLP formulations are shown in Table 16.

TABLEs 16
Formulations tested in GFP expression in
the Nicotiana benthamiana leaf assay.

| | | GFP RNA | |
|---|---|---|---|
| Formulation | ID | (ug/ml; actual)^{a} | GFP Detected |
| FORM027 | rNLP_H1 | 16.728 | No |
| FORM029 | rNLP_H3 | 18.614 | Yes |
| FORM030 | rNLP_H4 | 0 (Neg. Crtl) | No |
| FORM049 | rNLP_H11 | 18.96 | No |
| FORM050 | rNLP_H12 | 18.106 | No |
| FORM054 | rNLP_I2 | 18.584 | No |
| FORM055 | rNLP_I3 | 18.544 | No |
| FORM058 | rNLP_I6 | 18.154 | No |
| FORM059 | rNLP_I7 | 18.258 | No |
| FORM060 | rNLP_I8 | 18.434 | No |
| FORM061 | rNLP_I9 | 17.85 | No |
| FORM062-2^{b} | rNLP_I10-1 neg | 0 | No |
| FORM063 | rNLP_I11 | 7.428 | No |
| FORM065 | rNLP_I13 | 18.728 | Yes |
| FORM066-2^{b} | rNLP_I14-2 neg | 0 (Neg. Ctrl.) | No |
| FORM074 | rNLP_I15 | 18.28 | Yes |
| FORM075 | rNLP_I16 | 18.602 | Yes |
| FORM076 | rNLP_I17 | 18.668 | Yes |
| FORM078 | rNLP_I18 | 18.508 | Yes |
| FORM079 | rNLP_I19 | 19.076 | Yes |
| FORM083 | rNLP_I20 | 18.148 | No |
| FORM084 | rNLP_I21 | 18.994 | Yes |
| FORM085 | rNLP_I22 | 19.074 | Yes |
| FORM086 | rNLP_I23 | 17.716 | No |
| FORM113 | rNLP_I25 | 0 | No |
| FORM130 | rNLP_I26 | 19.56 | No |
| FORM131 | rNLP_I27 | 8.578 | No |
| FORM132 | rNLP_I28 | 18.81 | No |
| FORM133 | rNLP_I29 | 10.326 | No |
| FORM114 | rNLP_S1 | 15.05 | No |
| FORM115 | rNLP_S2 | 0 | No |
| FORM116 | rNLP_S3 | 14.91 | No |
| FORM117 | rNLP_S4 | 14.09 | No |
| FORM118 | rNLP_S5 | 0 | No |
| FORM119 | rNLP_S6 | 13.51 | No |
| FORM121 | rNLP_S8 | 16.08 | No |
| FORM122 | rNLP_S9 | 17.69 | No |
| FORM123 | rNLP_S10 | 17.38 | No |
| FORM124 | rNLP_S11 | 17.61 | No |
| FORM125 | rNLP_S12 | 18.53 | No |
| FORM126 | rNLP_S13 | 41.08 | No |
| FORM127 | rNLP_S14 | 40.67 | No |
| FORM128 | rNLP_S15 | 48.26 | No |
| FORM129 | rNLP_S16 | 48.25 | No |
| FORM138 | rNLP_S21 | 65.076 | No |

^{a}Initial loading concentration multiplied by the encapsulation efficiency, as determined immediately after production. Concentration refers to ug GFP RNA per ml of rNLP suspension.
^{b}Second batch, produced without RNA (“neg”).

### Example 23: Uptake of rNLP Containing siRNAs by Mechanical Uptake in Nicotiana

This example describes the use of rNLP containing a siRNA to trigger a functional effect in Nicotiana leaves. Specifically, this Example describes the silencing of GFP genes using rNLPs loaded with siRNAs.

### Experimental Procedure

### a) Materials

The plants employed in this experiment were the Nicotiana benthamiana 16C line (Ruiz et al, Plant Cell 1998, 10 (6) 937-46), and a Nicotiana benthamiana wt. Plants were grown in a growth chamber under a 16 h day: 8 h night photoperiod. Light intensity was approximately 250 μmol m-2 s-1 and temperatures were 26° C. during the day and 18° C. at night.

b) siRNA Sequences (According to Hendrix B, et al. (2021) PLOS ONE 16(3): E0245422) the siRNA Sequences Employed are:

GFP16C_sense:
(SEQ ID NO: 1)
5′ GGCAUCAAAGCCAACUUCAAAA 3′
GFP16C_antisense:
(SEQ ID NO: 2)
5′ UUGAAGUUGGCUUUGAUGCCGU 3′

All siRNAs (20-22 nt) were synthesized by IDT (Integrated DNA Technologies) with 2 nt 3′ overhangs.

### c) Experimental Design

Abrasion-based delivery of siRNAs to plant leaves N. benthamiana was conducted essentially as described (Hendrix et al., Planta 254: 60 (2021). An aliquot of 20-25 μl of a treatment solution was pipetted onto the adaxial surface of 2-3 partially expanded leaves of 14-21-day-old N. benthamiana plants, covering the totality of the leaf surfaces. After the solution had dried, the adaxial surface of the leaf was lightly abraded using 600 grit sandpaper affixed to a wooden dowel (7 cm long, 1.2 cm diameter). The abrasion was achieved with a gently rolling motion on the treated leaf surface. The goal of this procedure was to lightly abrade the adaxial surface of the leaf without crushing or tearing the leaf. The leaves may wilt after abrasion but will recover if inner leaf tissues are not damaged to the point of cell lysis and death.

The treatment solutions were as follows: 1) NLP empty (no RNA); 2) rNLP containing siRNA; and 3) unencapsulated siRNA solution comprising 1 μg/μl siRNA in 0.01% Silwet L77.

GFP fluorescence of plants was visualized after 48 hours of treatment using a UV light and images were captured using a NIKON camera.

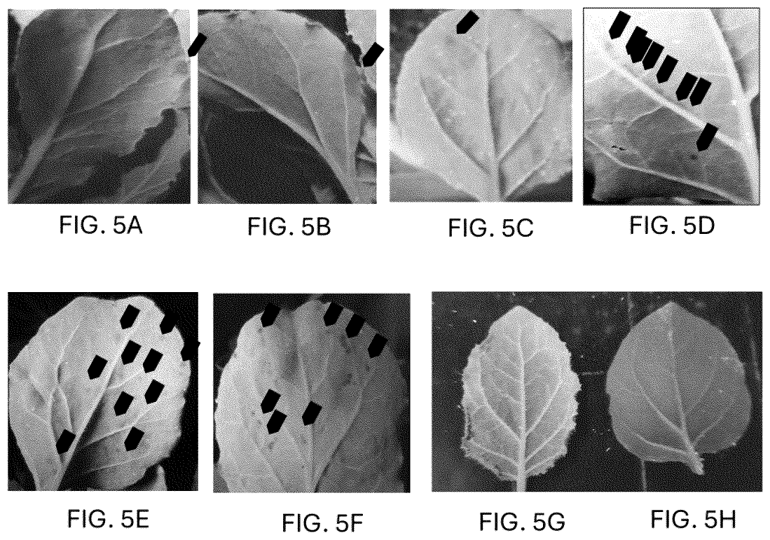

Results: Results are shown in FIG. 5A-5H. With select rNLP formulation containing siRNA-GFP the expected phenotype corresponding to spots of reduced GFP fluorescence was observed.

### Example 24. Uptake of rNLP Containing gRNA by Mechanical Uptake in Cas9-Ox Nicotiana Plants

These experiments study the uptake and the efficacy of the CRISPR machinery using NLPs.

### Experimental Procedure

Transgenic Cas-overexpressing N. benthamiana plants are employed. For the design of sgRNAs, N. benthamiana genes chosen as targets for CRISPR-Cas9-mediated gene are NbFT and NbPDS3. These gRNA are encapsulated in rNLPs and entered in the plant using inoculation in the leaves with a needleless syringe. As a positive control, transgenic Cas9-expressing N. benthamiana plants transformed via Agrobacterium with NbFt and NbPDS3 gRNAs cloned in TRV vectors.

Results: Nicotiana Cas9-ox plants treated with rNLP containing sgRNA for NbPSD3 and NbFT will show a photobleching phenotype in the leaves. Positive controls: Agro-inoculation of a Cas9-ox plants with sgRNA of NbFt and NbPDS3 gRNAs cloned in TRV vectors will result in a leaf mosaic with green and photobleached sectors. The phenotype will increase in intensity as the plants matured, and will be absent in wildtype (negative control). Negative controls are Nicotiana Cas9-ox plants treated with empty NLPS and plants inoculated with the empty TRV vectors, that show now bleaching effects

### Example 25: Uptake of rNLP Containing gRNA in Cas9 Arabidopsis Plants

This example describes the uptake and function of rNLP-gRNA in planta after floral dip in Arabidopsis. These experiments study the uptake and the efficacy of the CRISPR machinery using rNLPs.

### Experimental Procedure

For the encapsulation of different gRNAs for targeting transparent testa (TT) genes TT3, TT4 and TT5, healthy Arabidopsis plants are grown until they are flowering under long days conditions. The first bolts are cut to encourage proliferation of many secondary bolts. An aliquot of 50 ul of treatment solution is applied and repeated 2 days later. The effects on the color of the seeds is analyzed in the stereomicroscope Olympus SZX16. Treatment solutions are rNLP-gRNA and appropriate controls, e.g. 5% sucrose 0.05% silwett L-77 rNLP-RNA solution.

Results: A percentage of seeds coming from flowers inoculated with rNLP-gRNA(TT) present a transparent seed coat showing yellow color instead of brown. Positive controls performed with agrobacterium containing the gRNA of 7T also present a percentage of yellow seeds. Negative controls, e.g. NLP-empty and water present brown seeds.

### Example 26: Uptake of rNLP Containing gRNA in Cas9 Tomato Plants

This example describes the uptake and function of rNLP-gRNA in tomato. These experiments study the uptake and the efficacy of the CRISPR machinery using rNLPs.

### Experimental Procedure

Tomato plants (Solanum lycopersicum L.) cv. Micro-Tom will be used to generate CRISPR/Cas9 edited plants with rNLPs using leaf disk method. Briefly, cut cotyledon pieces (7-10 days after seeding) will be incubated combinations of two rNLP populations (example 6, table 11) One rNLP will contain a sgRNA for SlIAA9 gene and the other the Cas9 mRNA. RNAs will be encapsulated in select rNLPs and entered in the plant using inoculation in the leaves with a needleless syringe

Results: CRISPR_Cas9 edited plants will show a parthenocarpic phenotype (leaf shape, seedless). Positive controls: Micro TOM plants will be transformed with A. tumefaciens strain GV2260 harboring the CRISPR/Cas9 plasmid containing sgRNA for SlIAA9 gene. Edited plants will show parthenocarpic phenotype. Negative controls: Micro-TOM plants treated with empty NLPS and inoculated with the empty CRISPR-Cas9 vectors do not show the parthenocarpic phenotype.

### Example 27: Uptake of rNLP Containing gRNA in Cas9 Corn Plants

This example describes the uptake and function of rNLP-gRNA in corn. These experiments study the uptake and the efficacy of the CRISPR machinery using rNLPs.

### Experimental Procedure

For this experiment, transgenic Cas9-expressinB104 maize plants are employed. For the design of sgRNAs, maize targets for CRISPR-Cas9-mediated gene are zmVYL or similar, leading to albino phenotype. gRNAs will be encapsulated in select rNLPs and entered in the plant using inoculation in the leaves with a needleless syringe. Morphogenic genes as Wuschel or BBm2 are also introduced for efficient maize transformation. As a positive control, B104 plants are transformed via Agrobacterium with gRNAs cloned in maize binary vectors.

For this experiment, transgenic Cas9-expressing B104 maize plants will be employed. A combination of rNLP population will be needed. 1 population of rNLP s will contain sgRNA for zmVYL1 gene. Plasmids for overexpression of morphogenic genes as Wuschel or BBm2 will be also encapsulated in a different NLPs population, introduced for efficient maize transformation and removed using Cre/LoxP-mediated excision system after transformation

Results: CRISPR-Cas9 edited plants with zmWYL1 gene will present with albino phenotype. As a positive control, B 104 plants are transformed via Agrobacterium with gRNA zmWYL1 cloned in maize binary vectors. Negative controls: B 104 plants treated with empty NLPS and inoculated with the empty CRISPR-Cas9 vectors.

### Example 28: Composition and Physicochemical Characteristics of NLPs Produced

80 new NLP formulations were produced as described in Example 4 from 15 different commercially available lipids (7 ionizable/cationic lipids, 5 phospholipids (some from plants sources), Cholesterol and 2 PEG lipids) according to the lipid ratios described in Table 17 below.

TABLE 17
NLP constituents

| Lipid Ratio | 14-40% | 40-60% | | 0-1.5% | 0-25% |
|---|---|---|---|---|---|
| Lipid Type | Sterol | Ionizable Lipid | Cationic Lipid | PEG-Lipid | Phospholipid |
| | Cholesterol | SM-102 | DDAB | PEG 5000 | Sunflower |
| | | | | | lecithin |
| | | DODAP | DOTAP | DMG-PEG | DOPE |
| | | | | 2000 | |
| | | | DODMA | | MGDG |
| | | | DLin-MC3- | | DSPC |
| | | | DMA | | |
| | | | DLin-KC2- | | Hydro Soy PC |
| | | | DMA | | |

NLP formation was verified by electron and cryo-electron microscopy on a JEOL 1010 transmission electron microscope, following the protocol from Wu et al., Analyst. 140(2):386-406, 2015. The NLP particle size, NLP particle size distribution, and zeta potential were measured immediately after production using a Malvern Panalytical Zetasizer, following the manufacturer's instructions. The encapsulation efficiency (EE %) was calculated by the (initial concentration of polynucleotide added/actual concentration of polynucleotide in the NLP)*100%, immediately after production.

TABLE 18
Physicochemical characteristics of NLPs produced

| | | | | Zeta | | |
|---|---|---|---|---|---|---|
| | | | | potential | | |
| ID | Form. | Size (nm) | PDI | (mV) | EE % | N:P Ratio |
| rNLP_S22 | 167 | 158 | 0.237 | 11.3 | 95.12 | |
| rNLP_S23 | 189 | 136.7 | 0.182 | 10.6 | 67.07 | 6 |
| rNLP_S24 | 190 | 120.1 | 0.278 | 15.7 | 67.49 | 6 |
| rNLP_S25 | 191 | 246.4 | 0.638 | 12.73 | 67.94 | 6 |
| rNLP_S26 | 192 | 185.2 | 0.205 | 5.45 | 52.39 | 5 |
| rNLP_S27 | 193 | 467.3 | 0.116 | 19.96 | 31.82 | 7.5 |
| rNLP_S28 | 199 | 133 | 0.269 | 11.4 | 91.11 | 3 |
| rNLP_S29 | 200 | — | — | — | — | 7.5 |
| rNLP_S30 | 201 | 230.7 | 0.405 | — | 93.53 | 7.5 |
| rNLP_S31 | 202 | — | — | — | — | 7.5 |
| rNLP_S32 | 203 | — | — | — | — | 3 |
| rNLP_S33 | 209 | 180.3 | 0.188 | 5.37 | 93.91 | 7.5 |
| rNLP_S34 | 210 | 160.4 | 0.333 | 37.6 | 97.13 | 7.5 |
| rNLP_S35 | 211 | 249.8 | 0.239 | 21.73 | 91.1 | 3 |
| rNLP_S36 | 212 | 310 | 0.078 | 29.7 | 79.2 | 7.5 |
| rNLP_S37 | 216 | 111.9 | 0.239 | 3.39 | 52.78 | 7.5 |
| rNLP_S38 | 217 | 191.9 | 0.389 | 5.86 | 50.18 | 3 |
| rNLP_S39 | 218 | 225 | 0.253 | 18.56 | 21.47 | 3 |
| rNLP_S40 | 219 | 173.9 | 0.107 | 20.03 | 42.95 | 7.5 |
| rNLP_S41 | 220 | 85.87 | 0.163 | 1.095 | 53.41 | 7.5 |
| rNLP_S42 | 221 | 214.5 | 0.51 | — | 54.25 | 7.5 |
| rNLP_S43 | 222 | 381.2 | 0.116 | — | 54.17 | 3 |
| rNLP_S44 | 223 | 212.5 | 0.452 | — | 52.9 | 3 |
| rNLP_S45 | 224 | 194.4 | 0.103 | — | 53.84 | 3 |
| rNLP_S46 | 225 | 764.2 | 0.538 | — | 54.86 | 3 |
| rNLP_S47 | 228 | 123.8 | 0.375 | 3.05 | 86.17 | 7.5 |
| rNLP_S48 | 229 | 141.4 | 0.109 | 3.53 | 87.76 | 3 |
| rNLP_S49 | 230 | 161 | 0.357 | 29.4 | 76.05 | 3 |
| rNLP_S50 | 231 | 132.4 | 0.323 | 2.36 | 80.07 | 3 |
| rNLP_S51 | 232 | — | — | — | 72.51 | 3 |
| rNLP_S52 | 233 | 118.4 | 0.358 | 3.85 | 80.87 | 7.5 |
| rNLP_S53 | 237 | 187.9 | 0.21 | 10.5 | 91.66 | 7.5 |
| rNLP_S54 | 238 | 262.3 | 0.16 | 24.1 | 82.86 | 3 |
| rNLP_S55 | 239 | 167.4 | 0.091 | 6.83 | 83.18 | 7.5 |
| rNLP_S56 | 240 | 1608 | 1 | 32.5 | 85.78 | 3 |
| rNLP_S57 | 241 | 799.5 | 0.288 | 30.7 | 80.96 | 5 |
| rNLP_S58 | 242 | 115.2 | 0.272 | 12.7 | 78.42 | 7.5 |
| rNLP_S59 | 243 | 131.3 | 0.211 | — | 80.95 | 3 |
| rNLP_S60 | 244 | 325.2 | 0.044 | — | 95.94 | 3 |
| rNLP_S61 | 245 | 1733.1 | 0.255 | 1.08 | 91.12 | 7.5 |
| rNLP_S62 | 246 | 18520 | 0.714 | 16.97 | 84.39 | 3 |
| rNLP_S63 | 247 | 175.4 | 0.368 | 0.352 | 96.01 | 7.5 |
| rNLP_S64 | 248 | 2134 | 0.888 | 24.73 | 88.21 | 7.5 |
| rNLP_S65 | 249 | 112.3 | 0.198 | 7.02 | 93.01 | 7.5 |
| rNLP_S66 | 250 | 113.3 | 0.168 | 8.72 | 91.38 | 7.5 |
| rNLP_S67 | 251 | 190.2 | 0.118 | 12.4 | 90.23 | 3 |
| rNLP_S68 | 252 | 252.1 | 0.152 | 7.91 | 77.96 | 7.5 |
| rNLP_S69 | 253 | 112.8 | 0.378 | 0.973 | 81.71 | 7.5 |
| rNLP_S70 | 254 | 304.8 | 0.28 | 26.8 | 81.15 | 7.5 |
| rNLP_S71 | 259 | 105.7 | 0.292 | 1.99 | 91.87 | 3 |
| rNLP_S72 | 270 | 170.4 | 0.156 | 28.8 | 92.05 | 3 |
| rNLP_S73 | 272 | 188.7 | 0.355 | 8.7 | 93.46 | 3 |
| rNLP_S74 | 271 | 1187 | 0.797 | 24.06 | 91.67 | 3 |
| rNLP_S75 | 273 | 256 | 0.294 | — | 66.65 | 3 |
| rNLP_S76 | 274 | 676.2 | 0.254 | — | 65.24 | 7.5 |
| rNLP_S77 | 275 | — | — | — | — | 3 |
| rNLP_S78 | 276 | — | — | — | — | 7.5 |
| rNLP_S79 | 277 | — | — | — | — | 7.5 |
| rNLP_S80 | 330 | — | — | — | — | 7.5 |
| rNLP_S81 | 331 | — | — | — | — | 7.5 |
| rNLP_S82 | 332 | — | — | — | — | 3 |
| dNLP_S54 | 365 | — | — | — | — | 7.5 |
| dNLP_S55 | 366 | — | — | — | — | 7.5 |
| dNLP_S56 | 367 | — | — | — | — | 3 |
| dNLP_S57 | 368 | — | — | — | — | 7.5 |
| dNLP_S58 | 369 | — | — | — | — | 3 |
| dNLP_S59 | 370 | — | — | — | — | 3 |
| dNLP_S60 | 371 | — | — | — | — | 7.5 |
| dNLP_S61 | 372 | — | — | — | — | 3 |
| dNLP_S62 | 373 | — | — | — | — | 7.5 |
| dNLP_S63 | 374 | — | — | — | — | 7.5 |
| dNLP_S64 | 375 | — | — | — | — | 7.5 |
| dNLP_S65 | 376 | — | — | — | — | 7.5 |
| dNLP_S66 | 377 | 4873 | 1 | −8.028 | 33.33 | 3 |
| dNLP_S67 | 378 | 136.7 | 0.252 | 0.223 | 92.7 | 7.5 |
| dNLP_S68 | 379 | — | — | — | — | 5 |
| dNLP_S69 | 380 | — | — | — | — | 7.5 |
| dNLP_S70 | 381 | — | — | — | — | 3 |
| dNLP_S71 | 382 | — | — | — | — | 7.5 |
| dNLP_S72 | 383 | — | — | — | — | 3 |

The molar percentages (mol %) of the various constituents of the lipid components of the NLPs are indicated in Table 19. The mol % are calculated first by calculating the moles of every component in the formulation by taking into account the amount of the component added into the formulation (uL) and the concentration of the stock solution (e.g., 5 ug/uL) and the molecular weight of the corresponding component. Then the mole % is obtained taking into account the moles of every component in the formulation with the total number of moles of all liposome constituents combined in the specific formulation.

TABLE 19
Composition (molar percentages) of NLPs produced

| | | | Ionizable | Cationic | | |
|---|---|---|---|---|---|---|
| ID | Form. | Chol | Lipid | Lipid | PEG Lipid | Phospholipid |
| rNLP_S22 | 167 | 38.5 | | 50 MC3 | 1.5 PEG2K PE 18:0 | 10 DSPC |
| rNLP_S23 | 189 | 38.5 | 50 SM-102 | | 1.5 PEG2K PE 18:0 | 10 DSPC |
| rNLP_S24 | 190 | 38.5 | | 50 KC2 | 1.5 PEG2K PE 18:0 | 10 DSPC |
| rNLP_S25 | 191 | 38.5 | | 50 DOTAP | 1.5 PEG2K PE 18:0 | 10 DSPC |
| rNLP_S26 | 192 | 39.25 | 60 SM-102 | | 0.7 PEG2K PE 18:0 | |
| rNLP_S27 | 193 | 35 | | 40 KC2 | | 25 DSPC |
| rNLP_S28 | 199 | 20 | 60 DODAP | | 1.5 PEG2K PE 18:0 | 18.5 DOPE |
| rNLP_S29 | 200 | 40 | 41.85 SM-102 | | 1.5 PEG5K PE 14:0 | 16.65 MGDG |
| rNLP_S30 | 201 | 20 | | 54.19 DODMA | 0.81 PEG5K PE 14:0 | 25 MGDG |
| rNLP_S31 | 202 | 38.5 | | 60 MC3 | 1.5 PEG5K PE 14:0 | |
| rNLP_S32 | 203 | 40 | | 40 KC2 | 0.75 PEG2K PE 18:0 | 19.25 Hydro Soy PC |
| rNLP_S33 | 209 | 40 | | 59.4 MC3 | 0.6 PEG2K PE 18:0 | |
| rNLP_S34 | 210 | 20 | | 60 DOTAP | | 20 Hydro Soy PC |
| rNLP_S35 | 211 | 40 | | 40 DOTAP | | 20 DSPC |
| rNLP_S36 | 212 | 40 | 40 SM-102 | | | 20 DSPC |
| rNLP_S37 | 216 | 40 | | 58.5 DOTAP | 1.5 PEG5K PE 14:0 | |
| rNLP_S38 | 217 | 38.5 | | 60 DOTAP | 1.5 PEG2K PE 18:0 | |
| rNLP_S39 | 218 | 35 | | 40 DODMA | | 25 DSPC |
| rNLP_S40 | 219 | 40 | 40 DODAP | | | 20 Hydro Soy PC |
| rNLP_S41 | 220 | 40 | 58.5 DODAP | | 1.5 PEG5K PE 14:0 | |
| rNLP_S42 | 221 | 40 | | 60 DODMA | | |
| rNLP_S43 | 222 | 35 | | 40 DDAB | | 25 SF lecithin |
| rNLP_S44 | 223 | 20 | | 53.5 DODMA | 1.5 PEG5K PE 14:0 | 25 SF lecithin |
| rNLP_S45 | 224 | 35 | | 40 MC3 | | 25 Hydro Soy PC |
| rNLP_S46 | 225 | 40 | | 58.5 MC3 | 1.5 PEG2K PE 18:0 | |
| rNLP_S47 | 228 | 20 | | 60 MC3 | 1.5 PEG5K PE 14:0 | 18.5 MGDG |
| rNLP_S48 | 229 | 40 | | 58.5 DDAB | 1.5 PEG2K PE 18:0 | |
| rNLP_S49 | 230 | 40 | 40 DODAP | | | 20 SF lecithin |
| rNLP_S50 | 231 | 38.5 | 60 DODAP | | 1.5 PEG5K PE 14:0 | |
| rNLP_S51 | 232 | 40 | | 60 MC3 | | |
| rNLP_S52 | 233 | 20 | 60 DODAP | | 1.5 PEG5K PE 14:0 | 18.5 DSPC |
| rNLP_S53 | 237 | 39.4 | 60 SM-102 | | 0.6 PEG2K PE 18:0 | |
| rNLP_S54 | 238 | 40 | | 56 DDAB | | 4 DOPE |
| rNLP_S55 | 239 | 40 | | 40 DDAB | 1.5 PEG2K PE 18:0 | 18.5 DSPC |
| rNLP_S56 | 240 | 40 | 60 SM-102 | | | |
| rNLP_S57 | 241 | 20 | | 60 DDAB | | 20 Hydro Soy PC |
| rNLP_S58 | 242 | 20 | | 54.232 MC3 | 0.768 PEG2K PE 18:0 | 25 Hydro Soy PC |
| rNLP_S59 | 243 | 20 | | 60 KC2 | 1.5 PEG5K PE 14:0 | 18.5 DSPC |
| rNLP_S60 | 244 | 40 | | 40 KC2 | | 20 SF lecithin |
| rNLP_S61 | 245 | 33.5 | 40 DODPA | | 1.5 PEG5K PE 14:0 | 25 SF lecithin |
| rNLP_S62 | 246 | 40 | | 40 DODMA | | 20 MGDG |
| rNLP_S63 | 247 | 34.8 | | 40 KC2 | 1.5 PEG5K PE 14:0 | 23.7 DOPE |
| rNLP_S64 | 248 | 40 | | 60 KC2 | | |
| rNLP_S65 | 249 | 40 | 40 DODAP | | 1.5 PEG2K PE 18:0 | 18.5 MGDG |
| rNLP_S66 | 250 | 20 | 58.4 SM-102 | | 1.5 PEG2K PE 18:0 | 20.056 DOPE |
| rNLP_S67 | 251 | 34.7 | | 40 MC3 | 0.3 PEG2K PE 18:0 | 25 MGDG |
| rNLP_S68 | 252 | 28 | | 60 MC3 | | 12 DSPC |
| rNLP_S69 | 253 | 40 | | 49.25 MC3 | 1.5 PEG5K PE 14:0 | 9.25 Hydro Soy PC |
| rNLP_S70 | 254 | 40 | | 40 MC3 | | 20 SF lecithin |
| rNLP_S71 | 259 | 31.1 | | 60 MC3 | 1.5 PEG5K PE 14:0 | 7.4 DOPE |
| rNLP_S72 | 270 | 20 | | 55 MC3 | | 25 DSPC |
| rNLP_S73 | 272 | 20 | | 60 MC3 | 0.9 PEG2K PE 18:0 | 19.1 SF lecithin |
| rNLP_S74 | 271 | 40 | | 60 MC3 | | |
| rNLP_S75 | 273 | 33.5 | | 40 MC3 | 1.5 PEG2K PE 18:0 | 25 DOPE |
| rNLP_S76 | 274 | 30.5 | | 44.5 DOTAP | | 25 MGDG |
| rNLP_S77 | 275 | 20 | | 55 DDAB | | 25 DSPC |
| rNLP_S78 | 276 | 38.5 | | 60 DDAB | 1.5 PEG5K PE 14:0 | |
| rNLP_S79 | 277 | 40 | | 60 DDAB | | |
| rNLP_S80 | 330 | 20 | | 53.5 DDAB | 1.5 PEG2K PE 18:0 | 25 SF lecithin |
| rNLP_S81 | 331 | 20 | | 60 MC3 | | 20 DOPE |
| rNLP_S82 | 332 | 20 | | 53.5 DDAB | 1.5 PEG5K PE 14:0 | 25 MGDG |
| dNLP_S54 | 365 | 40 | | 40 DDAB | | 20 Hydro Soy PC |
| dNLP_S55 | 366 | 40 | | 40 DDAB | | 20 MGDG |
| dNLP_S56 | 367 | 33.5 | | 40 DDAB | 1.5 PEG2K PE 18:0 | 25 DOPE |
| dNLP_S57 | 368 | 33.5 | | 40 MC3 | 1.5 PEG5K PE 14:0 | 25 DSPC |
| dNLP_S58 | 369 | 33.5 | | 40 DOTAP | 1.5 PEG2K PE 18:0 | 25 Hydro Soy PC |
| dNLP_S59 | 370 | 40 | | 51.1 DOTAP | 1.5 PEG5K PE 14:0 | 7.4 SF lecithin |
| dNLP_S60 | 371 | 20 | | 60 DOTAP | | 20 DSPC |
| dNLP_S61 | 372 | 40 | | 60 DOTAP | | |
| dNLP_S62 | 373 | 40 | | 59.1 DOTAP | 0.9 PEG2K PE 18:0 | |
| dNLP_S63 | 374 | 35 | | 40 DOTAP | | 25 SF lecithin |
| dNLP_S64 | 375 | 20 | | 60 DOTAP | 1.5 PEG5K PE 14:0 | 18.5 SF lecithin |
| dNLP_S65 | 376 | 40 | | 40 DOTAP | 1.5 PEG5K PE 14:0 | 18.5 DOPE |
| dNLP_S66 | 377 | 20 | | 60 DOTAP | | 20 MGDG |
| dNLP_S67 | 378 | 20 | | 53.5 DDAB | 1.5 PEG5K PE 14:0 | 25 DOPE |
| dNLP_S68 | 379 | 40 | | 60 KC2 | | |
| dNLP_S69 | 380 | 33.5 | | 40 DODMA | 1.5 PEG2K PE 18:0 | 25 Hydro Soy PC |
| dNLP_S70 | 381 | 25.55 | | 60 DODMA | 1.5 PEG2K PE 18:0 | 12.95 MGDG |
| dNLP_S71 | 382 | 40 | | 58.5 DODMA | 1.5 PEG2K PE 18:0 | |
| dNLP_S72 | 383 | 40 | | 43.7 DODMA | 1.5 PEG5K PE 14:0 | 14.8 DOPE |

The initial loading concentration of polynucleotide cargo of several NLP formulations as described herein as well as the Encapsulation Efficiency (EE %) (Amount of Cargo Incorporated/Initial Amount of Cargo Loaded) is provided in Table 20. Incorporation of polynucleotide cargo in the rNLPs was confirmed by quantifying RNA concentration by Quant-it™ RiboGreen RNA Assay Kit (Invitrogen) according to manufacturer's protocol. The Quant-iT™ RiboGreen™ RNA method is a highly sensitive and specific fluorescent assay for quantifying RNA in solution. It utilizes the RiboGreen™ dye, which selectively binds to RNA, producing a strong fluorescence signal proportional to the RNA concentration. This method allows for accurate RNA measurement even in the presence of contaminants like DNA and proteins. The concentration of cargo is expressed per ml of rNLP suspension.

TABLE 20
Cargo incorporation

| | | | | Nluc | | | |
|---|---|---|---|---|---|---|---|
| | | | GFP | RNA | | | |
| | | | RNA | GFP | Nluc RNA | SiRNA-GFP | DNA |
| ID | Form. | EE % | (ug/ml) | (ug/ul) | Cyan (ug/ul) | (ug/mL) | (ug/ul) |
| rNLP_S22 | 167 | 95.12 | | 68 | 68 | | |
| rNLP_S23 | 189 | 67.07 | | 68 | 68 | | |
| rNLP_S24 | 190 | 67.49 | | 68 | 68 | | |
| rNLP_S25 | 191 | 67.94 | 68 | 68 | 68 | | |
| rNLP_S26 | 192 | 52.39 | 40 | 40 | 40 | | |
| rNLP_S27 | 193 | 31.82 | 40 | 40 | 40 | | |
| rNLP_S28 | 199 | 91.11 | 40 | 40 | 40 | | |
| rNLP_S29 | 200 | Not Calc. | 40 | 40 | 40 | | |
| rNLP_S30 | 201 | 93.53 | 40 | 40 | 40 | | |
| rNLP_S31 | 202 | Not Calc. | 40 | 40 | 40 | | |
| rNLP_S32 | 203 | Not Calc. | 40 | 40 | 40 | | |
| rNLP_S33 | 209 | 93.91 | 40 | 40 | 40 | 50 | |
| rNLP_S34 | 210 | 97.13 | 40 | 40 | 40 | | |
| rNLP_S35 | 211 | 91.1 | 40 | 40 | 40 | 50 | |
| rNLP_S36 | 212 | 79.2 | 40 | 40 | 40 | | |
| rNLP_S37 | 216 | 52.78 | 40 | 40 | 40 | | |
| rNLP_S38 | 217 | 50.18 | 40 | 40 | 40 | | |
| rNLP_S39 | 218 | 21.47 | 40 | 40 | 40 | | |
| rNLP_S40 | 219 | 42.95 | 40 | 40 | 40 | | |
| rNLP_S41 | 220 | 53.41 | 40 | 40 | 40 | | |
| rNLP_S42 | 221 | 54.25 | 40 | 40 | 40 | | |
| rNLP_S43 | 222 | 54.17 | 40 | 40 | 40 | | |
| rNLP_S44 | 223 | 52.9 | 40 | 40 | 40 | | |
| rNLP_S45 | 224 | 53.84 | 40 | 40 | 40 | | |
| rNLP_S46 | 225 | 54.86 | 40 | 40 | 40 | | |
| rNLP_S47 | 228 | 86.17 | 40 | 40 | 40 | | |
| rNLP_S48 | 229 | 87.76 | 40 | 40 | 40 | | |
| rNLP_S49 | 230 | 76.05 | 40 | 40 | 40 | | |
| rNLP_S50 | 231 | 80.07 | 40 | 40 | 40 | | |
| rNLP_S51 | 232 | 72.51 | 40 | 40 | 40 | | |
| rNLP_S52 | 233 | 80.87 | 40 | 40 | 40 | | |
| rNLP_S53 | 237 | 91.66 | 40 | 40 | 40 | | |
| rNLP_S54 | 238 | 82.86 | 40 | 40 | 40 | | |
| rNLP_S55 | 239 | 83.18 | 40 | 40 | 40 | | |
| rNLP_S56 | 240 | 85.78 | 40 | 40 | 40 | | |
| rNLP_S57 | 241 | 80.96 | 40 | 40 | 40 | | |
| rNLP_S58 | 242 | 78.42 | 40 | 40 | 40 | | |
| rNLP_S59 | 243 | 80.95 | 40 | 40 | 40 | | |
| rNLP_S60 | 244 | 95.94 | 40 | 40 | 40 | | |
| rNLP_S61 | 245 | 91.12 | 40 | 40 | 40 | | |
| rNLP_S62 | 246 | 84.39 | 40 | 40 | 40 | | |
| rNLP_S63 | 247 | 96.01 | 40 | 40 | 40 | | |
| rNLP_S64 | 248 | 88.21 | 40 | 40 | 40 | | |
| rNLP_S65 | 249 | 93.01 | 40 | 40 | 40 | | |
| rNLP_S66 | 250 | 91.38 | 40 | 40 | 40 | | |
| rNLP_S67 | 251 | 90.23 | 40 | 40 | 40 | | |
| rNLP_S68 | 252 | 77.96 | 40 | 40 | 40 | | |
| rNLP_S69 | 253 | 81.71 | 40 | 40 | 40 | | |
| rNLP_S70 | 254 | 81.15 | 40 | 40 | 40 | | |
| rNLP_S71 | 259 | 91.87 | 40 | 40 | 40 | | |
| rNLP_S72 | 270 | 92.05 | 40 | 40 | 40 | | |
| rNLP_S73 | 272 | 93.46 | 40 | 40 | 40 | | |
| rNLP_S74 | 271 | 91.67 | 40 | 40 | 40 | | |
| rNLP_S75 | 273 | 66.65 | 40 | 40 | 40 | | |
| rNLP_S76 | 274 | 65.24 | 40 | 40 | 40 | | |
| rNLP_S77 | 275 | Not Calc. | 40 | 40 | 40 | | |
| rNLP_S78 | 276 | Not Calc. | 40 | 40 | 40 | | |
| rNLP_S79 | 277 | Not Calc. | 40 | 40 | 40 | | |
| rNLP_S80 | 330 | Not Calc. | 40 | 40 | 40 | | |
| rNLP_S81 | 331 | Not Calc. | 40 | 40 | 40 | | |
| rNLP_S82 | 332 | Not Calc. | 40 | 40 | 40 | | |
| dNLP_S54 | 365 | Not Calc. | | | | | 20 |
| dNLP_S55 | 366 | Not Calc. | | | | | 20 |
| dNLP_S56 | 367 | Not Calc. | | | | | 20 |
| dNLP_S57 | 368 | Not Calc. | | | | | 20 |
| dNLP_S58 | 369 | Not Calc. | | | | | 20 |
| dNLP_S59 | 370 | Not Calc. | | | | | 20 |
| dNLP_S60 | 371 | Not Calc. | | | | | 20 |
| dNLP_S61 | 372 | Not Calc. | | | | | 20 |
| dNLP_S62 | 373 | Not Calc. | | | | | 20 |
| dNLP_S63 | 374 | Not Calc. | | | | | 20 |
| dNLP_S64 | 375 | Not Calc. | | | | | 20 |
| dNLP_S65 | 376 | Not Calc. | | | | | 20 |
| dNLP_S66 | 377 | 33.33 | 40 | | | | 20 |
| dNLP_S67 | 378 | 92.7 | 40 | | | | 20 |
| dNLP_S68 | 379 | Not Calc. | | | | | 20 |
| dNLP_S69 | 380 | Not Calc. | | | | | 20 |
| dNLP_S70 | 381 | Not Calc. | | | | | 20 |
| dNLP_S71 | 382 | Not Calc. | | | | | 20 |
| dNLP_S72 | 383 | Not Calc. | | | | | 20 |

### Example 29. Local Uptake of NLP Formulation in Arabidopsis Seedlings and Corn Leaves

Uptake and subcellular localization of RNA cargo encapsulated in selected NLP formulations produced as described in Example 28 above was examined in Arabidopsis thaliana and Corn leaves.

Experimental Procedure for Assaying the Uptake of rNLP Formulations into Arabidopsis thaliana Leaves:

### a) Seed Sterilization and Plating

Arabidopsis seeds were surface sterilized with bleach solution (30% commercial bleach) for 5 minutes, washed 5 times with sterile water, and placed at 4° C. for 48 hours prior to plating. Seeds were plated in squared petri dishes onto 0.5× strength Murashige and Skoog salt mixture (0.5×MS) including vitamins and without sucrose (Duchefa, Cat. n #M0222) and 0.8% plant agar (Duchefa Cat. n #P1001) and grown for twelve days in long day at 22° C.

b) rNLP Treatments

Twelve-day-old seedlings were transferred to 24-well plates containing 400 μl of either (a) 0.5×MS media, (b) an empty NLP formulation, or (c) an rNLP formulation comprising an mRNA cargo of mTurquoise-NLS sequence. Incubations were carried out for 6 to 24 hours. After 6 to 24h of incubation, true leaves of the Arabidopsis rosette were cut and placed in microscopy slides to be imaged in their abaxial side. Leaves were visualized by confocal microscopy using a Zeiss Elyra 980 microscope using GFP settings (Excitation 445 nm, emission 496 nm). Epidermal pavement cells were observed. As shown in Table 21, GFP expression was detected in Arabidopsis leaves for 37 of the 53 rNLPs tested, indicating uptake and translation of the GFP RNA cargo.

Experimental Procedure for Assaying the Uptake of rNLP Formulations into Corn Leaves:

### a) Seed Sterilization

Corn seeds were placed in a 500 ml beaker containing 1 magnet for stirring and 250 ml of ethanol 70%. The seeds were incubated by stirring for 2 minutes. The ethanol was then removed, and 250 ml of 50% commercial bleach was added with a drop of triton X-20 and stirred for 30 minutes. The bleach solution was removed followed by five washed with autoclaved miliQ water of 10 min each while stirring.

### b) Seed Germination

Filter paper sheets were autoclaved, followed by the addition of 5 ml of 4% preservative for plant culture media (PPM™) comprising 5-chloro-2-methyl-3(2H)-isothiazolone and 2-methyl-3(2H)-isothiazolone (Plant Cell Technologies, Inc), diluted in sterile MiliQ water. Ten seeds were placed per roll and secured with an elastic rubberband, 6 rolls were placed in a 1-liter beaker containing 200 ml of 4% PMM diluted in sterile water. Beakers were then wrapped in foil and placed in a 25° C. growth chamber for three weeks.

c) rNLP Treatment of Corn Seedlings

Treatments of corn seedlings were performed in 24-well-plates. 500 ul aliquots of (a) rNLP formulations comprising RNA cargo (GFP RNA, NLS-mTurquoise or others); (b) empty (no Cargo) NLP formulations (Neg. Ctrl.); or (c) m0.5 MS buffer diluted twice in transfection buffer (10% sucrose, 20 mM MgCl2 and 0.01% Silwet were added in each well such that at least 3 sections of 1 cm² leaves were placed in contact with the treatment solution. Plates were incubated for 16-24 hours at 25° C.

### d) Uptake Visualization in Corn Leaf Tissue by Using Confocal Microscopy Imaging

Confocal microscopy visualization of the epidermal layer of the corn leaves for GFP was performed using a Zeiss 980 Elyra 7; Olympus FV1000) with excitation/emission wavelengths settings of Excitation 445 nm, emission 496 nm. As shown in Table 21, GFP was detected in corn leaves incubated in FORM167, FORM199, FORM201, FORM209, FORM211, FORM245, FORM249, FORM251 and FORM253.

TABLE 21
Local Uptake of rNLP formulation in Arabidopsis and Corn Leaves

| | | GFP detected in | GFP detected |
|---|---|---|---|
| ID | Form. | Arabidopsis | in Corn |
| rNLP_S22 | 167 | Yes | Yes |
| rNLP_S23 | 189 | No | Not performed |
| rNLP_S24 | 190 | No | Not performed |
| rNLP_S25 | 191 | No | Not performed |
| rNLP_S26 | 192 | No | Not performed |
| rNLP_S27 | 193 | No | Not performed |
| rNLP_S28 | 199 | Yes | Yes |
| rNLP_S29 | 200 | Yes | Not performed |
| rNLP_S30 | 201 | Yes | Yes |
| rNLP_S31 | 202 | No | Not performed |
| rNLP_S32 | 203 | Yes | No |
| rNLP_S33 | 209 | Yes | Yes |
| rNLP_S34 | 210 | Yes | Not performed |
| rNLP_S35 | 211 | Yes | Yes |
| rNLP_S36 | 212 | Yes | Not performed |
| rNLP_S37 | 216 | No | Not performed |
| rNLP_S38 | 217 | No | Not performed |
| rNLP_S39 | 218 | No | Not performed |
| rNLP_S40 | 219 | Yes | Not performed |
| rNLP_S41 | 220 | Yes | Not performed |
| rNLP_S42 | 221 | Yes | Not performed |
| rNLP_S43 | 222 | Yes | Not performed |
| rNLP_S44 | 223 | Yes | Not performed |
| rNLP_S45 | 224 | Yes | Not performed |
| rNLP_S46 | 225 | Yes | Not performed |
| rNLP_S47 | 228 | No | Not performed |
| rNLP_S48 | 229 | Yes | Not performed |
| rNLP_S49 | 230 | Yes | Not performed |
| rNLP_S50 | 231 | No | Not performed |
| rNLP_S51 | 232 | No | Not performed |
| rNLP_S52 | 233 | Yes | Not performed |
| rNLP_S53 | 237 | Yes | Not performed |
| rNLP_S54 | 238 | Yes | Not performed |
| rNLP_S55 | 239 | No | Not performed |
| rNLP_S56 | 240 | Yes | Not performed |
| rNLP_S57 | 241 | No | Not performed |
| rNLP_S58 | 242 | No | Not performed |
| rNLP_S59 | 243 | Yes | Not performed |
| rNLP_S60 | 244 | Yes | Not performed |
| rNLP_S61 | 245 | Yes | Yes |
| rNLP_S62 | 246 | Yes | Not performed |
| rNLP_S63 | 247 | Yes | Not performed |
| rNLP_S64 | 248 | No | Not performed |
| rNLP_S65 | 249 | Yes | Yes |
| rNLP_S66 | 250 | Yes | Not performed |
| rNLP_S67 | 251 | Yes | Yes |
| rNLP_S68 | 252 | Yes | Not performed |
| rNLP_S69 | 253 | Yes | Yes |
| rNLP_S70 | 254 | Yes | Not performed |
| rNLP_S71 | 259 | Yes | Not performed |
| rNLP_S72 | 270 | Yes | Not performed |
| rNLP_S73 | 272 | Yes | Not performed |
| rNLP_S74 | 271 | Yes | Not performed |

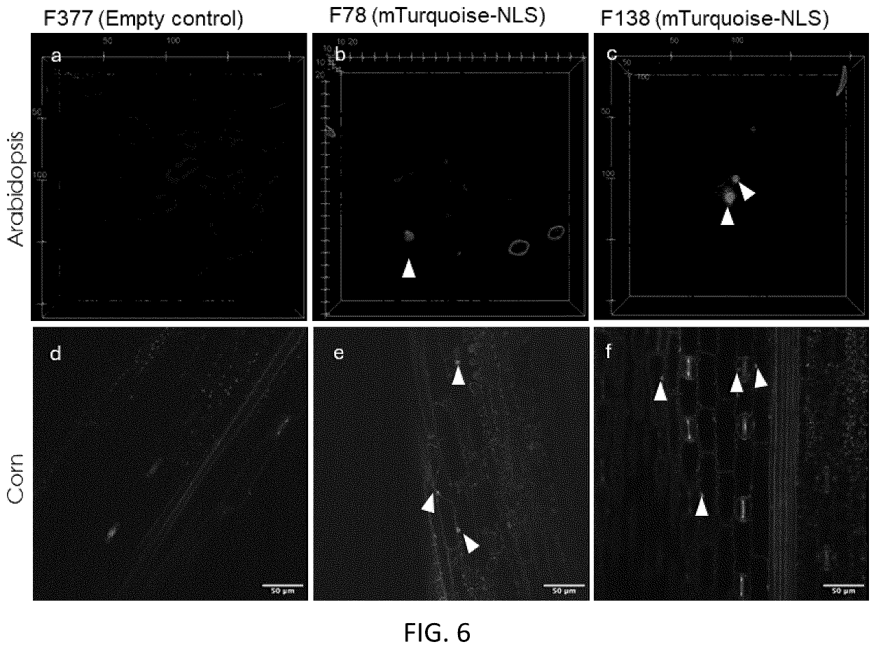

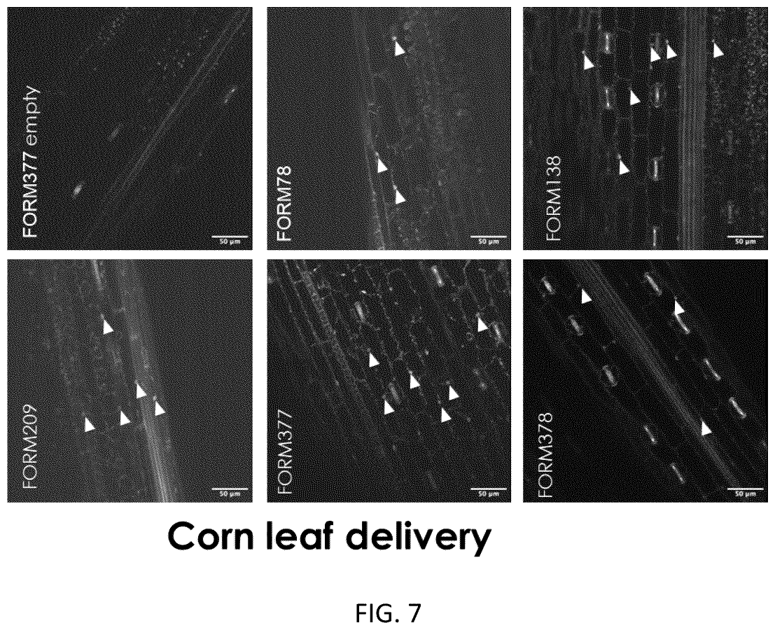

As shown in FIG. 6, expression of RNA encoding mTurquoise fused with the nuclear exportation signal (NLS) was detected in the nuclei of cells of corn and Arabidopsis leaves treated with FORM78 and FORM 138 indicating mRNA cargo was effectively delivered and translated. Further, as shown in FIG. 7, nuclear localization of RNA of the mTurquoise fused with the nuclear exportation signal (NLS) was observed in 3-week-old corn leaves treated with Form78, FORM 138, FORM29, FORM377, and Form378. Table 22 shows the compositions and physicochemical characteristics of NLP Formulations that effectively deliver RNA cargo to Arabidopsis and corn cells.

TABLE 22
Physicochemical characteristics and composition (molar %) of NLP compositions
that effectively deliver RNA cargo to Arabidopsis and corn leaf cells

| | | | Zeta | | | | | |
|---|---|---|---|---|---|---|---|---|
| FORM. | Size | | Potential | | Ionizable | Cationic | | |
| # | (nm) | PDI | (mV) | Cholesterol | Lipid | Lipid | PEG | Phospholipid |
| 167 | 158 | 0.237 | 11.3 | 38.5 | | 50 MC3 | 1.5 PEG2K | 10 DSPC |
| | | | | | | | PE 18:0 | |
| 199 | 133 | 0.269 | 11.4 | 20 | 60 DODAP | | 1.5 PEG2K | 18.5 DOPE |
| | | | | | | | PE 18:0 | |
| 201 | 230.7 | 0.405 | — | 20 | | 54.19 | 0.81 PEG5K | 25 MGDG |
| | | | | | | DODMA | PE 14:0 | |
| 209 | 180.3 | 0.188 | 5.37 | 40 | | 59.4 MC3 | 0.6 PEG2K | |
| | | | | | | | PE 18:0 | |
| 211 | 249.8 | 0.239 | 21.73 | 40 | | 40 DOTAP | | 20 DSPC |
| 245 | 1733.1 | 0.255 | 1.08 | 33.5 | 40 DODPA | | 1.5 PEG5K | 25 SF |
| | | | | | | | PE 14:0 | lecithin |
| 249 | 112.3 | 0.198 | 7.02 | 40 | 40 DODAP | | 1.5 PEG2K | 18.5 MGDG |
| | | | | | | | PE 18:0 | |
| 251 | 190.2 | 0.118 | 12.4 | 34.7 | | 40 MC3 | 0.3 PEG2K | 25 MGDG |
| | | | | | | | PE 18:0 | |
| 253 | 112.8 | 0.378 | 0.973 | 40 | | 49.25 MC3 | 1.5 PEG5K | 9.25 Hydro |
| | | | | | | | PE 14:0 | Soy PC |

### Example 30. Uptake and Systemic Distribution of Polynucleotides in Corn Seedlings

Movement of polynucleotide cargo encapsulated in selected NLP formulations to tissues distinct from the site of application was examined in corn seedlings.

### Experimental Procedure for Assaying Systemic Distribution of Polynucleotide Cargo in Corn Seedings:

### a) Seed Sterilization and Germination

Corn seeds were sterilized by immersion in ethanol 70% (2 min) followed by bleach (50%)+0.5% silwet in sterile water for 30 min. The seeds were then washed (5×) with sterile water and placed in autoclaved paper rolls deposited into a beaker containing half-strength MS liquid media, supplemented with vitamins (autoclaved) with 4% of PPM antifungal agent, to germinate at 28° C. in a chamber, covered with tinfoil.

b) dNLP Treatment of Corn Seedlings

4 days after sterilization, germinated seedlings were transferred from the paper rolls to 15 ml falcon tubes containing 1.5 ml of autoclaved half-strength MS media supplemented with vitamins and 2% of PPM antifungal and 0.5 ml of a treatment solution selected from: (a) a dNLP formulation as described in Table 23; (b) naked DNA; or (c) empty NLP (no cargo) or MS media (no NLP or cargo). Seedlings were transferred to the falcon tubes with care, so only the root contacted the treatment solution, and not the seed or aerial part. For this, given that 2 ml of volume in a 15 ml falcon is around 2-3 cm height, seedlings that show at least 5-6 cm of root length and at least 1-2 cm of mesocotyl+aerial part were treated. One seedling was treated per falcon tube, with as many falcon tubes as treatment solutions and replicates tested. The corn seedlings were treated at 28° in a chamber for 3 days, covered in tinfoil.

### c) Detection of Uptake and Distribution

After 3 days, root samples (taken from the submerged part of the root) and mesocotyl or shoot samples (from the aerial part of the seedling) were collected in individual tubes using sterile disposable blades, tweezers, and 2 ml Eppendorf tubes. The tissue was ground and DNA extraction was performed as follows:

1) Extraction buffer: (200 mM Tris-HCl, 250 mM NaCl, 25 mM EDTA, 0.5% SDS, in dH2O sterile) add 500 ul and vortex.

2) Add 130 ul of Potassium Acetate 3M, invert the tube 8×.

3) Centrifuge 15 min, 16000 g or more. The supernatant was transferred to a new labeled tube and the precipitate was discarded

4) 300 ul isopropanol was added to the supernatant, mixed by immersion, centrifuged 10 min at 16000 g. The supernatant was discarded and the pelleted precipitate was resuspended in 150 ul of 70% ethanol.

5) The resuspended pellet was centrifuged 10 min at 16000 g. The supernatant was discarded and full evaporation of the ethanol was achieved by leaving the tube opened overnight in a flow hood or by using a speedvac device (45 min, room temperature, alcoholic vacuum).

6) The pellet was resuspended in sterile water (50 ul) and PCR was performed using primers designed to detect the pBR322 cargo sequence.

7) The PCR results were run by standard gel electrophoresis. Detection of a band of the expected size (˜780 bp), depending on primers, indicated the presence of pBR322 DNA cargo in the sampled tissue.

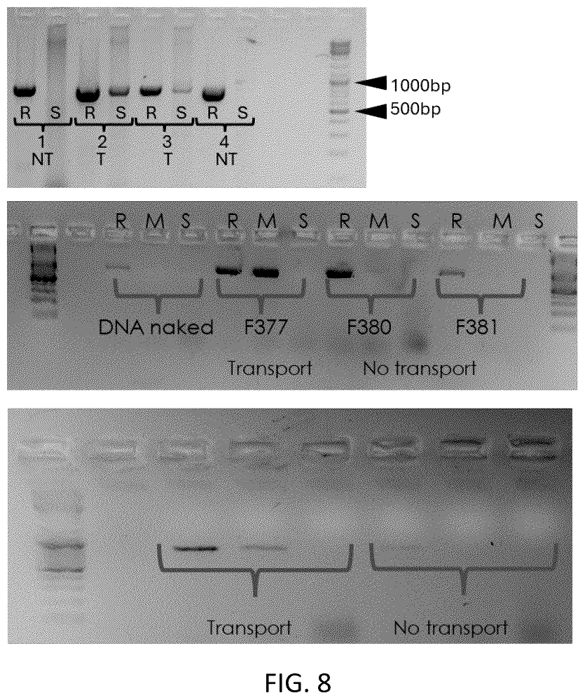

Results: As shown in FIG. 8, a band of the appropriate size indicating the presence of pBR322 DNA cargo in aerial tissues indicates transport of the DNA-NLP through the plant body after 3 days of incubation. The samples from the naked DNA control treatment showed a band of the appropriate size indicating the presence of pBR322 DNA cargo in root tissue but not in aerial tissue, indicating that pBR322 DNA was not distributed to tissues distal from the site of treatment. No band of band of the appropriate size was observed in any tissue from seedlings treated with empty NLP (no cargo) or MS media (no NLP or cargo) negative controls.

The results of assays to detect systemic delivery of polynucleotide cargo by NLPs as described herein is shown in Table 23.

TABLE 23
Detection of pBR322 DNA cargo in tissue
distal to the site of treatment

| | | Detection of | |
|---|---|---|---|
| | | Polynucleotide Cargo in | Number of |
| ID | Form | Distal Tissue | Replicates |
| rNLP_H1 | 27 | Not performed | Not performed |
| rNLP_H2 | 34 | Not performed | Not performed |
| rNLP_H3 | 29 | Not performed | Not performed |
| rNLP_H4 | 30 | Not performed | Not performed |
| rNLP_H5 | 32 | Not performed | Not performed |
| rNLP_H6 | 33 | Not performed | Not performed |
| rNLP_H7 | 31 | Not performed | Not performed |
| rNLP_H8 | 45 | Not performed | Not performed |
| rNLP_H9 | 46 | Not performed | Not performed |
| rNLP_H10 | 48 | Not performed | Not performed |
| rNLP_H11 | 49 | Not performed | Not performed |
| rNLP_H12 | 50 | Not performed | Not performed |
| rNLP_I1 | 47 | Not performed | Not performed |
| rNLP_I2 | 54 | Not performed | Not performed |
| rNLP_I3 | 55 | Not performed | Not performed |
| rNLP_I4 | 56 | Not performed | Not performed |
| rNLP_I5 | 57 | Not performed | Not performed |
| rNLP_I6 | 58 | Not performed | Not performed |
| rNLP_I7 | 59 | Not performed | Not performed |
| rNLP_I8 | 60 | Not performed | Not performed |
| rNLP_I9 | 61 | Not performed | Not performed |
| rNLP_I10 | 62 | Not performed | Not performed |
| rNLP_I11 | 63 | Not performed | Not performed |
| rNLP_I12 | 64 | Not performed | Not performed |
| rNLP_I13 | 65 | Yes | 1 |
| rNLP_I14 | 66 | Not performed | Not performed |
| rNLP_I15 | 74 | Not performed | Not performed |
| rNLP_I16 | 75 | Not performed | Not performed |
| rNLP_I17 | 76 | Not performed | Not performed |
| rNLP_I18 | 78 | Yes | 2 |
| rNLP_I19 | 79 | Yes | 1 |
| rNLP_I20 | 83 | Not performed | Not performed |
| rNLP_I21 | 84 | Not performed | Not performed |
| rNLP_I22 | 85 | Not performed | Not performed |
| rNLP_I23 | 86 | Not performed | Not performed |
| rNLP_I25 | 113 | Not performed | Not performed |
| rNLP_I26 | 130 | Not performed | Not performed |
| rNLP_I27 | 131 | Not performed | Not performed |
| rNLP_I28 | 132 | Not performed | Not performed |
| rNLP_I29 | 133 | Not performed | Not performed |
| rNLP_S1 | 114 | Not performed | Not performed |
| rNLP_S2 | 115 | Not performed | Not performed |
| rNLP_S3 | 116 | Not performed | Not performed |
| rNLP_S4 | 117 | Not performed | Not performed |
| rNLP_S5 | 118 | Not performed | Not performed |
| rNLP_S6 | 119 | Not performed | Not performed |
| rNLP_S7 | 120 | Not performed | Not performed |
| rNLP_S8 | 121 | Not performed | Not performed |
| rNLP_S9 | 122 | Not performed | Not performed |
| rNLP_S10 | 123 | Not performed | Not performed |
| rNLP_S11 | 124 | Not performed | Not performed |
| rNLP_S12 | 125 | Not performed | Not performed |
| rNLP_S13 | 126 | Not performed | Not performed |
| rNLP_S14 | 127 | Not performed | Not performed |
| rNLP_S15 | 128 | Not performed | Not performed |
| rNLP_S16 | 129 | Not performed | Not performed |
| rNLP_S17 | 134 | Yes | 1 |
| rNLP_S18 | 135 | Not performed | Not performed |
| rNLP_S19 | 136 | Not performed | Not performed |
| rNLP_S20 | 137 | Not performed | Not performed |
| rNLP_S21 | 138 | No | 2 |
| rNLP_S22 | 167 | Yes | 1 |
| rNLP_S23 | 189 | Not performed | Not performed |
| rNLP_S24 | 190 | Not performed | Not performed |
| rNLP_S25 | 191 | Not performed | Not performed |
| rNLP_S26 | 192 | Not performed | Not performed |
| rNLP_S27 | 193 | Not performed | Not performed |
| rNLP_S28 | 199 | No | 1 |
| rNLP_S29 | 200 | No | 1 |
| rNLP_S30 | 201 | Yes | 1 |
| rNLP_S31 | 202 | Not performed | Not performed |
| rNLP_S32 | 203 | No | 1 |
| rNLP_S33 | 209 | Yes | 1 |
| rNLP_S34 | 210 | No | 1 |
| rNLP_S35 | 211 | Yes | 1 |
| rNLP_S36 | 212 | No | 1 |
| rNLP_S37 | 216 | Not performed | Not performed |
| rNLP_S38 | 217 | Not performed | Not performed |
| rNLP_S39 | 218 | Not performed | Not performed |
| rNLP_S40 | 219 | Yes | 1 |
| rNLP_S41 | 220 | No | 1 |
| rNLP_S42 | 221 | Yes | 1 |
| rNLP_S43 | 222 | Yes | 1 |
| rNLP_S44 | 223 | Yes | 1 |
| rNLP_S45 | 224 | No | 1 |
| rNLP_S46 | 225 | No | 1 |
| rNLP_S47 | 228 | Not performed | Not performed |
| rNLP_S48 | 229 | No | 1 |
| rNLP_S49 | 230 | No | 1 |
| rNLP_S50 | 231 | Not performed | Not performed |
| rNLP_S51 | 232 | Not performed | Not performed |
| rNLP_S52 | 233 | Yes | 1 |
| rNLP_S53 | 237 | No | 1 |
| rNLP_S54 | 238 | Yes | 1 |
| rNLP_S55 | 239 | Not performed | Not performed |
| rNLP_S56 | 240 | No | 1 |
| rNLP_S57 | 241 | Not performed | Not performed |
| rNLP_S58 | 242 | Not performed | Not performed |
| rNLP_S59 | 243 | No | 1 |
| rNLP_S60 | 244 | No | 1 |
| rNLP_S61 | 245 | Yes | 1 |
| rNLP_S62 | 246 | Not performed | Not performed |
| rNLP_S63 | 247 | No | 1 |
| rNLP_S64 | 248 | Not performed | Not performed |
| rNLP_S65 | 249 | Not performed | Not performed |
| rNLP_S66 | 250 | Not performed | Not performed |
| rNLP_S67 | 251 | Not performed | Not performed |
| rNLP_S68 | 252 | Not performed | Not performed |
| rNLP_S69 | 253 | Not performed | Not performed |
| rNLP_S70 | 254 | Not performed | Not performed |
| rNLP_S71 | 259 | Not performed | Not performed |
| rNLP_S72 | 270 | Not performed | Not performed |
| rNLP_S73 | 272 | Not performed | Not performed |
| rNLP_S74 | 271 | Not performed | Not performed |
| rNLP_S75 | 273 | Not performed | Not performed |
| rNLP_S76 | 274 | Not performed | Not performed |
| rNLP_S77 | 275 | Not performed | Not performed |
| rNLP_S78 | 276 | Not performed | Not performed |
| rNLP_S79 | 277 | Not performed | Not performed |
| rNLP_S80 | 330 | Yes | 1 |
| rNLP_S81 | 331 | Not performed | Not performed |
| rNLP_S82 | 332 | No | 1 |
| dNLP_S54 | 365 | Yes | 1 |
| dNLP_S55 | 366 | No | 1 |
| dNLP_S56 | 367 | Not performed | Not performed |
| dNLP_S57 | 368 | Yes | 1 |
| dNLP_S58 | 369 | No | 1 |
| dNLP_S59 | 370 | Not performed | Not performed |
| dNLP_S60 | 371 | Not performed | Not performed |
| dNLP_S61 | 372 | Not performed | Not performed |
| dNLP_S62 | 373 | No | 1 |
| dNLP_S63 | 374 | Not performed | Not performed |
| dNLP_S64 | 375 | No | 1 |
| dNLP_S65 | 376 | No | 1 |
| dNLP_S66 | 377 | Yes | 4 |
| dNLP_S67 | 378 | Yes | 4 |
| dNLP_S68 | 379 | No | 1 |
| dNLP_S69 | 380 | No | 1 |
| dNLP_S70 | 381 | Yes | 1 |
| dNLP_S71 | 382 | No | 1 |
| dNLP_S72 | 383 | Yes | 1 |

TABLE 24
Physicochemical characteristics and composition (molar %) of NLP compositions
that promote systemic distribution of polynucleotide cargo.

| | | | Zeta | | | | | | |
|---|---|---|---|---|---|---|---|---|---|
| FORM. | Size | | Potential | | Ionizable | Cationic | | | |
| # | (nm) | PDI | (mV) | Cholesterol | Lipid | Lipid | PEG | Phospholipid | Cargo |
| 65 | 313.6 | 0.667 | 5.081 | 35.01 | | 21.49 | 0.6 | 25.04 | GFP RNA |
| | | | | | | DDAB | PEG5K | DOPE | |
| | | | | | | | PE 14:0 | | |
| 78 | 180.6 | 0.233 | 10.59 | 35.22 | | 21.62 | | 25.19 | GFP RNA |
| | | | | | | DDAB | | DOPE | |
| 79 | 96.26 | 0.213 | 20.73 | 42.63 | | 26.16 | 0.72 | 30.49 | GFP RNA |
| | | | | | | DDAB | PEG5K | DOPE | |
| | | | | | | | PE 14:0 | | |
| 134 | 284.3 | 0.135 | 3.72 | 34.47 | | 63.46 | 0.47 | 0.81 | SiRNA-GFP |
| | | | | | | DDAB | PEG5K | DOPE | |
| | | | | | | | PE 14:0 | 0.79 | |
| | | | | | | | | DSPC | |
| 167 | 158 | 0.237 | 11.3 | 38.5 | | 50 MC3 | 1.5 | 10 DSPC | Nluc RNA GFP |
| | | | | | | | PEG2K | | Nluc RNA Cyan |
| | | | | | | | PE 18:0 | | |
| 201 | 230.7 | 0.405 | — | 20 | | 54.19 | 0.81 | 25 | GFP RNA |
| | | | | | | DODMA | PEG5K | MGDG | Nluc RNA GFP |
| | | | | | | | PE 14:0 | | Nluc RNA Cyan |
| 209 | 180.3 | 0.188 | 5.37 | 40 | | 59.4 | 0.6 | | GFP RNA |
| | | | | | | MC3 | PEG2K | | Nluc RNA GFP |
| | | | | | | | PE 18:0 | | Nluc RNA Cyan |
| | | | | | | | | | SiRNA-GFP |
| 211 | 249.8 | 0.239 | 21.73 | 40 | | 40 | | 20 DSPC | GFP RNA |
| | | | | | | DOTAP | | | Nluc RNA GFP |
| | | | | | | | | | Nluc RNA Cyan |
| | | | | | | | | | SiRNA-GFP |
| 219 | 173.9 | 0.107 | 20.03 | 40 | 40 | | | 20 | GFP RNA |
| | | | | | DODAP | | | Hydro | Nluc RNA GFP |
| | | | | | | | | Soy PC | Nluc RNA Cyan |
| 221 | 214.5 | 0.51 | — | 40 | | 60 | | | GFP RNA |
| | | | | | | DODMA | | | Nluc RNA GFP |
| | | | | | | | | | Nluc RNA Cyan |
| 222 | 381.2 | 0.116 | — | 35 | | 40 | | 25 SF | GFP RNA |
| | | | | | | DDAB | | lecithin | Nluc RNA GFP |
| | | | | | | | | | Nluc RNA Cyan |
| 223 | 212.5 | 0.452 | — | 20 | | 53.5 | 1.5 | 25 SF | GFP RNA |
| | | | | | | DODMA | PEG5K | lecithin | Nluc RNA GFP |
| | | | | | | | PE 14:0 | | Nluc RNA Cyan |
| 233 | 118.4 | 0.358 | 3.85 | 20 | 60 | | 1.5 | 18.5 | GFP RNA |
| | | | | | DODAP | | PEG5K | DSPC | Nluc RNA GFP |
| | | | | | | | PE 14:0 | | Nluc RNA Cyan |
| 238 | 262.3 | 0.16 | 24.1 | 40 | | 56 | | 4 DOPE | GFP RNA |
| | | | | | | DDAB | | | Nluc RNA GFP |
| | | | | | | | | | Nluc RNA Cyan |
| 245 | 1733.1 | 0.255 | 1.08 | 33.5 | 40 | | 1.5 | 25 SF | GFP RNA |
| | | | | | DODPA | | PEG5K | lecithin | Nluc RNA GFP |
| | | | | | | | PE 14:0 | | Nluc RNA Cyan |
| 330 | — | — | — | 20 | | 53.5 | 1.5 | 25 SF | GFP RNA |
| | | | | | | DDAB | PEG2K | lecithin | Nluc RNA GFP |
| | | | | | | | PE 18:0 | | Nluc RNA Cyan |
| 365 | — | — | — | 40 | | 40 | | 20 | DNA |
| | | | | | | DDAB | | Hydro | |
| | | | | | | | | Soy PC | |
| 368 | — | — | — | 33.5 | | 40 MC3 | 1.5 | 25 DSPC | DNA |
| | | | | | | | PEG5K | | |
| | | | | | | | PE 14:0 | | |
| 377 | 4873 | 1 | −8.028 | 20 | | 60 | | 20 | GFP RNA |
| | | | | | | DOTAP | | MGDG | DNA |
| 378 | 136.7 | 0.252 | 0.223 | 20 | | 53.5 | 1.5 | 25 | GFP RNA |
| | | | | | | DDAB | PEG5K | DOPE | DNA |
| | | | | | | | PE 14:0 | | |
| 381 | — | — | — | 25.55 | | 60 | 1.5 | 12.95 | DNA |
| | | | | | | DODMA | PEG2K | MGDG | |
| | | | | | | | PE 18:0 | | |
| 383 | — | — | — | 40 | | 43.7 | 1.5 | 14.8 | DNA |
| | | | | | | DODMA | PEG5K | DOPE | |
| | | | | | | | PE 14:0 | | |

### Example 31. Predictive Modeling of NLP Performance

Random Forest modeling was used to identify important formulation parameters that predict the performance of NLP compositions in plant delivery applications. The models were trained on datasets comprising lipid composition, Synthetic Lipid to Nucleic Acid (N:P) ratio), and physical characteristics (e.g., particle size, polydispersity index (PDI) from previously tested NLP formulations.

For RNA-NLP (rNLP) uptake in Arabidopsis thaliana, the N:P ratio was identified as the single most influential variable, explaining approximately 17.3% of the variability in uptake. Not wishing to be bound by any particular theory, the ionizable and cationic synthetic lipids described herein may be incorporated in an NLP to enhance nucleic acid complexation, facilitate endosomal escape, and improve delivery into plant cells. The presence of certain cationic lipids, especially DDAB, correlated positively with uptake. Cholesterol, although frequently present, may have shown high importance due to its overrepresentation in the dataset.

For DNA-NLP (dNLP) systemicity in Zea mays (corn), particle size below 200 nm and low PDI values were identified as the most critical features associated with systemic movement. Among lipids, DODMA was the most predictive for systemicity, although it accounted for only about 6.5% of variability. Application of Random Forest modeling identified NLP formulations as described herein predicted to promote systemic distribution of polynucleotide cargo in plants. See Table 25.

TABLE 25
NLP Formulation Systemicity Predictions

| | Predicted | Prediction |
|---|---|---|
| FORM | Systemicity | Confidence |
| 74 | Yes | 0.93 |
| 75 | Yes | 0.92 |
| 49 | Yes | 0.88 |
| 50 | Yes | 0.87 |
| 76 | Yes | 0.87 |
| 83 | Yes | 0.87 |
| 115 | Yes | 0.85 |
| 86 | Yes | 0.83 |
| 125 | Yes | 0.83 |
| 118 | Yes | 0.82 |
| 124 | Yes | 0.82 |
| 61 | Yes | 0.81 |
| 63 | Yes | 0.81 |
| 121 | Yes | 0.81 |
| 27 | Yes | 0.8 |
| 66 | Yes | 0.8 |
| 218 | Yes | 0.8 |
| 62 | Yes | 0.78 |
| 29 | Yes | 0.77 |
| 84 | Yes | 0.77 |
| 60 | Yes | 0.76 |
| 54 | Yes | 0.74 |
| 252 | Yes | 0.74 |
| 34 | Yes | 0.73 |
| 32 | Yes | 0.73 |
| 85 | Yes | 0.72 |
| 131 | Yes | 0.72 |
| 113 | Yes | 0.7 |
| 116 | Yes | 0.7 |
| 132 | Yes | 0.69 |
| 133 | Yes | 0.69 |
| 228 | Yes | 0.68 |
| 30 | Yes | 0.67 |
| 114 | Yes | 0.67 |
| 117 | Yes | 0.67 |
| 129 | Yes | 0.67 |
| 123 | Yes | 0.66 |
| 56 | Yes | 0.65 |
| 57 | Yes | 0.65 |

### ENUMERATED EMBODIMENTS

1. A composition for delivering a heterologous functional agent to a plant cell, the composition comprising a plurality of NLPs, wherein each NLP comprises: at least one sterol; at least one quaternary ammonium salt lipid and/or at least one tertiary amine lipid; at least one phospholipid; at least one heterologous functional agent; wherein the NLP comprises a hydrophilic core, and wherein the heterologous functional agent is encapsulated in the NLP.

2. The composition of paragraph 1, wherein the sterol is cholesterol or sitosterol.

3. The composition of paragraph 1, wherein the at least one quaternary ammonium salt lipid is selected from the group consisting of SM-102 N-oxide, ALC-0315 N-oxide, Hexadecanedioic Acid Mono-L-carnitine Ester Chloride, Octadecanedioic Acid Mono-L-carnitine Ester Chloride, 14:0 TAP, 16:0 TAP, 18:0 TAP, DGTS (1,2-dipalmitoyl-sn-glycero-3-O-4′-[N,N,N-trimethyl]-homoserine), DC-6-14, 12:0 EPC, 14:0 EPC, 16:0 EPC, 18:0 EPC, 16:0-18:1 EPC, 1-Palmitoyl-2-arachidoyllecithin, iPhos-lipid1, 18:0 DDAB (DDAB), DOTAP, DOTMA, DODAC, DORI, DOBAQ, MVL5 and DOSPA.

4. The composition of paragraph 1, wherein the at least one quaternary ammonium salt lipid is DOTAP or DDAB.

5. The composition of paragraph 1, wherein the at least one tertiary amine lipid is selected from the group consisting of 113-O12B, 113-O16B, 14:0 DAP, 16:0 DAP, 1O14, 246C10, 304O13, 306-N16B, 306-O12B, 306-O12B-3, 306Oi10, 306Oi9-cis2, 4A3-SC8503O13, 80-O16B, 93-O17O, 93-O17S, 98N12-5, 9A1P9, 98N12-5, A12-Iso5-2DC18, A18-Iso5-2DC18, AA3-DLin, AA-T3A-C12, Al-28, ALC-0315 (ALC-315), ATX-001, ATX-100, BAMEA-O16B, C12-113, C12-200, C12-SPM, C13-112-tetra-tail, C13-112-tri-tail, C13-113-tetra-tail, C13-113-tri-tail, C14-4, C14-SPM, C3-K2-E14, cKK-E12, cKK-E12, cKK-E15, CL1, DLin-DMA, DLin-KC2-DMA (KC2), D-Lin-MC2-DMA, D-Lin-MC3-DMA (MC3), D-Lin-MC4-DMA, DODAP (18:1), DODMA, DOG-IM4, FITS, G0-C14, GL67, IAJD249, IAJD93, IC8, iPhos-lipid2, iPhos-lipid3, iPhos-lipid4, L13, L14, L15, L16, L2, L3, L319, L9, Lipid 10, Lipid 14, Lipid 16, Lipid 2, Lipid 23, Lipid 29, Lipid 5, Lipid 8, Lipid A4, Lipid A6, Lipid A9, Lipid AX4, Lipid C24, Lipid Catechol, Lipid III-45, Lipid R6, LP01, MVL5, NT1-014B, OC2-K3-E10, OF-02, OF-C4-Deg-Lin, OF-Deg-Lin, PPZ-A10, RCB-4-8, RM 133-3, RM 137-15, SM-102, SSPalmM, SSPalmO-Phe, TCL053, TT3, YK-009, YSK05, and ZA3-Ep10.

6. The composition of paragraph 5, wherein the at least one tertiary amine lipid is selected from the group consisting of MC3, KC2, DODMA, SM-102 and ALC-315.

7. The composition of paragraph 1, wherein the at least one phospholipid is DOPE, DSPC, soybean lecithin, MGDG, Hydro Soy PC, or sunflower lecithin.

8. The composition of paragraph 7, wherein the soybean lecithin is de-oiled soybean lecithin.

9. The composition of paragraph 7, wherein the sunflower lecithin is de-oiled sunflower lecithin.

10. The composition of paragraph 1, wherein the composition further comprises a surface modifier.

11. The composition of paragraph 10, wherein the surface modifier is a pegylated lipid.

12. The composition of paragraph 11, wherein the pegylated lipid is selected from the group consisting of PEG5K PE 14:0, PEG2K PE 14:0, and PEG2K PE 18:0.

13. The composition of paragraph 11, wherein the pegylated lipid is present at a molar percentage of about 0.1-10%.

14. The composition of paragraph 1, wherein the composition further comprises a pectin derivative.

15. The composition of paragraph 14, wherein the pectin derivative is a hydrophobically modified pectin derivative.

16. The composition of paragraph 14, wherein the pectin derivative is present at a molar percentage of about 0.1-10%.

17. The composition of paragraph 1, wherein the composition further comprises a boron acid lipid.

18. The composition of paragraph 17, wherein the boron acid lipid is present at a molar percentage of about 0.1-10%.

19. The composition of paragraph 1, wherein the composition comprises 25-45 mol % of at least one sterol, 15-60 mol % of at least one synthetic lipid, and 5-55 mol % of at least one phospholipid.

20. The composition of paragraph 19, wherein the composition comprises about 35 mol % of at least one sterol, about 25 mol % of at least one synthetic lipid, and about 35 mol % of at least one phospholipid.

21. The composition of any of paragraphs 1-20, wherein the heterologous functional agent is a polynucleotide.

22. The composition of paragraph 21, wherein the polynucleotide is chosen from an mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), an ADAR targeting oligonucleotide, an antisense oligonucleotide, a long non-coding RNA, a ceDNA, a minicircle, a miniplasmid, a viroid, a virus, or a DNA molecule encoding any of these RNAs.

23. The composition of paragraph 21, wherein the polynucleotide is an mRNA, a siRNA, or a gRNA.

24. The composition of paragraph 21, wherein the polynucleotide is a plasmid.

25. The composition of any of paragraphs 1-24, wherein the size of the NLPs ranges between 50 and 500 nm.

26. The agricultural composition of any of paragraphs 1-24, wherein the NLPs comprise at least one a phospholipid bilayer.

27. The agricultural composition of any of paragraphs 1-24, wherein the NLPs have a micellar structure.

28. The composition of any of paragraphs 1-24, wherein the zeta potential of the NLPs ranges between 0 and +50 mV.

29. The composition of any of paragraphs 1-24, wherein the zeta potential of the NLPs ranges between 0 and −50 mV.

30. The composition of any of paragraphs 1-24, wherein the composition further comprises an unencapsulated heterologous functional agent.

31. The composition of paragraph 30, wherein the unencapsulated heterologous functional agent is selected from the group consisting of a pesticidal agent, a fertilizing agent, a herbicidal agent, a plant-modifying agent, an insect attractant, a plant growth promoting agent, a biostimulant, and a plant immunity elicitor.

32. The composition of paragraph 1, wherein the NLP comprises a synthetic lipid to nucleic acid (N:P) ratio optimized for uptake in a plant cell.

33. The composition of paragraph 32, wherein the synthetic lipid is a synthetic amphiphilic lipid or a functionalized lipid.

34. The composition of paragraph 32, wherein the synthetic lipid is a cationic lipid or an ionizable lipid.

35. The composition of paragraph 1, wherein the NLP comprises DDAB to promote cellular uptake.

36. The composition of paragraph 1, wherein the NLP comprises DODMA to promote systemic movement in a plant.

37. The composition of paragraph 1, wherein the NLP has a mean particle size of less than 200 nm.

38. The composition of paragraph 1, wherein the NLP has a PDI of less than 0.3.

39. A NLP composition for delivering a polynucleotide to a plant cell, the composition comprising a molar percentage of about 35 mol % cholesterol, about 22 mol % DDAB, about 25 mol % DOPE, and about 18 mol % sunflower lecithin, and a polynucleotide.

40. The composition of paragraph 39, wherein the polynucleotide is an mRNA, an siRNA or a gRNA.

41. A composition for delivering at least one heterologous functional agent to a plant cell, the composition comprising a first and a second plurality of NLPs, wherein each plurality of NLPs has the composition of any of paragraphs 1-40, and wherein the first and the second plurality of NLPS each comprise different heterologous functional agents.

42. A composition for use in delivering a heterologous functional agent to a plant cell, the composition comprising a plurality of NLPs, wherein each NLP comprises: at least one sterol; at least one synthetic lipid; at least one phospholipid; and a heterologous functional agent. wherein the NLP comprises a hydrophilic core, and wherein the heterologous functional agent is encapsulated in the NLP.

43. A composition comprising a plurality of NLPs, wherein the NLPs are produced by a process comprising the steps of: (a) providing an organic phase comprising: at least one sterol; at least one synthetic lipid; at least one phospholipid; (b) providing an aqueous phase comprising at least one heterologous functional agent; and (c) mixing the organic phase and the aqueous phase; thereby forming the NLPs.

44. A method for delivering a heterologous functional agent to a plant cell, the method comprising: contacting the plant cell with the NLP composition of any one of paragraphs 1-42; thereby delivering the heterologous functional agent to the plant cell.

45. A method for increasing the uptake of a heterologous functional agent by a plant cell, the method comprising: (a) encapsulating a heterologous functional agent in an NLP comprising: at least one sterol; at least one synthetic lipid; at least one phospholipid; and (b) contacting the plant cell with the NLP composition comprising the heterologous functional agent; thereby increasing the uptake of the heterologous functional agent as compared to a heterologous agent not comprised in an NLP composition.

46. A method for increasing the fitness of a plant, the method comprising: delivering to the plant an effective amount of the composition of any one of paragraphs 1-42, wherein the method increases the fitness of the plant relative to an untreated plant.

47. A method for decreasing the fitness of a plant, the method comprising: delivering to the plant an effective amount of the composition of any one of paragraphs 1-42, wherein the method decreases the fitness of the plant relative to an untreated plant.

48. A method for modifying a nucleic acid in a plant cell, the method comprising: contacting the plant cell with the composition of any one of paragraphs 21-29.

49. The method of paragraph 48, wherein the plant comprises an expression cassette for a CRISPR endonuclease.

50. A method of delivering a heterologous functional agent to the meristem of a plant, the method comprising: contacting the plant with a composition comprising a plurality of lipid nanoparticles (NLPs), wherein each NLP comprises: (a) at least one sterol; (b) at least one cationic lipid and/or at least one ionizable lipid; (c) at least one phospholipid; and (d) at least one heterologous functional agent; wherein the NLPs are formulated for delivery to the meristem, and wherein the method results in the delivery of the heterologous functional agent to one or more meristematic cells of the plant.

51. The method of paragraph 50, wherein the composition is applied directly to a shoot apical meristem (SAM), a root apical meristem (RAM), or both.

52. The method of paragraph 50, wherein the heterologous functional agent is selected from a polynucleotide, a polypeptide, a small molecule, or a plant growth regulator.

53. The method of paragraph 52, wherein the polynucleotide is selected from an mRNA, siRNA, shRNA, antisense oligonucleotide, or a CRISPR guide RNA.

54. The method of paragraph 52, wherein the polypeptide is a transcription factor, a cell cycle regulator, or a fluorescent reporter protein.

55. The method of paragraph 52, wherein the small molecule is selected from salicylic acid, gibberellic acid, or an auxin.

56. The method of paragraph 50, wherein the NLPs have a mean particle size of less than 200 nanometers.

57. The method of paragraph 50, wherein the composition is applied by foliar spray, injection, dipping, or seed coating.

58. The method of paragraph 50, wherein the delivery of the functional agent alters gene expression in the meristem.

59. A method of delivering a heterologous functional agent systemically in a plant, the method comprising: contacting the plant with a composition comprising a plurality of lipid nanoparticles (NLPs), wherein each NLP comprises: (a) at least one sterol; (b) at least one cationic lipid and/or at least one ionizable lipid; (c) at least one phospholipid; and (d) at least one heterologous functional agent; wherein the NLPs are formulated for delivery to one or more plant tissues away from the site of contact, and wherein the method results in the delivery of the heterologous functional agent to one or more tissues of the plant.

60. The method of paragraph 59, wherein the NLP comprises the cationic lipid DODMA.

61. The method of paragraph 59, wherein the NLP comprises a tertiary amine lipid selected from the group consisting of DODMA, MC3, and KC2.

62. The method of paragraph 59, wherein the NLP comprises a polydispersity index (PDI) of less than 0.2.

63. The method of paragraph 59, wherein the NLPs have a mean particle diameter between 80 nm and 150 nm.

64. The method of paragraph 59, wherein the NLP comprises a pegylated lipid selected from PEG2K PE 14:0, PEG2K PE 18:0, or PEG5K PE 14:0 at a molar ratio of 0.1-5%.

65. The method of paragraph 59, wherein the NLP comprises a phospholipid selected from DOPE, DSPC, or sunflower lecithin.

66. The method of paragraph 59, wherein the NLP comprises a sterol selected from cholesterol or sitosterol at a molar percentage of 25% to 45%.

67. The method of paragraph 59, wherein the NLP comprises a combination of DODMA, cholesterol, DOPE, and sunflower lecithin in molar ratios selected to promote systemic delivery.

68. The method of paragraph 59, wherein the NLP further comprises a surface modifier selected from a PEGylated lipid or a hydrophobically modified pectin.

69. The method of paragraph 59, wherein the NLP comprises less than 5 mol % surface modifier and exhibits enhanced movement into vascular tissues.

1: A composition for delivering a heterologous functional agent to a plant cell, the composition comprising a plurality of NLPs, wherein each NLP comprises:
at least one sterol;
at least one quaternary ammonium salt lipid and/or at least one tertiary amine lipid;
at least one phospholipid;
at least one heterologous functional agent;
wherein the NLP encapsulates a hydrophilic core comprising the heterologous functional agent.

2: The composition of claim 1, wherein the sterol is cholesterol or sitosterol.

3: The composition of claim 1, wherein the at least one quaternary ammonium salt lipid is selected from the group consisting of SM-102 N-oxide, ALC-0315 N-oxide, Hexadecanedioic Acid Mono-L-carnitine Ester Chloride, Octadecanedioic Acid Mono-L-carnitine Ester Chloride, 14:0 TAP, 16:0 TAP, 18:0 TAP, DGTS (1,2-dipalmitoyl-sn-glycero-3-O-4′-[N,N,N-trimethyl]-homoserine), DC-6-14, 12:0 EPC, 14:0 EPC, 16:0 EPC, 18:0 EPC, 16:0-18:1 EPC, 1-Palmitoyl-2-arachidoyllecithin, iPhos-lipid1, 18:0 DDAB (DDAB), DOTAP, DOTMA, DODAC, DORI, DOBAQ, MVL5 and DOSPA.

4: The composition of claim 1, wherein the at least one quaternary ammonium salt lipid is DOTAP or DDAB.

5: The composition of claim 1, wherein the at least one tertiary amine lipid is selected from the group consisting of 113-O12B, 113-O16B, 14:0 DAP, 16:0 DAP, 1O14, 246C10, 304O13, 306-N16B, 306-O12B, 306-O12B-3, 306Oi10, 306Oi9-cis2, 4A3-SC8503O13, 80-O16B, 93-O017O, 93-O17S, 98N12-5, 9A1P9, A12-Iso5-2DC18, A18-Iso5-2DC18, AA3-DLin, AA-T3A-C12, Al-28, ALC-0315 (ALC-315), ATX-001, ATX-100, BAMEA-O16B, C12-113, C12-200, C12-SPM, C13-112-tetra-tail, C13-112-tri-tail, C13-113-tetra-tail, C13-113-tri-tail, C14-4, C14-SPM, C3-K2-E14, cKK-E12, cKK-E15, CL1, DLin-DMA, DLin-KC2-DMA (KC2), D-Lin-MC2-DMA, D-Lin-MC3-DMA (MC3), D-Lin-MC4-DMA, DODAP (18:1), DODMA, DOG-IM4, FITS, G0-C14, GL67, IAJD249, IAJD93, IC8, iPhos-lipid2, iPhos-lipid3, iPhos-lipid4, L13, L14, L15, L16, L2, L3, L319, L9, Lipid 10, Lipid 14, Lipid 16, Lipid 2, Lipid 23, Lipid 29, Lipid 5, Lipid 8, Lipid A4, Lipid A6, Lipid A9, Lipid AX4, Lipid C24, Lipid Catechol, Lipid III-45, Lipid R6, LP01, MVL5, NT1-O14B, OC2-K3-E10, OF-02, OF-C4-Deg-Lin, OF-Deg-Lin, PPZ-A10, RCB-4-8, RM 133-3, RM 137-15, SM-102, SSPalmM, SSPalmO-Phe, TCL053, T3, YK-009, YSK05, and ZA3-Ep10.

6: The composition of claim 5, wherein the at least one tertiary amine lipid is selected from the group consisting of MC3, KC2, DODMA, SM-102 and ALC-315.

7: The composition of claim 1, wherein the at least one phospholipid is DOPE, DSPC, soybean lecithin, MGDG, Hydro Soy PC, or sunflower lecithin.

8: The composition of claim 7, wherein the soybean lecithin is de-oiled soybean lecithin, or wherein the sunflower lecithin is de-oiled sunflower lecithin.

9. (canceled)

10: The composition of claim 1, wherein the composition further comprises a surface modifier.

11: The composition of claim 10, wherein the surface modifier is a pegylated lipid selected from the group consisting of PEG5K PE 14:0, PEG2K PE 14:0, and PEG2K PE 18:0.

12-13. (canceled)

14: The composition of claim 1, wherein the composition further comprises a pectin derivative, and/or wherein the composition further comprises a boronic acid lipid.

15-18. (canceled)

19: The composition of claim 1, wherein the composition comprises 25-45 mol % of at least one sterol, 15-60 mol % of at least one quaternary ammonium salt lipid and/or at least one tertiary amine lipid, and 5-55 mol % of at least one phospholipid.

20. (canceled)

21: The composition of claim 1, wherein the heterologous functional agent is a polynucleotide.

22: The composition of claim 21, wherein the polynucleotide is chosen from an mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), an ADAR targeting oligonucleotide, an antisense oligonucleotide, a long non-coding RNA, a ceDNA, a minicircle, a miniplasmid, a viroid, a virus, or a DNA molecule encoding any of these RNAs.

23-34. (canceled)

25: The composition of claim 1, wherein the size of the NLPs ranges between 50 and 500 nm.

26: The composition of claim 1, wherein the NLPs comprise at least one phospholipid bilayer, and/or wherein the NLPs have a micellar structure.

27. (canceled)

28: The composition of claim 1, wherein the zeta potential of the NLPs ranges between 0 and +50 mV, or wherein the zeta potential of the NLPs ranges between 0 and −50 mV.

29. (canceled)

30: The composition of claim 1, wherein the composition further comprises an unencapsulated heterologous functional agent.

31: The composition of claim 30, wherein the unencapsulated heterologous functional agent is selected from the group consisting of a pesticidal agent, a fertilizing agent, a herbicidal agent, a plant-modifying agent, an insect attractant, a plant growth promoting agent, a biostimulant, and a plant immunity elicitor.

32-36. (canceled)

37: A method for delivering a heterologous functional agent to a plant cell, the method comprising:
contacting the plant cell with the NLP composition of claim 1;
thereby delivering the heterologous functional agent to the plant cell.

38-42. (canceled)


SEQUENCE LISTING (2 sequences)

SEQ ID NO: 1 - RNA, 22 nt, synthetic construct
  source 1..22: mol_type = other RNA
ggcatcaaagccaacttcaaaa

SEQ ID NO: 2 - RNA, 22 nt, synthetic construct
  source 1..22: mol_type = other RNA
ttgaagttggctttgatgccgt